(19) United States
(12) Patent Application Publication
(10) Pub. No.: US 2026/0232367 A1
(43) Pub. Date: Aug. 13, 2026

(54) Methods and Devices for Endovascular Treatment

(71) Applicant: Virender K. Sharma, Paradise Valley, AZ (US)

(72) Inventor: Virender K. Sharma, Paradise Valley, AZ (US)

(21) Appl. No.: 19/440,487

(22) Filed: Jan. 5, 2026

Related U.S. Application Data
(60) Provisional application No. 63/741,921, filed on Jan. 5, 2025.
(60) Provisional application No. 63/769,461, filed on Mar. 10, 2025.

(51) Int. Cl. A61B 18/04; A61B 17/00; A61B 17/22; A61B 18/00; A61M 25/10

(52) U.S. Cl. CPC A61B 18/04; A61B 2017/00292; A61B 17/22012; A61B 2017/22062; A61B 2018/00255; A61B 2018/00386; A61B 2018/00577; A61B 2018/00696; A61B 2018/00994; A61B 2018/046; A61M 2025/1013

(57) ABSTRACT

Methods and devices for treating primary stenosis, restenosis, and in-stent restenosis in a vessel include a catheter with two or more balloons that applies a dilation force in combination with a thermal ablative energy and, preferably, acoustic pressure, to a diseased vessel segment, resulting in mechanical dilation and thermal ablation of majority of the diseased intima, media, neointima. The balloon is made of a material that can sustain both high dilation pressure and ablation temperatures without affecting its integrity or functionality. A controller controls the delivery of fluid and thermal energy to the balloon. An inner balloon is inflated with a heated liquid solution to deliver the thermal energy, and an outer balloon is inflated and used to apply acoustic pressure to target tissue.

### CROSS-REFERENCE

The present application relies on U.S. Patent Provisional Application No. 63/769,461, titled “Hybrid Thermal Balloon Angioplasty and Intravascular Lithotripsy Catheter and Method of Use” and filed on Mar. 10, 2025, and U.S. Patent Provisional Application No. 63/741,921, titled “Methods and Devices for Endovascular Treatment”, and filed on Jan. 5, 2025, for priority. The above-mentioned applications are herein incorporated by reference in their entirety.

### FIELD

The present specification is related generally to the field of treating endovascular disease. More specifically, the present specification is related to devices for endovascular treatment of vascular stenosis due to atherosclerotic vascular disease and in-stent restenosis and to devices that integrate thermal angioplasty with the application of localized pressure.

### BACKGROUND

Atherosclerotic vascular disease is a condition in which plaque accumulates inside a person's arteries. This plaque is made of fat, cholesterol, calcium, and other substances found in the blood. Over time, the plaque hardens and narrows the arteries, reducing blood flow to vital organs and tissues. The disease can affect arteries throughout the body, leading to serious health issues such as coronary artery disease (heart attack), stroke, and peripheral artery disease. Risk factors include high cholesterol, hypertension, smoking, diabetes, and a sedentary lifestyle.

In-stent restenosis (ISR) occurs when an artery that was previously treated with a stent becomes narrowed again due to the growth of neoendothelium or scar tissue within the stent. This neo-endothelium or scar tissue accumulates inside the stent, restricting blood flow. ISR typically develops months to years after stent implantation, and it can cause symptoms similar to the original blockage, such as chest pain or shortness of breath. Treatment options include angioplasty, placement of a new stent (bare metal or drug eluting), or drug-coated balloon therapy to prevent further narrowing. Risk factors include diabetes, stent type, and artery size.

Atherosclerotic vascular disease is typically treated through a combination of lifestyle changes, medications, and, in more severe cases, surgical interventions. Patients are encouraged to adopt a heart-healthy lifestyle that includes a balanced diet, regular exercise, and smoking cessation to reduce further plaque buildup. Medications such as statins are commonly prescribed to lower cholesterol levels, while antiplatelet drugs, like aspirin, help prevent blood clots. Blood pressure medications may also be necessary to control hypertension, and diabetes management is crucial for those with high blood sugar. In cases where the disease significantly restricts blood flow, procedures like angioplasty, where a stent is inserted to widen the artery, or bypass surgery, which reroutes blood flow around the blockage, may be required.

In-stent restenosis, the re-narrowing of an artery after a stent has been placed, is usually treated by additional procedures to reopen the artery. One option is balloon angioplasty, where a balloon is inflated inside the narrowed stent to restore blood flow. Another approach is the placement of a drug-eluting stent, which slowly releases medication to prevent scar tissue from forming. Drug-coated balloons, which also release anti-proliferative drugs during the procedure, can be used as an alternative to a stent. In some cases, atherectomy, a procedure to remove neo-endothelium or recurrence or progression of atherosclerotic disease and/or scar tissue, is performed. Medications, such as antiplatelet therapy and statins, continue to play a role in preventing further complications.

Despite advances in the treatment of atherosclerotic vascular disease and in-stent restenosis, high failure and recurrence rates remain a significant concern. Many patients experience restenosis, where arteries re-narrow after treatments like stenting or balloon angioplasty. This recurrence is driven by factors such as the body's response to the stent, neo-endothelium or scar tissue formation, and underlying conditions such as diabetes. Current treatment modalities often fail to prevent long-term complications. As a result, patients may require repeated procedures, leading to increased morbidity, higher mortality rates, and substantial healthcare costs. The chronic nature of the disease and the frequent need for re-intervention place a substantial burden on both patients and healthcare systems, highlighting the need for improved long-term solutions.

FIG. 1A illustrates representations of an artery structure 102, a vein structure 104, and an artery anatomy 106. A typical artery structure 102, represented in an elongated cylindrical form, includes an outer layer 108 that coaxially encompasses external elastic membrane 110 which coaxially surrounds and protects smooth muscle 112. Inside muscle 112 is a basement membrane 114 which contains the subendothelial layer 116. The hollow lumen formed within the artery is coaxially encompassed by subendothelial layer 116.

Structure 104 of a vein comprises an endothelium layer 118, coaxially surrounded by basement membrane 120, which is further coaxially encompassed in a combination 122 of smooth muscles and elastic fibers. An outer coat 124 coaxially forms the outermost layer of structure 104.

Anatomy 106 of an artery can also be classified in three distinct coaxial layers—an outermost layer called Tunica adventitia 126, a middle layer called Tunica media 128, and an innermost layer called Tunica intimate 130.

Atherosclerosis involves the build-up of plaque inside the wall of the artery (or arteries). The formation of plaque results in hardening of the arteries. The pathogenesis of endothelial plaque formation (atherosclerosis) is a complex process involving multiple steps and cellular interactions. Endothelial dysfunction plays a crucial role in the initiation and progression of atherosclerotic plaque formation. FIG. 1B illustrates stages 132 of atherosclerosis and its progression 134 due to endothelial dysfunction. Stages 132 show the developmental stages of atherosclerosis through a cross-sectional view of an artery. A healthy stage 136 has an absence of any type of plaque formation inside the artery. The process of plaque formation begins with damage to the endothelium, often caused by factors like smoking, diabetes, or high blood pressure. This damage results in decreased nitric oxide (NO) production, which impairs the endothelium's normal protective functions. A stage 138 shows where formation of a fatty streak shows deposits of lipids 140 and formation of a foam cell 142. Low-density lipoprotein (LDL) cholesterol enters the dysfunctional endothelium. As a result, the dysfunctional endothelium becomes more permeable, allowing LDL cholesterol to enter the subendothelial space. The process of LDL transcytosis across the endothelial cell layer is mediated by caveolin-1 (Cav-1), which enables vesicle formation. In the next stage 144, fibrofatty plaque is seen forming inside the artery. In the subendothelial space, LDL is oxidized by macrophages and smooth muscle cells. Oxidized LDL acts as a danger-associated molecular pattern (DAMP), triggering immune responses. Activated endothelial cells upregulate adhesion molecules such as E-selectin, ICAM, and VCAM. These molecules facilitate leukocyte rolling and adhesion to the endothelial surface. The presence of oxidized LDL triggers the release of growth factors, cytokines, and upregulation of adhesion molecules. These factors attract monocytes to the site of injury. Monocytes differentiate into macrophages, which engulf the oxidized LDL. This process transforms macrophages into lipid-laden foam cells. Foam cells and other immune cells release pro-inflammatory cytokines, perpetuating the inflammatory response. Foam cell 142 evolves into a smooth-muscle cell 146 with a core 148. Smooth muscle cells from the media layer migrate to the intima and proliferate. These cells produce extracellular matrix components like collagen and elastin. As foam cells accumulate and undergo apoptosis, they form a lipid-rich necrotic core. Further, smooth muscle cells and extracellular matrix form a fibrous cap over the lipid core.

The plaque continues to grow through ongoing lipid accumulation, inflammation, and cellular proliferation. Continued inflammation and matrix degradation can lead to thinning of the fibrous cap, making the plaque vulnerable to rupture. Therefore, the disease may progress to a stage 150 where complicated plaques are known to form. As the plaque thickens, it becomes hypoxic, stimulating the growth of new blood vessels (angiogenesis) from the vasa vasorum. In advanced stages, calcium deposits 152 may form within the plaque. Continued inflammation and matrix degradation can lead to thinning of the fibrous cap, making the plaque vulnerable to rupture and resulting in thrombus 154.

Progression 134 of atherosclerosis shows the formation of foam cells 156 and fatty streak 158 that typically occurs within the first decade of onset of atherosclerosis. Lipid accumulation fosters the growth to a preatheroma stage 160 and further to an atheroma 162 within the third decade. When the progression is still not checked, atherosclerosis advances to formation of fibro-atheroma 164 and then complicated lesions 166 within the fourth decade from the onset. Smooth muscle cells from the media layer migrate to the intima and proliferate. These cells produce extracellular matrix components like collagen and elastin at this stage 164. As foam cells accumulate and undergo apoptosis, they form a lipid-rich necrotic core. Smooth muscle cells and extracellular matrix form a fibrous cap over the lipid core. The plaque continues to grow through ongoing lipid accumulation, inflammation, and cellular proliferation. Continued inflammation and matrix degradation can lead to thinning of the fibrous cap, making the plaque vulnerable to rupture. Dysfunctional endothelium expresses pro-coagulatory tissue factor (TF) and anti-fibrinolytic plasminogen activator inhibitor (PAI-1), which creates a thrombogenic vascular environment. Progression to development of complicated plaques at stage 166 illustrates these developments. Rupture of the thin fibrous cap or erosion of the endothelium covering the plaque can expose thrombogenic material to the bloodstream. Exposure of the plaque contents to blood can trigger rapid thrombus formation, potentially leading to acute events like myocardial infarction or stroke.

The pathogenesis, illustrated through FIGS. 1A and 1B, highlights the central role of endothelial dysfunction that initiates a cascade of events that promote atherosclerotic plaque formation, lipid accumulation, inflammation, and cellular interactions in the formation and progression of the atherosclerotic plaques. Understanding these mechanisms is crucial for developing targeted therapies to prevent and treat atherosclerosis.

A simplified three-tier grading system for primary stenosis can be derived from the more detailed classifications. A first classification is termed as mild stenosis, in which 0-49% narrowing of the vessel lumen can occur. Mild stenosis is typically asymptomatic or minimally symptomatic and often corresponds to grades I and II in more detailed systems of classification. A second classification is termed ‘moderate stenosis’, in which 50-69% narrowing of the vessel lumen can occur. Moderate stenosis may cause intermittent symptoms and corresponds to grades III and IV in some known classification systems. A third classification is termed ‘severe stenosis’, in which 70-100% narrowing of the vessel lumen can occur. Severe stenosis is often symptomatic and may require intervention and includes grades V through VIII in more detailed classifications. The simplified classification system described herein, aligns with clinical decision-making thresholds, as stenosis above 50% is often considered significant, and stenosis above 70% is generally regarded as severe and more likely to require intervention.

There are several surgical and interventional procedures used to treat atherosclerotic blockages in arteries. One method is angioplasty and stenting, also known as percutaneous coronary intervention (PCI). Angioplasty and stenting is a minimally invasive procedure that involves inserting a catheter with a small balloon into the blocked artery. FIG. 2 illustrates a series of representative drawings showing the angioplasty and stenting procedure. Referring to the figure, coronary artery 202 is seen located on the surface of heart 204. At step 206, a cross-sectional view along a longitudinal axis of the artery 202 is shown where presence of plaque 208 is detected and a catheter 210 is inserted to position a closed stent 212 proximate the plaque 208. Another cross-sectional view 214 taken along an axis perpendicular to the longitudinal axis, shows a narrowed space within artery 202 resulting from presence of plaque 208. Stent 212 is inserted within the narrowed channel in artery 202 in a closed configuration, to enable positioning of the stent proximate to the plaque 208. At step 216, a balloon 218 positioned inside the stent 212 is inflated, which results in expansion of stent 212. The illustration of step 216 shows stent 212 in its expanded configuration. The balloon 218 is inflated to compress the plaque 208 against the wall of artery 202, thereby widening the vessel and enabling an increased blood flow. Stent 212 is often a small mesh tube with the function to keep the artery open, when it is in its expanded configuration. Another cross-sectional view 222 along an axis perpendicular to the longitudinal axis of artery 202, shows a widened artery 202 achieved by the positioning of stent 212 within the vessel. The process of angioplasty and stenting can provide rapid relief of symptoms like chest pain. This method of treatment is often used as an emergency treatment during heart attacks. Recovery time from angioplasty and stenting is shorter compared to that from an open surgery. The procedure is associated with high technical success rates (97-100% for stenoses, 87% for total occlusions) with 1-year primary patency rates around 88-89% and low rates of major adverse events. Further, angioplasty and stenting have better outcomes compared to balloon angioplasty alone. Bare metal stents, drug eluting metal and biodegradable polymer coated stents, and bioresorbable drug eluting stents may be used for this procedure.

Another method for surgical and interventional procedures used to treat atherosclerotic blockages in arteries is known as endarterectomy. This procedure is specifically used for carotid artery. Endarterectomy is an open surgical procedure to remove plaque buildup from inside an artery. During the procedure, a surgeon makes an incision to access the affected artery, opens the artery and removes the inner lining containing the plaque, and finally closes the artery to restore normal blood flow. This procedure is commonly used to treat severe carotid artery disease to prevent strokes. The procedure is known to reduce 5-year risk of ipsilateral stroke in asymptomatic patients with >60% stenosis. Perioperative risk of stroke or death should be <3% for optimal benefit.

Yet another form of surgical intervention is known as Coronary Artery Bypass Graft (CABG). CABG is an open-heart surgery procedure that creates a new route for blood to flow around blocked coronary arteries. In a typical procedure, a blood vessel is taken from another part of the body (often the leg or chest). The taken vessel is attached above and below the blocked portion in the artery (‘blockage’) to bypass it. Multiple bypasses may be done depending on the number of blocked arteries. The CABG procedure provides better long-term outcomes for patients with multiple severe blockages compared to stenting. Also, this procedure improves survival in patients with left main coronary artery disease or triple-vessel disease. Furthermore, use of arterial grafts (especially left internal thoracic artery) improves long-term outcomes. 10-year graft patency rates are known to be >90% for arterial grafts and 50-60% for venous grafts. However, this procedure may result in complications including 1-2% risk of a stroke, 1% risk of a wound infection, graft failure, renal dysfunction (2-3%) risk, and atrial fibrillation (20-50% risk), and is highly invasive.

Still another known surgical interventional procedure is atherectomy. Atherectomy is a catheter-based procedure that physically removes plaque from the artery. There are different types of atherectomy procedures. One of the types of atherectomy is rotational atherectomy, which uses a diamond-tipped burr to pulverize hard plaque. Another type is directional atherectomy, which cuts and removes plaque pieces. Atherectomy procedures are generally used less frequently than angioplasty/stenting. These procedures are known to provide high technical success rates (100% in one study). Atherectomy procedure often require additional treatments like balloon angioplasty or stenting. These procedures can reduce rates of flow-limiting dissection and bailout stenting compared to angioplasty alone. Atherectomy is known to have 30-day patency rates of approximately 83%.

Atherosclerotic vascular disease presents significant treatment challenges, particularly with calcified plaques resistant to mechanical dilation. Still another known interventional procedure to treat calcified stenosis is intravascular lithotripsy (IVL), wherein pulsatile shock waves or acoustic pressure waves are used to create minor fissures or cracks in a calcified plaque or calcium deposits in the arteries which is subsequently dilated with balloon angioplasty or followed with a stent placement.

The procedure involves using a balloon angioplasty catheter with lithotripsy emitters which are inserted into an artery. The balloon is inflated to a low pressure level, though which lithotripsy emitters deliver sonic pressure waves in pulses. The balloon is deflated between the pulses. Unfortunately, extended inflation durations in IVL procedures have been associated with increased ischemic burden, particularly in coronary applications. One of the outcomes of the extended inflation times include hemodynamic strain. 30-second IVL cycles lead to significantly increased myocardial oxygen demand, with myocardial pH dropping to 6.8 within 15 seconds. Extended cycle durations correlate with 28% higher ischemic burden in peripheral arteries and 50% ATP depletion in subendocardial layers during coronary interventions. Another possible outcome is the arrhythmogenic risk. In this risk, ventricular capture incidence increases from 12% in 10-second cycles to 41% in 30-second cycles. Furthermore, prolonged occlusion results in higher incidences of ventricular tachycardia (VT) and atrial fibrillation. Yet another possible outcome of extended inflation durations in IVL procedures is the occurrence of ischemic events. During ischemic events, ST-segment elevations are more pronounced with longer cycles (2.1 mm vs. 0.8 mm in shorter cycles). Also, myocardial ischemia risk escalates significantly with cycle durations exceeding 15 seconds. In practice, intentional heating during an IVL procedure is avoided. IVL systems are designed to not apply any form of heating to avoid thermal damage. Known IVL catheters employ temperature regulation mechanisms to maintain balloon temperature stability and explicitly teaches against any physiologically meaningful heating of the vessel wall during lithotripsy procedures.

### In-Stent Restenosis

Another issue that may surface after a treatment involving a stent is known as in-stent restenosis (ISR), which involves significant reduction in the luminal diameter of a previously stented coronary artery segment. Angiographically, ISR is typically defined as >50% diameter stenosis within the stented segment or within 5 millimeters (mm) of the stent edges. Using intravascular imaging, ISR is defined as ≥75% narrowing of the reference vessel area in cross-section. The epidemiology of ISR has evolved over time with advances in stent technology. In the bare-metal stent (BMS) era, ISR occurred in approximately 20-35% of cases. With first-generation drug-eluting stents (DES), ISR rates decreased to 5-10%. Even with modern second-generation DES, ISR rates remain around 5-10%. ISR currently accounts for about 5-10% of all percutaneous coronary intervention (PCI) procedures performed in clinical practice. The time course of ISR development differs between stent types. BMS-ISR typically peaked within the first 6 months after implantation. Whereas, DES-ISR tends to occur later and can continue to develop for several years after implantation.

Certain factors are associated with higher ISR risk. Patient factors include diabetes, chronic kidney disease, and heart failure. Lesion factors include smaller vessel size, long lesions, and complex lesions. Additionally, procedural factors, such as, stent under-expansion, stent fracture, and multiple stents, may increase ISR risk. In summary, while modern stent technology has reduced ISR rates compared to the BMS era, ISR remains a persistent clinical challenge.

There are several biological factors that often interact and contribute to varying degrees in the development of ISR, depending on patient characteristics, stent type, and other clinical factors. One of the main biological factors that contribute to ISR is inflammation. ISR is primarily driven by a non-specific inflammatory response to vessel wall injury caused by stent implantation. This chronic inflammation stimulates various processes leading to neointimal hyperplasia. Neointimal hyperplasia involves the proliferation and migration of vascular smooth muscle cells (VSMCs) from the media to the intima, along with extracellular matrix production. It is a key mechanism in ISR development. Another factor contributing to ISR is neoatherosclerosis, which refers to the formation of new atherosclerotic lesions within the neointima of stented segments. Neoatherosclerosis can occur in both bare-metal stents (BMS) and drug-eluting stents (DES), though it tends to develop more rapidly in DES. Further, another factor is endothelial dysfunction in which damage to the endothelium during stenting can impair its normal functions, contributing to the restenotic process.

Platelet activation and thrombosis is also among the main factors contributing to ISR. The initial thrombotic response after stenting is associated with later development of restenosis. Various growth factors (such as, PDGF, FGF, TGF-β) and inflammatory cytokines stimulate VSMC proliferation and migration, thereby contributing to ISR. Moreover, excessive extracellular matrix (ECM) synthesis contributes significantly to neointimal growth. Also, certain genetic polymorphisms may increase susceptibility to ISR. Delayed or incomplete healing of the endothelium can also promote ongoing inflammation and neointimal growth, thereby contributing to ISR. Additionally, increased oxidative stress in the vessel wall can contribute to the restenosis process.

It is also known that smooth muscle cells (SMCs) play a central and critical role in the development of ISR. Involvement of SMCs includes phenotypic switching, wherein upon vascular injury from stent implantation, SMCs switch from a quiescent “contractile” phenotype to a proliferative “synthetic” phenotype. This phenotypic modulation is a key initiating event in ISR.

Additionally, activated SMCs migrate from the media layer to the intima, contributing to neointimal formation. This migration is stimulated by various growth factors and cytokines released after injury. Further, SMCs in the synthetic state undergo excessive proliferation within the neointima. This proliferation is a major contributor to the narrowing of the vessel lumen. Synthetic SMCs produce excessive extracellular matrix components, further contributing to neointimal growth and vessel narrowing. Moreover, SMCs participate in the inflammatory response by producing cytokines and growth factors that perpetuate the restenotic process. In later stages of ISR, especially with drug-eluting stents, SMCs can contribute to the formation of neoatherosclerotic lesions within the neointima. Also, while drug-eluting stents inhibit SMC proliferation initially, delayed healing can lead to a prolonged inflammatory response and eventual SMC activation.

The central role of SMCs in ISR is highlighted by the fact that many anti-restenotic therapies, including those used in drug-eluting stents, specifically target SMC proliferation and migration. Understanding the complex biology of SMCs in the context of vascular injury and repair remains crucial for developing more effective strategies to prevent and treat ISR.

Stent restenosis primarily involves the growth of neointimal tissue within and around the stent. The main tissue responsible for ISR is neointimal tissue, which consists of Vascular smooth muscle cells (VSMCs) and Extracellular matrix (ECM) rich in proteoglycans. The neointimal layer begins forming approximately two weeks after stent implantation, growing over and between the stent struts. Bare-metal stents tend to have higher rates of neointimal proliferation compared to drug-eluting stents, which release anti-proliferative drugs to inhibit this process.

The neointimal tissue in restenosis has several notable features regarding composition and characteristics. One feature is that it is primarily composed of VSMCs and ECM. Another feature is that scar tissue may form underneath the healthy neointimal lining. Also, the neointimal tissue can become excessively thick, obstructing blood flow and causing clinical symptoms of restenosis.

Restenotic lesions have been identified with different tissue patterns using OCT imaging techniques. Homogenous tissue patterns are associated with more stable tissue and heterogenous tissue patterns are associated with increased fibrin deposits and loose connective tissue.

In some cases, especially in late restenosis, neointimal tissue can develop features of neoatherosclerosis. In this condition, lipid pools may form within the neointima. Ruptured neoatherosclerosis with intracoronary thrombus can occur. The neointimal tissue growth typically occurs within 3-6 months after stent placement, though it can continue to develop over time. Understanding the nature of this tissue growth is crucial for developing effective treatments and prevention strategies for ISR. Therefore, neointimal proliferation plays a central role in ISR development. Understanding this process enables development of targeted strategies to prevent or treat restenosis.

After stent implantation, a new layer of tissue called neointima begins to form over and between the stent struts. This neointimal tissue consists primarily of vascular smooth muscle cells (VSMCs) and extracellular matrix (ECM). Vascular injury from stenting triggers VSMCs to proliferate and migrate from the media layer into the developing neointima. This process starts approximately two weeks after stenting. The proliferating VSMCs produce large amounts of ECM proteins, contributing to the growth of the neointimal layer. As the neointimal tissue continues to grow and thicken over time, it can progressively narrow the vessel lumen, leading to restenosis.

The inflammatory reaction to stent implantation also plays an important role and stimulates neointimal proliferation. Inflammatory cells release growth factors and cytokines that promote VSMC proliferation and ECM production.

Dynamic remodeling and altered composition of the ECM following vascular injury create an environment that promotes SMC phenotypic modulation, proliferation, migration, and survival, ultimately contributing to their accumulation in the intima. The ECM plays several important roles in contributing to SMC accumulation in the intima during the development of in-stent restenosis. Vascular injury triggers degradation of the existing ECM by matrix metalloproteinases (MMPs) and other proteases. The ECM breakdown allows SMCs to migrate from the media to the intima. After an injury, a provisional matrix composed of fibrin, fibronectin, and other plasma proteins forms. The matrix provides a scaffold for SMC adhesion and migration into the intima. The composition of the ECM changes in the developing neointima, with increased deposition of proteins like fibronectin. These ECM changes promote a synthetic SMC phenotype associated with increased proliferation and migration. SMCs interact with the ECM via integrins. Different ECM proteins activate specific integrin signaling pathways that can modulate SMC phenotype, proliferation, and migration. Also, the ECM can bind and sequester growth factors like PDGF, which can then be released to stimulate SMC proliferation and migration. Further, changes in ECM stiffness and composition can alter mechanotransduction signaling in SMCs, promoting a synthetic phenotype. Synthetic SMCs produce more ECM components, further promoting the synthetic phenotype, creating a positive feedback loop. The newly synthesized ECM provides a substrate for SMC migration into the developing neointima. Moreover, interactions between SMCs and the ECM via integrins can promote cell survival and resistance to apoptosis.

Similar to primary stenosis, a classification system for ISR can also be derived. As discussed above, in a first classification, mild ISR is associated with 0-49% narrowing of the vessel lumen. Mild ISR is usually asymptomatic or minimally symptomatic and often corresponds to grades I and II in more detailed systems. In a second classification, moderate ISR is associated with 50-69% narrowing of the vessel lumen. Moderate ISR may cause intermittent symptoms and corresponds to grades III and IV in detailed classification systems. In a third classification, severe ISR results in 70-100% narrowing of the vessel lumen. Severe ISR is often symptomatic and may require intervention. Further, severe ISR corresponds to grades V through VIII in more detailed classification systems. The simplified classification system described herein aligns with clinical decision-making thresholds, since stenosis above 50% is often considered significant, and stenosis above 70% is generally regarded as severe and more likely to require intervention.

Ablation techniques for treating neointimal tissue in cases of ISR have been tried with minimal success due to interference caused by the metal stent with the ablation process. Additionally, most of the known ablation techniques have limited the ablation or resection predominantly to the neointimal tissue inside the stent lumen and has not address the diseased tissue outside the stent lumen specially on the outside of the struts of the stent in the vessel wall. None of the known techniques effectively address the defects in the media of a blood vessel.

The repair process after an ablation aims to restore the protective endothelial layer and vessel integrity. However, excessive neointimal formation can lead to restenosis in some cases. Balance between healing and excessive proliferation determines the long-term outcome of the ablation site. Key factors that influence the repair process include extent of initial injury, local hemodynamic forces, systemic factors (such as, inflammation, diabetes) and individual genetic predisposition.

The process of healing of the vascular intima after an ablation procedure is achieved in steps. The first and immediate response includes endothelial denudation at the ablation site followed by platelet adhesion and aggregation that forms a temporary protective layer. Also, inflammatory cells are recruited to the injury site. The next step is the acute phase, which may last from a few hours to a few days. In the acute phase, inflammatory response intensifies with increased cytokine and growth factor release after which the SMCs in the media begin to dedifferentiate from a contractile to a synthetic phenotype. The acute phase is followed by the subacute phase that may last from a few days to a few weeks. During the subacute phase, SMCs proliferate and migrate from the media to the intima. Subsequently the ECM production increases, particularly proteoglycans and hyaluronan and endothelial cells begin to proliferate and migrate to cover the denuded area. Following the subacute phase is the chronic phase, which may last from weeks to months. In the chronic phase, SMC proliferation and ECM deposition in the intima can continue. Gradual re-endothelialization of the injured area is also expected. Further, remodeling of the newly formed neointima can occur. In the next step, SMCs return to a more quiescent state and ECM composition shifts towards a more mature, collagen-rich structure. After that the endothelial layer is fully restored, though it may remain dysfunctional for some time.

Endothelial dysfunction from the diseased vessel wall plays a critical role in contributing to intimal hyperplasia through several key mechanisms. First, endothelial dysfunction results in reduced production of nitric oxide (NO) and prostacyclin, which normally inhibit SMC proliferation and migration. Impaired production of other vasoprotective mediators, such as hydrogen sulfide (H₂S), further disrupts vascular homeostasis. Second, dysfunctional endothelial cells secrete pro-inflammatory cytokines and chemokines that recruit and activate inflammatory cells. The inflammatory environment promotes SMC dedifferentiation and proliferation. Third, injured endothelial cells release growth factors like PDGF, bFGF, and TGF-β that stimulate SMC proliferation and migration. Also, endothelial dysfunction alters the composition of the ECM, promoting a more synthetic SMC phenotype. Further, loss of the protective endothelial layer leads to platelet adhesion and activation, thereby releasing additional growth factors. Moreover, dysfunctional endothelium has reduced capacity to regenerate and cover the injured area, prolonging the inflammatory and proliferative responses. The altered vascular environment promotes SMC switching from a contractile to a synthetic phenotype, increasing their proliferation and migration into the intima. Additionally, increased reactive oxygen species production contributes to SMC proliferation and migration. Endothelial injury triggers mobilization of progenitor cells that can contribute to neointima formation.

In summary, endothelial dysfunction in the diseased vessel wall creates a pro-inflammatory, pro-proliferative environment that stimulates SMC migration and proliferation, leading to intimal hyperplasia. These mechanisms create a pro-inflammatory, pro-proliferative environment that stimulates SMC migration, proliferation and extracellular matrix production, leading to intimal hyperplasia. Restoring normal endothelial function is thus a key target for preventing intimal hyperplasia.

Several key differences are known in neointima formation between normal and atherosclerotic endothelium. Normal endothelium maintains vascular homeostasis through production of nitric oxide (NO) and other factors that inhibit inflammation, cell proliferation, and thrombosis. Atherosclerotic endothelium is dysfunctional, with reduced NO production and increased inflammation, leading to a pro-proliferative environment. In normal vessels, the inflammatory response after injury is typically acute and resolves. Atherosclerotic vessels have chronic inflammation, with increased expression of adhesion molecules and pro-inflammatory cytokines, promoting sustained neointima formation. SMCs in normal vessels are predominantly in a “contractile” phenotype. In atherosclerotic vessels, SMCs are more likely to be in a “synthetic” phenotype, prone to proliferation and migration. Normal vessels have a structured ECM that helps maintain SMC quiescence. Atherosclerotic vessels have altered ECM composition, promoting SMC migration and proliferation. Both normal and atherosclerotic vessels can recruit progenitor cells, but this process may be enhanced in atherosclerotic conditions. In atherosclerotic vessels, there is a higher risk of neo atherosclerosis within the neointima, especially with drug-eluting stents. Neointima formation in normal vessels typically peaks earlier and may be resolved more completely. In atherosclerotic vessels, the process can be more prolonged and less likely to fully resolve.

In summary, while the basic processes of neointima formation are similar, atherosclerotic endothelium provides a more conducive environment for sustained and exaggerated neointimal growth due to pre-existing inflammation, endothelial and SMC dysfunction, and altered cellular phenotypes.

The therapeutic goal of treatments for neointimal hyperplasia and ISR is to inhibit smooth muscle cell (SMC) proliferation and migration, reduce inflammation, and prevent excessive neointimal hyperplasia while promoting proper vessel healing. In clinical practice, the choice of treatment depends on various factors including lesion characteristics, previous treatments, patient comorbidities, and local expertise. A combination of approaches is often used, particularly for complex or recurrent cases. Ongoing research aims to further improve outcomes and develop novel targeted therapies for neointimal hyperplasia and ISR.

However, current treatment options do not adequately or effectively address the changes in the intima, neointima, or the media and leave the diseased intima, neointima, or the media, which has abnormal regeneration and proliferation potential, unaddressed in a patient. Additionally, debulking therapies can only remove the intima which is in the lumen of the stent while the diseased intimal and media, and SMC, beneath the stent from which the neointima generates cannot be effectively or adequately removed. Resulting neointima harbor the same baseline defects and makes it prone to atherosclerosis and restenosis.

Therefore, it is desirable to be able to eliminate the diseased SMC, endothelium and/or media in substantial amount so that the neointima originate from the healthy stem cells residing in the non-diseased intima or media or adventitia or in the bone marrow (depending on extent and depth of ablation) and has a low risk of intimal hyperplasia and restenosis.

It is also desirable to be able to maintain the SMCs predominantly in a “contractile” phenotype or convert SMCs into a “synthetic” phenotype, prone to proliferation and migration, into a “contractile” phenotype.

It is desirable to be able to maintain the structured ECM that helps maintain SMC quiescence and inhibit SMC migration and proliferation.

It is desirable to activate apoptotic factors and the TGF-β/Smad-2 inflammatory pathway with thermal balloon angioplasty, which will affect vessel remodeling.

It is also desirable to increases the expression of Bax and Caspase-3 while decreasing Bcl-2 levels, promoting apoptosis, which will help maintain arterial vasodilation.

It is also desirable to expedite the normal healing process and regeneration of normal healthy neointima after a vascular intervention to reduce the duration of required antiplatelet therapy and reduce a patient's risk of bleeding.

It is further desirable to eliminate substantial portion of the diseased tunica intima and/or tunica media without significantly ablating the tunica adventitia of the vessel wall, hence preventing complications such as vessel wall perforation or vessel wall stenosis from scar tissue formation or vessel wall aneurysm.

In certain situations, it may be desirable to ablate the tunica adventitia of a patient's diseased blood vessel, however, placement of scaffolding such as a metal or bioabsorbable or biodegradable stent post-ablation will be needed to prevent complications such as stricture formation, aneurysmal dilation, or perforation of the vessel wall.

It is desirable to be able to eliminate the diseased intima or media in the presence of a vascular stent or scaffolding and/or not preventing a subsequent, immediate or delayed vascular stent placement.

It is also desirable to protect the blood in the vessel lumen from thermal injury from the ablative process, hence preventing and or eliminating char formation and or subsequent thromboembolic events.

It is desirable to be able to perform the balloon angioplasty and thermal ablation of the disease intima in atherosclerotic vascular stenosis and ISR using a single device, eliminating the need for multiple device exchanges and device repositioning, hence decreasing the risk of infection, air-embolization during multiple catheter changes, and thromboembolic events with manipulation of multiple devices across a stenotic lesion.

It is desirable to deploy methods and devices that minimize IVL cycle times while maintaining therapeutic efficiency.

### SUMMARY

The following embodiments and aspects thereof are described and illustrated in conjunction with systems, tools and methods, which are meant to be exemplary and illustrative, and not limiting in scope. The present application discloses numerous embodiments.

The present specification discloses a system for treating in-stent restenosis in a lumen of a patient, comprising: a catheter having a distal end, wherein the distal end comprises an inner inflatable member defining an inner chamber configured to receive a working liquid and an outer inflatable member enclosing the inner inflatable member and defining an outer chamber configured to contain an acoustic coupling fluid; and a controller coupled to the catheter and configured to activate and control an acoustic wave source coupled to the distal end and configured to deliver lithotripsy pulses that propagate through the acoustic coupling fluid and through the outer inflatable member, activate and control a thermal energy source configured to heat the working liquid in the inner chamber; and one or more pumps for delivering the working liquid and the acoustic coupling fluid to the distal end.

Optionally, the controller is further configured to control at least one of: (i) a parameter of the thermal energy source, (ii) a parameter of the acoustic wave source, or (iii) a pressure or a volume of the outer chamber. Optionally, the controller is further configured to adjust the pressure or the volume of the outer chamber to control at least one of an amount of ablative energy passing from the inner chamber to the outer chamber or an acoustic coupling condition for the lithotripsy pulses comprising an acoustic path length or attenuation through the acoustic coupling fluid.

Optionally, the inner inflatable member is inflatable to a dilation pressure sufficient to dilate the lumen, and wherein the outer inflatable member is inflatable to a supra-dilation pressure greater than the dilation pressure.

Optionally, the acoustic wave source comprises at least one emitter disposed within the outer chamber or coupled to the distal end to transmit pulses into the acoustic coupling fluid.

Optionally, the controller is configured to perform a therapy sequence comprising causing the outer inflatable member to be inflated and the inner inflatable member to be inflated and causing the working liquid to be heated and delivered to the distal end during a first period, wherein, in the course of the first period, the working liquid's temperature is held substantially constant. Optionally, the controller is configured to perform a therapy sequence comprising causing the acoustic wave source to deliver lithotripsy pulses that propagate through the acoustic coupling fluid during a second period. Optionally, the second period initiates at a beginning of the first period and ends at an end of the first period. Optionally, the second period initiates at a beginning of the first period and after 30% to 50% of the first period elapses. Optionally, the second period initiates after 50% of the first period elapses and ends at an end of the first period.

The present specification also discloses a method for treating in-stent restenosis in a lumen of a patient, comprising: providing a catheter having a distal end, wherein the distal end comprises an inner inflatable member defining an inner chamber configured to receive a working liquid and an outer inflatable member enclosing the inner inflatable member and defining an outer chamber configured to contain an acoustic coupling fluid; using a controller, causing the acoustic coupling fluid to be delivered to the outer chamber; using the controller, causing the working liquid to be delivered and heated in the inner chamber during a first period, wherein a temperature of the working liquid is substantially constant during the first period; and using the controller, causing the acoustic wave source to deliver lithotripsy pulses that propagate through the acoustic coupling fluid during a second period.

Optionally, the second period initiates at a beginning of the first period and ends at an end of the first period.

Optionally, the second period initiates at a beginning of the first period and after 30% to 50% of the first period elapses.

Optionally, the second period initiates after 50% of the first period elapses and ends at an end of the first period.

Optionally, 50-100% of the patient's intima in the lumen is removed and more than 10% of the patient's intima, media, smooth muscle cells, and/or other tissue located beneath a stent is removed. Optionally, more than 50% of the patient's tunica intima in the lumen and more than 25% of the patient's tunica media in the lumen is ablated without ablating more than 25% of the patient's tunica adventitia in the lumen.

Optionally, the method further comprises controlling at least one of: (i) a parameter of the thermal energy source, (ii) a parameter of the acoustic wave source, or (iii) a pressure or a volume of the outer chamber. Optionally, the method further comprises adjusting the pressure or the volume of the outer chamber to control at least one of an amount of ablative energy passing from the inner chamber to the outer chamber or an acoustic coupling condition for the lithotripsy pulses comprising an acoustic path length or attenuation through the acoustic coupling fluid.

Optionally, the method further comprises inflating the inner inflatable member to a dilation pressure sufficient to dilate the lumen, and inflating the outer inflatable member to a supra-dilation pressure greater than the dilation pressure.

Optionally, the acoustic wave source comprises at least one emitter disposed within the outer chamber or coupled to the distal end to transmit pulses into the acoustic coupling fluid.

The present specification also discloses a treatment system comprising: a catheter comprising: an elongate shaft; an outer balloon coupled to the elongate shaft; an inner balloon positioned within the outer balloon; an inner balloon fluid path in fluid communication with the inner balloon; and an outer balloon fluid path in fluid communication with the outer balloon; and an external system comprising: an energy delivery circuit configured to deliver electrical energy to heat a working fluid associated with the inner balloon; a fluid delivery subsystem configured to deliver fluid through the inner balloon fluid path and the outer balloon fluid path; at least one temperature sensor interface configured to receive a temperature signal associated with the catheter; at least one pressure sensor interface configured to receive at least one pressure signal associated with at least one of the inner balloon or the outer balloon; and a controller configured to execute instructions that, when executed, cause the controller to: control an inflation state of the outer balloon via the fluid delivery subsystem; control an inflation state of the inner balloon via the fluid delivery subsystem; and control the energy delivery circuit based at least in part on the temperature signal to regulate a temperature associated with the working fluid.

Optionally, the controller regulates the temperature to a selected setpoint within a range of 50° C. to 80° C. Optionally, the selected setpoint is 65° C.

Optionally, the controller regulates the temperature within +2° C. of the selected setpoint.

Optionally, the controller controls at least one of an inner balloon pressure or an outer balloon pressure within a range of 2 atm to 10 atm.

Optionally, the controller is configured to support a maximum pressure up to 100 atm.

Optionally, the fluid delivery subsystem comprises at least one pump comprising at least one of a syringe pump, a piston pump, a peristaltic pump, a diaphragm pump, and a gear pump.

Optionally, the fluid delivery subsystem comprises a first pump configured to control the inner balloon and a second pump configured to control the outer balloon.

Optionally, the at least one temperature sensor interface is configured to receive the temperature signal from a sensor positioned within the inner balloon, within the outer balloon, or within the elongate shaft.

Optionally, the controller is configured to execute a therapy cycle comprising a ramp interval, a hold interval, and a deflation interval. Optionally, the ramp interval is 5 seconds, the hold interval is 20 seconds, and the deflation interval is 5 seconds. Optionally, the therapy cycle has a total duration of 30 seconds.

Optionally, the controller causes deflation of the inner balloon to complete before deflation of the outer balloon.

Optionally, deflation of the inner balloon completes 1 second before deflation of the outer balloon.

Optionally, the catheter comprises one or more intravascular lithotripsy emitters associated with the outer balloon and the external system is configured to deliver electrical drive signals to energize the one or more intravascular lithotripsy emitters. Optionally, the controller is configured to deliver a pulse train having a pulse repetition frequency of 1 Hz. Optionally, the controller is configured to deliver the pulse train during at least one portion of a hold interval of the therapy cycle. Optionally, the controller is configured to deliver the pulse train during an early portion of the hold interval. Optionally, the controller is configured to deliver the pulse train during an entire duration of the hold interval. Optionally, the controller is configured to deliver the pulse train during a later portion of the hold interval.

Optionally, the catheter comprises one or more pulsed field ablation electrodes incorporated at a distal end of the catheter and the external system comprises a pulsed energy delivery stage configured to deliver pulsed electric field energy to the one or more pulsed field ablation electrodes. Optionally, the pulsed energy delivery stage is configured to deliver a programmable pulse train including at least pulse count and pulse width. Optionally, the pulsed energy delivery stage is configured to deliver bipolar pulses or biphasic pulses.

The present specification also discloses an external system configured to couple to a catheter comprising an inner balloon and an outer balloon, the external system comprising: an energy delivery circuit; a fluid delivery subsystem; at least one temperature sensor interface; at least one pressure sensor interface; and a controller configured to execute instructions that, when executed, cause the controller to: control an inflation state of at least one of the inner balloon or the outer balloon via the fluid delivery subsystem; and control the energy delivery circuit based at least in part on a temperature signal received by the at least one temperature sensor interface to regulate a temperature associated with a working fluid of the inner balloon.

Optionally, the controller is configured to regulate the temperature to a selected setpoint within a range of 50° C. to 80° C. and within ±2° C. of the selected setpoint.

The present specification also discloses a method of treating a vessel with a catheter comprising an inner balloon positioned within an outer balloon, the method comprising: inflating at least one of the outer balloon or the inner balloon; heating a working fluid associated with the inner balloon using an external system; regulating a temperature associated with the working fluid to a selected setpoint within a range of 50° C. to 80° C.; and maintaining the temperature within ±2° C. of the selected setpoint for a treatment interval.

Optionally, the selected setpoint is 65° C. Optionally, the method further comprises delivering intravascular lithotripsy pulses at 1 Hz during at least a portion of the treatment interval. Optionally, the intravascular lithotripsy pulses are delivered during an early portion of a hold interval, during an entire duration of the hold interval, or during a later portion of the hold interval. Optionally, the method is performed according to a therapy cycle comprising a ramp interval of 5 seconds, a hold interval of 20 seconds, and a deflation interval of 5 seconds.

The present specification also discloses a catheter, comprising: an elongate catheter shaft having a distal region; an inner inflatable member carried by the distal region and defining an inner chamber configured to receive a working fluid; an outer inflatable member carried by the distal region and disposed radially outward of the inner inflatable member, the outer inflatable member defining an outer chamber; and a thermal energy source thermally coupled to the working fluid to heat the working fluid; wherein the inner inflatable member is configured to be inflated to a dilation pressure to dilate a body lumen, and the outer inflatable member is configured to be inflated to a non-dilation pressure that is insufficient to dilate the body lumen; wherein, when the catheter is positioned adjacent tissue and the inner inflatable member contains heated working fluid, the outer inflatable member is selectively configurable between: a contact state in which a first region of the outer inflatable member is in thermal-transmission contact with the tissue to define an ablation zone, and a non-contact state in which a second region of the outer inflatable member is spaced from the tissue to define an insulation zone having reduced heat transfer relative to the ablation zone.

Optionally, the dilation pressure is greater than or equal to 2 atm and the non-dilation pressure is less than 2 atm.

Optionally, the outer inflatable member is configurable to provide the contact state and the non-contact state simultaneously around a circumference of the outer inflatable member.

Optionally, the catheter further comprises a controller configured to adjust at least one of: a pressure or a volume of the outer chamber, a pressure or a volume of the inner chamber, or a temperature or a flow of the working fluid, to modify an extent or location of the ablation zone.

Optionally, the catheter further comprises one or more sensors coupled to the distal region, wherein the one or more sensors comprises at least one of temperature sensor, a pressure sensor, a flow sensor, an impedance sensor, or a contact sensor, wherein the controller regulates the thermal energy source based on signals received from the one or more sensors.

Optionally, the thermal energy source comprises at least one of a resistive heater, an inductive heater, a heated-fluid exchanger, a laser/optical heater, or a microwave heater thermally coupled to the working fluid.

Optionally, the outer inflatable member comprises a wall having spatially varying thermal resistance to accentuate the insulation zone.

Optionally, the outer inflatable member includes patterning features that promote localized contact in the contact state and maintain a gap in the non-contact state.

The present specification also discloses a method of thermally treating tissue with an inflatable catheter, comprising: positioning a distal region of the catheter at a target site adjacent the tissue, wherein the catheter comprises an inner inflatable member and an outer inflatable member disposed outward of the inner inflatable member; inflating the inner inflatable member to a dilation pressure sufficient to dilate a body lumen at the target site; introducing a working fluid into the inner inflatable member and heating the working fluid to a treatment temperature; inflating the outer inflatable member to a non-dilation pressure insufficient to dilate the body lumen, and setting the outer inflatable member to a first inflation condition that establishes a gap between at least a portion of the outer inflatable member and the tissue to form an insulating region; while maintaining the outer inflatable member at the non-dilation pressure, changing at least one inflation parameter of at least one inflatable member to establish contact between at least another portion of the outer inflatable member and the tissue to form a treatment region; and delivering thermal energy from the heated working fluid through the outer inflatable member while the treatment region is in contact with the tissue, thereby preferentially heating tissue in the treatment region relative to tissue adjacent the insulating region.

Optionally, the dilation pressure is greater than or equal to 2 atm and the non-dilation pressure is less than 2 atm.

Optionally, changing the inflation parameter comprises changing pressure, volume, compliance, or shape of the outer inflatable member.

Optionally, the method further comprises cycling between the gap and contact conditions to manage at least one of blood protection, heat-sink control, tissue cooling, or ischemia reduction.

Optionally, the method further comprises sensing at least one of a distal temperature, pressure, flow, contact, or impedance, and controlling heating or inflation in response to the sensing.

Optionally, the insulating region corresponds to a region adjacent blood flow and the treatment region corresponds to tissue targeted for ablation.

The present specification also discloses a treatment assembly, comprising: a catheter including a distal region having an inner inflatable member defining an inner chamber configured to receive a working fluid and an outer inflatable member disposed outward of the inner inflatable member and defining an outer chamber; a console comprising a controller and a user interface; a thermal subsystem coupled to the catheter and configured to heat the working fluid for delivery to the inner chamber; and a fluid management subsystem coupled to the catheter and configured to control at least one of pressure, volume, or flow of at least one of the working fluid delivered to the inner chamber or a fluid delivered to the outer chamber; wherein the controller is configured to cause the fluid management subsystem to set the outer chamber to a first inflation condition establishing a gap between at least a portion of the outer inflatable member and tissue to form an insulating region, and to adjust at least one inflation parameter of at least one of the inner chamber or the outer chamber to establish contact between at least another portion of the outer inflatable member and the tissue to form a treatment region, and wherein the controller is configured to control the thermal subsystem to deliver thermal energy to the tissue through the outer inflatable member while the treatment region is in contact with the tissue.

Optionally, the controller is configured to maintain the outer chamber at a non-dilation pressure while adjusting the at least one inflation parameter to establish the treatment region.

Optionally, the controller is configured to inflate the inner inflatable member to a dilation pressure sufficient to dilate a body lumen and to inflate the outer inflatable member to a non-dilation pressure insufficient to dilate the body lumen during delivery of the thermal energy.

Optionally, the treatment assembly further comprises one or more sensors coupled to the catheter, wherein the one or more sensors comprises a temperature sensor, a pressure sensor, a flow sensor, an impedance sensor, or a contact sensor, wherein the controller is configured to regulate at least one of the thermal subsystem or the fluid management subsystem based on signals received from the one or more sensors.

Optionally, the fluid management subsystem comprises at least one pump, at least one reservoir, and at least one valve configured to independently control the inner chamber and the outer chamber.

Optionally, the controller is configured to cycle the outer chamber between the first inflation condition establishing the gap and a second inflation condition establishing contact to modulate heat transfer to tissue.

In one embodiment, the present specification discloses a device for treating vascular stenosis, comprising: a catheter with a proximal end and a distal end. The catheter includes a first lumen extending from the proximal end to a first point positioned before the distal end; a second lumen extending from the proximal end to a second point at or before the distal end and configured to receive a guidewire; a third lumen extending from the proximal end to the first point positioned before the distal end; a first balloon positioned between said first point and said second point and in fluid communication with the first lumen; a second balloon (inner balloon) positioned within the first balloon (outer balloon) and in fluid communication with the third lumen; and at least one electrode configured to receive a current and positioned in fluid communication with the third lumen and proximate the second balloon. The catheter is in electrical and fluid communication with a controller. The controller comprises programmatic instructions that, when executed: cause a first fluid to be infused into the outer balloon and inflate the outer balloon, cause a liquid to be infused into the inner balloon and thereby increase a pressure of the inner balloon to a first pressure value, maintain the first pressure value for a first period of time, deliver current to the at least one electrode to heat the liquid to generate, or maintain, heated liquid adapted to deliver ablative energy for a second period of time while maintaining the first pressure value for the first period of time, wherein the second period of time is different from the first period of time, stop the delivery of ablative energy via the heated liquid after the second period of time, and remove the liquid from the inner balloon to deflate the inner balloon at the end of the first period of time. Optionally, the second period of time is less than the first period of time.

Optionally, the catheter further comprises a chamber positioned within the inner balloon and the at least one electrode is positioned within the chamber. Optionally, the third lumen is configured to deliver the liquid to the chamber. Optionally, the third lumen is divided into a first subdivision lumen configured to deliver the liquid to the chamber and a second subdivision lumen configured to transfer the liquid away from the chamber.

Optionally, the second balloon comprises a plurality of longitudinal extensions extending from a central axis of the second balloon, wherein each of the plurality of longitudinal extensions is configured to contact an inner surface of the first balloon to create an ablation zone for the transfer of ablative energy at each point of contact.

Optionally, the device further comprises an expandable wire mesh at the distal end of the catheter. Optionally, the expandable wire mesh includes insulation positioned over at least a portion of the wire mesh to prevent the contact of ablative agent with non-target tissue.

Optionally, the at least one electrode comprises at least three layers and at least a portion of the at least one electrode is insulated.

Optionally, the first fluid is any one of air, CO₂, saline or contrast.

Optionally, the second fluid is saline or contrast.

In another embodiment, the present specification discloses a method of treating vascular stenosis, comprising: positioning a distal end of a catheter proximate a stenosed vascular tissue, wherein the catheter comprises: a first lumen extending from a proximal end to a first point positioned before a distal end; a second lumen extending from the proximal end to a second point at or before the distal end and configured to receive a guidewire; a third lumen extending from the proximal end to the first point positioned before the distal end; a first balloon positioned between said first point and said second point and in fluid communication with the first lumen; and a second balloon (inner balloon) positioned within the first balloon (outer balloon) and in fluid communication with the third lumen; and at least one electrode configured to receive a current and positioned in fluid communication with the third lumen and proximate the second balloon, activating a controller to cause: a first fluid to be infused into the outer balloon and inflate the outer balloon, cause a second fluid to be infused into the inner balloon and thereby increase a pressure of the inner balloon to a first pressure value, maintain the first pressure value for a first period of time; deliver current to the at least one electrode to convert the second fluid to heated fluid for ablative energy for a second period of time while maintaining the first pressure value for the first period of time, wherein the second period of time is less than the first period of time; stop the delivery of ablative energy after the second period of time; and remove the second fluid from the inner balloon to deflate the inner balloon at the end of the first period of time.

Optionally, the catheter further comprises a chamber positioned within the inner balloon wherein the at least one electrode is positioned within the chamber.

Optionally, the third lumen is configured to deliver the second fluid to the chamber. Optionally, the third lumen is divided into a first subdivision lumen configured to deliver the second fluid to the chamber and a second subdivision lumen configured to deliver the second fluid away from the chamber.

Optionally, the second balloon comprise a plurality of longitudinal extensions extending from a central axis of the second balloon, wherein each of the plurality of longitudinal extensions is configured to contact an inner surface of the first balloon to create an ablation zone for the transfer of ablative energy at each point of contact.

Optionally, the catheter further comprises an expandable wire mesh at the distal end of the catheter. Optionally, the expandable wire mesh includes insulation positioned over at least a portion of the wire mesh to prevent the contact of ablative agent with non-target tissue.

Optionally, the at least one electrode comprises at least three layers wherein at least a portion of the at least one electrode is insulated.

Optionally, the first fluid is any one of air, CO₂, or contrast.

Optionally, the second fluid is saline.

In another embodiment, the present specification discloses devices for treating vascular stenosis by combining thermal ablative energy and acoustic pressure waves, comprising: a catheter with a proximal end and a distal end. The catheter includes a first lumen extending from the proximal end to a first point positioned before the distal end; a second lumen extending from the proximal end to a second point at or before the distal end and configured to receive a guidewire; a third lumen extending from the proximal end to the first point positioned before the distal end; a first balloon positioned between said first point and said second point and in fluid communication with the first lumen; a second balloon (inner balloon) positioned within the first balloon (outer balloon) and in fluid communication with the third lumen; and at least one electrode configured to receive a current and positioned in fluid communication with the third lumen and proximate the second balloon. Acoustic emitters are integrated into the distal end of the catheter. The catheter is in electrical and fluid communication with a controller. The controller comprises programmatic instructions that, when executed: cause a first fluid to be infused into the outer balloon and inflate the outer balloon. The outer balloon fill fluid (first fluid) preferably comprises an acoustic coupling fluid selected to transmit lithotripsy energy from one or more of the emitters through the outer balloon wall and into calcified tissue. In such embodiments, the acoustic coupling fluid may comprise saline, water, contrast media, or mixtures thereof, and the controller may adjust the acoustic coupling fluid volume, pressure, and/or composition to control acoustic attenuation, coupling efficiency, and/or spatial distribution of lithotripsy energy, while also maintaining thermal insulation and/or cooling functions as described herein. The controller further causes a liquid to be infused into the inner balloon and thereby increase a pressure of the inner balloon to a first pressure value, maintain the first pressure value for a first period of time, and deliver current to the at least one electrode to heat the liquid to generate, or maintain, heated liquid adapted to deliver ablative energy for a second period of time while maintaining the first pressure value for the first period of time. The controller further cooperatively times the delivery of acoustic waves via the one or more emitters with the delivery of ablative energy via the heated liquid. After a predefined dose of acoustic energy and thermal energy, the controller is configured to terminate both the acoustic energy and thermal energy delivery and remove the liquid from the inner balloon to deflate the inner balloon at the end of the first period of time.

In another embodiment, the present specification discloses a method for combining thermal balloon angioplasty therapy and intra-vascular lithotripsy therapy using a single catheter configuration, the method comprising: positioning a distal end of a double-balloon catheter proximal to a target site within a vascular lumen of a patient, wherein the distal end of the double-balloon catheter comprises a first inner balloon of a first diameter, and a second outer balloon of a second diameter greater than the first diameter and placed coaxially outside the inner balloon; inflating the inner balloon with an ablative liquid such as saline or saline-contrast mixture to a first volume; inflating the outer balloon with an acoustic coupling fluid such as saline solution or saline-contrast mixture to a second volume within a space between the inner balloon and the outer balloon; delivering thermal energy to the target site through the saline solution in the inner balloon; delivering acoustic pressure generating pulses to the target site through the acoustic coupling fluid such as saline-contrast mixture solution in the outer balloon; stopping the delivering of thermal energy; deflating the inner balloon by suctioning the saline solution; stopping the delivering of acoustic pressure generating pulses; and deflating the outer balloon by suctioning the saline-contrast mixture solution.

Optionally, inflating the inner balloon comprises inflating to the first volume that builds a first pressure in a range from 2 atm to 8 atm within the inner balloon.

Optionally, inflating the outer balloon comprises inflating to the second volume that builds a second pressure within the outer balloon, wherein the second pressure is greater than the first pressure by at least 0.1 atm, and wherein the second pressure is adjustable.

Optionally, delivering acoustic pressure generating pulses is performed sequentially with the delivering thermal energy.

Optionally, delivering acoustic pressure generating pulses is performed simultaneously with the delivering thermal energy.

Optionally, delivering thermal energy comprises using at least one RF electrode within a first lumen of the double-balloon catheter, wherein the first lumen is in fluid communication with the inner balloon.

Optionally, delivering thermal energy comprises activating the at least one RF electrode to increase a temperature of the saline solution to reach between 60° C. to 65° C. within a duration ranging from 5 seconds to 10 seconds.

Optionally, delivering thermal energy comprises maintaining the temperature range of 60° C. to 65° C. for a duration ranging from 10 seconds to 60 seconds.

Optionally, delivering acoustic pressure generating pulses comprises using an emitter to generate pulses that deliver acoustic pressure at a frequency of 1 Hz.

Optionally, the inflated inner balloon is in contact with the outer balloon during the delivering of the thermal energy and during the delivering of the acoustic pressure generating pulses.

Optionally, the inflated balloon is not in contact with the outer balloon during the delivering of the thermal energy and during the delivering of the acoustic pressure generating pulses.

In some embodiments, the present specification is directed towards a device for combining thermal balloon angioplasty therapy and intra-vascular lithotripsy therapy, comprising: a catheter with a first lumen and a second lumen; an inflatable inner balloon positioned at a distal end of the catheter, the inflatable inner balloon in fluid communication with the first lumen to receive a saline solution, wherein the inflatable inner balloon has a first diameter when maximally inflated; an inflatable outer balloon positioned at the distal end of the catheter, the inflatable outer balloon in fluid communication with the second lumen to receive a saline-contrast mixture solution, wherein the outer balloon is coaxial to the inner balloon, and wherein the outer balloon has a second diameter greater than the first diameter; at least one RF electrode positioned within the first lumen to heat the saline solution for delivering thermal energy through the saline solution in the inflatable inner balloon; at least one emitter positioned within the second lumen to generate pulses for delivering acoustic pressure through the saline-contrast mixture solution in the inflatable outer balloon; and a controller in electrical communication with the at least one RF electrode and the at least one emitter, wherein the controller is configured to control at least one or more of: the delivering of the thermal energy, the delivering of the acoustic pressure pulses, and a volume of the saline-contrast mixture solution in the inflatable outer balloon.

Optionally, the catheter further comprises at least one sensor.

Optionally, the at least one sensor is configured to monitor contact of the inner balloon with the outer balloon or configured to monitor a temperature or pressure of the outer balloon or a temperature or pressure of the inner balloon.

Optionally, upon inflation, the inflatable inner balloon and the inflatable outer balloon each have elongated cylindrical shapes.

The aforementioned and other embodiments of the present specification shall be described in greater depth in the drawings and detailed description provided below.

### BRIEF DESCRIPTION OF THE DRAWINGS

The accompanying drawings illustrate various embodiments of systems, methods, and embodiments of various other aspects of the disclosure. Any person with ordinary skills in the art will appreciate that the illustrated element boundaries (e.g. boxes, groups of boxes, or other shapes) in the figures represent one example of the boundaries. It may be that in some examples one element may be designed as multiple elements or that multiple elements may be designed as one element. In some examples, an element shown as an internal component of one element may be implemented as an external component in another and vice versa. Furthermore, elements may not be drawn to scale. Non-limiting and non-exhaustive descriptions are described with reference to the following drawings. The components in the figures are not necessarily to scale, emphasis instead being placed upon illustrating principles.

FIG. 1A is a representation of a normal blood vessel;

FIG. 1B is an image representation of atherosclerotic stenosis in a blood vessel;

FIG. 2 illustrates a series of representative drawings showing the angioplasty and stenting procedure;

FIG. 3A illustrates a sectional view of a catheter assembly of a device for treating in-stent restenosis and vascular stenosis, in accordance with some embodiments of the present specification;

FIG. 3B illustrates a cross-sectional view along an axis A-A of the catheter assembly shown in FIG. 3A;

FIG. 3C illustrates a system for treating in-stent restenosis and vascular stenosis having the catheter assembly shown in FIG. 3A;

FIG. 4A illustrates a cross-sectional view of an exemplary embodiment of a catheter shaft, which can be used in accordance with some embodiments of the present specification;

FIG. 4B illustrates a cross-sectional view of another exemplary embodiment of a catheter shaft, which can be used in accordance with some embodiments of the present specification;

FIG. 5A illustrates a side view of an exemplary embodiment of an inner balloon of a device for treating in-stent restenosis and vascular stenosis, in accordance with some embodiments of the present specification;

FIG. 5B illustrates a side view of an exemplary embodiment of an outer balloon of a device for treating in-stent restenosis and vascular stenosis, in accordance with some embodiments of the present specification;

FIG. 5C illustrates a side view of another exemplary embodiment of an inner balloon of a device for treating in-stent restenosis and vascular stenosis, in accordance with some embodiments of the present specification;

FIG. 6A is a perspective view of one embodiment of a manifold positioned at the proximal end of the catheter;

FIG. 6B is a side view of one embodiment of a manifold positioned at the proximal end of the catheter;

FIG. 6C is a side view of one embodiment of a manifold positioned at the proximal end of the catheter with conductive wires and a coupled strain relief;

FIG. 7 is a side view of the distal end of one catheter embodiment;

FIG. 8 is a side view of one embodiment of the catheter extending from the proximal manifold to the distal tip;

FIG. 9 illustrates an exemplary embodiment of a distal end of a catheter of a device for treating in-stent restenosis vascular stenosis, in accordance with some embodiments of the present specification;

FIG. 10A is a table listing heating times for saline with different balloon diameters and volumes and various heating electrode power settings, in accordance with some embodiments of the present specification;

FIG. 10B is another table listing heating times for saline with different balloon diameters and volumes and various heating electrode power settings, in accordance with some embodiments of the present specification;

FIG. 11A illustrates an exemplary design of an electrode configuration, used for heating an ablation fluid for hydrothermal ablation, in accordance with some embodiments of the present specification;

FIG. 11B separately illustrates the different layers used to form the electrode configuration of FIG. 11A, in accordance with some embodiments of the present specification;

FIG. 12 illustrates an exemplary sequence of steps of thermal balloon angioplasty while a catheter is positioned within an artery with a stent and ISR, in accordance with some embodiments of the present specification;

FIG. 13 illustrates an exemplary sequence of steps of thermal balloon angioplasty while a catheter is positioned within an artery with vascular stenosis (VS), in accordance with some embodiments of the present specification;

FIG. 14 is a flow chart illustrating the fundamental sequence of steps to achieve thermal angioplasty to treat atherosclerotic VS, in accordance with an embodiment of the present specification;

FIG. 15 is a flow chart illustrating another exemplary sequence of steps to achieve thermal angioplasty to treat atherosclerotic VS, in accordance with an embodiment of the present specification;

FIG. 16 is a flow chart illustrating another exemplary sequence of steps to achieve thermal angioplasty to treat atherosclerotic VS, in accordance with an embodiment of the present specification;

FIG. 17 is a flow chart illustrating another exemplary sequence of steps to achieve thermal angioplasty to treat atherosclerotic VS, in accordance with an embodiment of the present specification;

FIG. 18 is a flow chart illustrating yet another exemplary sequence of steps to achieve thermal angioplasty to treat atherosclerotic VS, in accordance with an embodiment of the present specification;

FIG. 19 is a flow chart illustrating an exemplary sequence of steps to achieve thermal angioplasty to treat ISR, in accordance with an embodiment of the present specification;

FIG. 20 is a flow chart illustrating another exemplary sequence of steps to achieve thermal angioplasty to treat ISR, in accordance with an embodiment of the present specification;

FIG. 21 is a flow chart illustrating another exemplary sequence of steps to achieve thermal angioplasty to treat ISR, in accordance with an embodiment of the present specification;

FIG. 22 is a flow chart illustrating another exemplary sequence of steps to achieve thermal angioplasty to treat ISR, in accordance with an embodiment of the present specification;

FIG. 23 is a flow chart illustrating another exemplary sequence of steps of thermal ablation to treat atherosclerotic VS, in accordance with an embodiment of the present specification;

FIG. 24 is a flow chart illustrating another exemplary sequence of steps of thermal ablation to treat ISR, in accordance with an embodiment of the present specification;

FIG. 25 is a flow chart illustrating yet another exemplary sequence of steps of thermal ablation to treat ISR, in accordance with an embodiment of the present specification;

FIG. 26 is a flow chart that illustrates an exemplary process of an integrated TBA and IVL procedure using a dual mode catheter, in accordance with some embodiments of the present specification;

FIG. 27A illustrates an inner balloon filled with saline positioned coaxially around a guide sheath within an outer balloon filled with 50/50 saline contrast mix, in accordance with some embodiments of the present specification;

FIG. 27B illustrates heated inner balloon which, in its expanded (inflated) form, contact outer balloon to create a hot ablation zone, in accordance with some embodiments;

FIG. 27C illustrates a gap arising due to non-contact between surfaces of inner balloon and outer balloon, in accordance with some embodiments of the present specification;

FIG. 27D illustrates a hybrid mode achieved by alternatively cycling between the thermal ablation mode and the IVL mode or by applying IVL simultaneously with thermal energy, in accordance with some embodiments of the present specification;

FIG. 28A illustrates longitudinal and transverse perspective views of a dual balloon cardiac ablation catheter, in accordance with some embodiments of the present specification;

FIG. 28B illustrates a longitudinal cross-sectional view along with an enlarged view of a portion of the catheter of FIG. 25A, in accordance with some embodiments of the present specification;

FIG. 28C illustrates a transverse cross-sectional view of an elongate body of the catheter of FIG. 28A, in accordance with some embodiments of the present specification;

FIG. 28D illustrates transverse cross-sectional, longitudinal cross-sectional and perspective views of an outer catheter of the catheter of FIG. 25A, in accordance with some embodiments of the present specification;

FIG. 28E illustrates transverse and longitudinal cross-sectional views of an inner catheter of the catheter of FIG. 28A, in accordance with some embodiments of the present specification;

FIG. 28F illustrates transverse and longitudinal cross-sectional views of a cooling tube of the catheter of FIG. 28A, in accordance with some embodiments of the present specification;

FIG. 28G illustrates transverse and various perspective views of an outer balloon of the catheter of FIG. 28A, in accordance with some embodiments of the present specification;

FIG. 28H illustrates transverse and various perspective views of an inner balloon of the catheter of FIG. 28A, in accordance with some embodiments of the present specification;

FIG. 28I illustrates an angular perspective view, a side perspective view and a longitudinal cross-sectional view of a bulbous or olive tip, in accordance with an embodiment of the present specification;

FIG. 29A details a first administration protocol of both ablative energy and acoustic energy;

FIG. 29B details a second administration protocol of both ablative energy and acoustic energy;

FIG. 29C details a third administration protocol of both ablative energy and acoustic energy;

FIG. 30 details a functional relationship between power availability and impedance; and

FIG. 31 shows a controller closed loop feedback architecture.

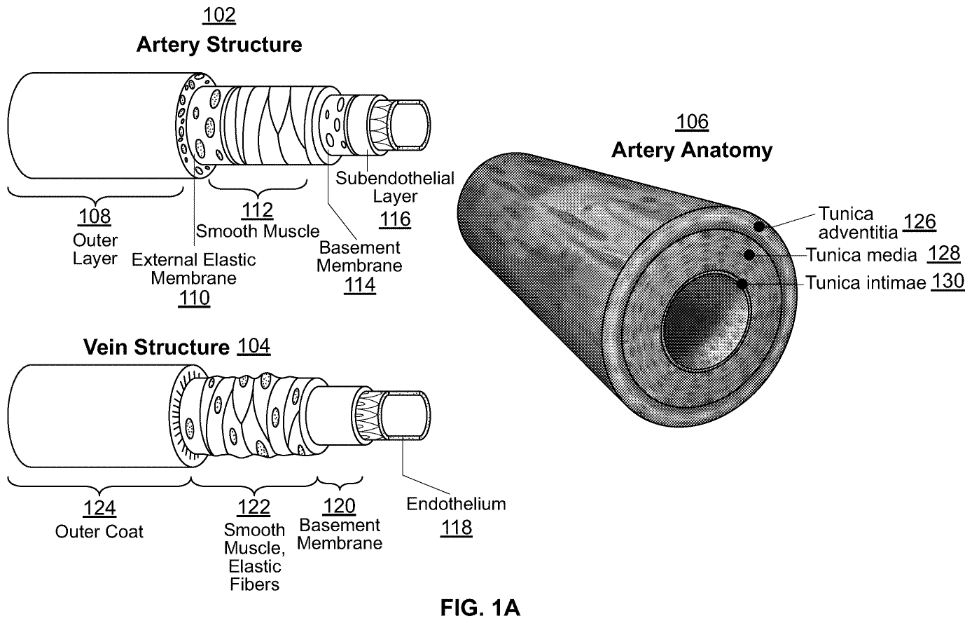

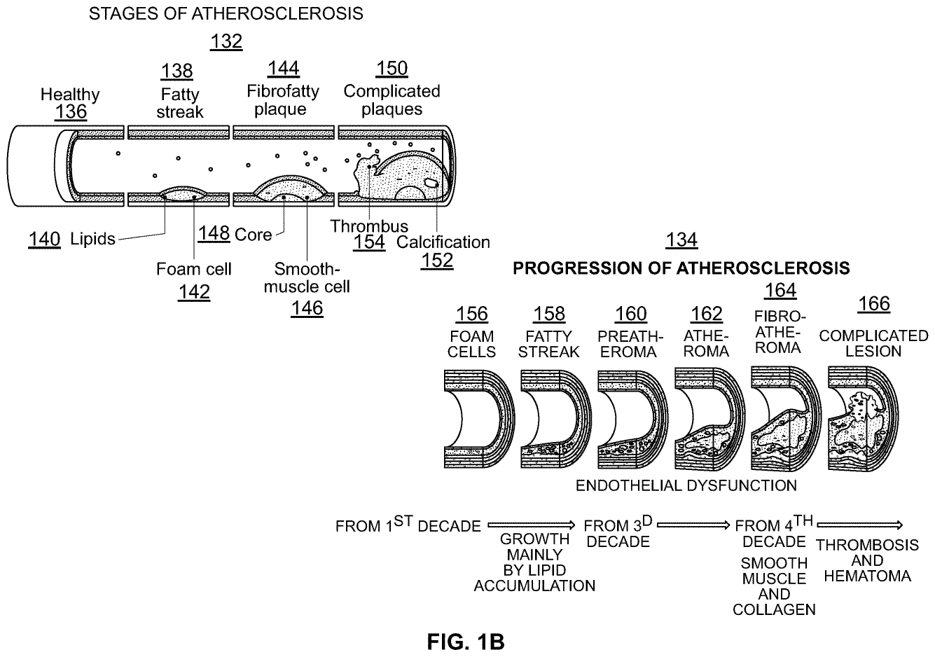

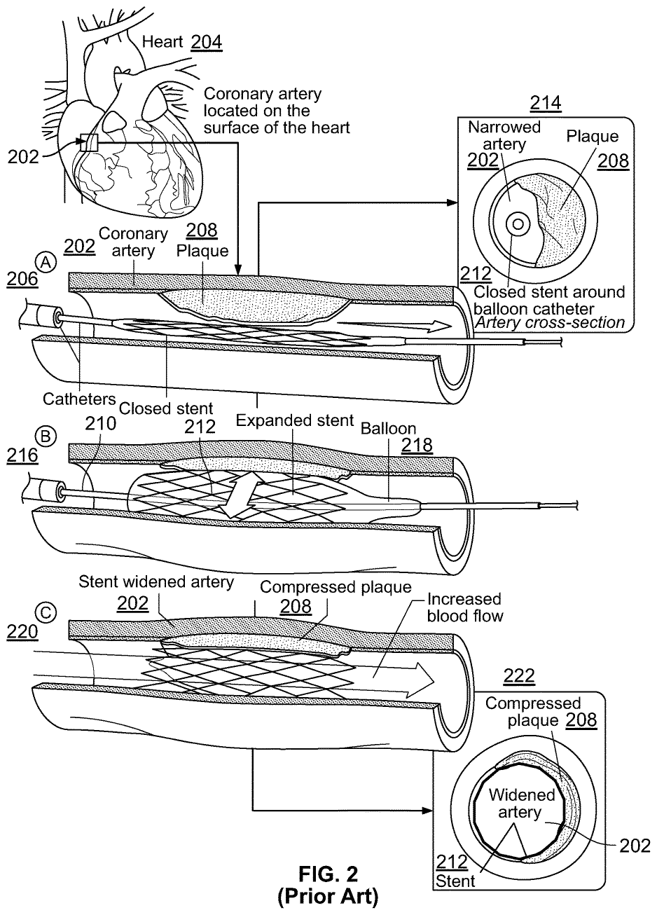

### DETAILED DESCRIPTION

The present specification is directed toward multiple embodiments for methods and devices that are used for endothelial rejuvenation therapy to treat atherosclerotic vascular disease or in-stent restenosis (ISR) and simultaneously prevent or reduce the recurrence of the disease. Endothelial rejuvenation, also referred to as intimal or vascular rejuvenation, is a procedure where the diseased intima or media are ablated resulting in cell necrosis and healing, wherein the diseased intima or media do not have the inherent abnormalities of the diseased intima or media following treatment. The neointima or neo-endothelium are less prone to atherosclerosis, hyperplasia, dysplasia or restenosis.

In an embodiment, at least 20% of the diseased intima or media is ablated to achieve the desired therapeutic objective. In another embodiment >50% of the diseased intima or media is ablated to achieve the desired therapeutic objective. In another embodiment, the diseased intima or media showing the most structural changes and restenosis is selectively ablated compared to the intima or media showing the least structural changes and restenosis to achieve the desired therapeutic objective.

The desired therapeutic objective in patients with primary stenosis, restenosis and ISR includes an increase in the lumen diameter from <30% to >50% in a patient (per-patient success). Another desired therapeutic objective is that >50% patients achieve a lumen diameter of >50% immediately after the intervention (immediate success). Another desired therapeutic objective is that >50% patients achieve immediate success and are able to maintain the success for 1 year (intermediate success). Another desired therapeutic objective is that >50% patients achieve intermediate success and maintain the success for 5-years (delayed success).

Yet another desired therapeutic objective is an increase in relative arterial cross-sectional area by at least 10% with a hot inflation, compared to similar therapy using only cold inflation.

Yet another desired therapeutic objective is an increase in relative medial wall thinning by at least 10% with a hot inflation, compared to similar therapy using only cold inflation.

Yet another desired therapeutic objective in patients with primary stenosis, restenosis and ISR includes a decrease in time to re-epithelialization by at least 10% with hot inflation, compared to similar therapy using only cold inflation, and hence reducing the duration of antiplatelet therapy by at least 10% compared to a similar therapy without use of thermal ablation.

Another desired therapeutic objective in patients with primary stenosis, restenosis and ISR includes a relative decrease in reinterventions by at least 10% with hot inflation compared to similar therapy using only cold inflation, thereby reducing morbidity, mortality and cost associated with these conditions.

Another desired therapeutic objective in patients with primary stenosis, restenosis and ISR includes a relative decrease in symptomatic recurrences by at least 10% with hot inflation compared to a similar therapy using only cold inflation, thereby reducing morbidity, mortality and cost associated with these conditions.

Another desired therapeutic objective in patients with primary stenosis, restenosis and ISR includes a decrease in the grade of stenosis or restenosis by at least 1 grade compared to a similar therapy without use of thermal ablation.

Another desired therapeutic objective in patients with primary stenosis and restenosis includes thickening of the fibrous cap of atherosclerotic plaques, potentially promoting plaque stability with hot inflation compared to similar therapy using only cold inflation.

Thermal ablation, as used in the process of vascular rejuvenation, may include one of different types of modalities, such as hydrothermal ablation (HTA) including heated liquid, cryoablation, radiofrequency (RF) ablation, microwave (MW) ablation, Electroporation (EP), High Intensity Focused Ultrasound (HIFU) ablation, or any other ablation modality that achieves one or more of the stated therapeutic objectives. The thermal ablation modalities can be used alone or in combination with another modality.

The present specification is also directed toward enhancing the efficacy of calcified plaque treatment through the sequential and/or simultaneous application of thermal energy and acoustic shock waves. In various embodiments, methods and devices of the present specification leverage thermal energy and acoustic pressure waves to achieve superior outcomes in the treatment of calcified vascular plaques. Thermal balloon angioplasty methods and systems are used in combination with intravascular lithotripsy (IVL) methods and systems to address longstanding limitations in angioplasty procedures, offering enhanced vessel compliance, reduced restenosis rates, and improved procedural efficiency in vascular interventions. Embodiments of the present specification deliberately and safely apply thermal energy in conjunction with IVL while addressing the limitations of both modalities. The dual-modality design of the present specification is validated through experimental models and demonstrates superior performance compared to conventional treatments. Embodiments provide an optimized double-balloon design and precise temperature regulation to achieve an effective and safe vascular intervention tool.

The long-standing industry guidance against heating during IVL therapy is overcome by the integration of thermal balloon angioplasty with IVL in the present specification. The catheter design embodiments of the present specification deliberately and safely apply thermal energy in conjunction with IVL, thereby challenging the assumptions of the existing methods and addressing the limitations of both modalities.

Embodiments of the present specification provide a hybrid catheter device and methods of its use, which combines thermal balloon angioplasty (TBA) and IVL technologies. The combination device enables either simultaneous or sequential application of thermal energy and acoustic pressure waves, while improving safety, efficacy and usability of the device. The dual-modality approach of the combination device effectively fractures, softens, and remodels calcified plaques, resulting in improved vascular compliance, reduced elastic recoil, and lower restenosis rates.

The present specification is directed towards multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For the purpose of clarity, details relating to technical material that is known in the technical fields related to the invention have not been described in detail so as not to unnecessarily obscure the present invention.

“Treat,” “treatment,” and variations thereof refer to any reduction in the extent, frequency, or severity of one or more symptoms or signs associated with a condition.

“Duration” and variations thereof refer to the time course of a prescribed treatment, from initiation to conclusion, whether the treatment is concluded because the condition is resolved or the treatment is suspended for any reason. Over the duration of treatment, a plurality of treatment periods may be prescribed during which one or more prescribed stimuli are administered to the subject.

“Period” refers to the time over which a “dose” of stimulation is administered to a subject as part of the prescribed treatment plan.

As used herein, the term “fluid” includes liquids, gases, vapors, and mixtures thereof. For example, fluid may comprise an aqueous solution such as saline, sterile water, dextrose solution (e.g., D5W or other glucose-containing solutions), buffered solutions (e.g., phosphate-buffered solutions, bicarbonate-buffered solutions), balanced electrolyte solutions (e.g., lactated Ringer's, Plasma-Lyte®), or any combination or mixture thereof. In another example, fluid may further include one or more additives such as radiopaque contrast, dyes, electrolytes, surfactants, anti-foaming agents, anticoagulants (e.g., heparin), anti-proliferative drugs, anesthetics, vasodilators, or other therapeutic agents, provided that the fluid remains biocompatible for intravascular use. Accordingly, references herein to “saline” or “heated saline”, when used in the context of “fluid”, are exemplary only, and in various embodiments an ablative fluid and/or inflation fluid may comprise air, water, saline, dextrose solution, buffered solution, balanced electrolyte solution, or mixtures thereof, and may be delivered and/or generated as a heated liquid, heated vapor, or a two-phase liquid/vapor mixture.

As used herein, the term “dilation pressure” means an inflation pressure applied to an inflatable member, such as a balloon, that, in the intended anatomical environment and under the conditions of use, dilates a body lumen (or other body cavity) such that a lumen dimension increases by 5% or greater relative to a pre-dilation lumen dimension measured at the target site immediately prior to applying the dilation pressure. In some embodiments herein, a value of dilation pressure is within a range from 2 atm to 100 atm, inclusive. It should be appreciated, however, that dilation pressure is not limited to any particular numerical value and may vary with anatomy, lesion compliance, balloon geometry, material properties, temperature, and other clinical factors.

As used herein, the term “non-dilation pressure” means an inflation pressure applied to an inflatable member, such as a balloon, that is insufficient, in the intended anatomical environment and under the conditions of use, to cause dilation of the body lumen as defined herein. A non-dilation pressure may be less than 2 atm, and/or may be any pressure that results in a change in the lumen dimension of less than 5% relative to the pre-dilation lumen dimension. Non-dilation pressure may be used to achieve apposition, contact patterning, spacing, acoustic coupling, insulation, or other functional states without materially enlarging the lumen.

As used herein, the term “supra-dilation pressure” means an inflation pressure applied to an inflatable member, such as a balloon, that is greater than the dilation pressure for the specific application, target site, and conditions of use. Supra-dilation pressure may be defined relative to the dilation pressure determined for the same procedure, anatomy, and catheter configuration, and can be used to increase radial force, modify contact conditions, alter coupling conditions, or otherwise change an interaction between the inflatable member and tissue. Supra-dilation pressure is not limited to a particular numerical value and can exceed the dilation pressure by any amount suitable for the intended effect.

As used herein, the term “lumen dimension” may refer to the diameter, cross-sectional area, circumference, or another suitable geometric measure. A percentage change in the lumen dimension may be determined using imaging, pressure-diameter characterization, or other measurement techniques before and during inflation, as is known to persons of ordinary skill in the art. The foregoing definitions are intended to be context-dependent so that the described pressure regimes are characterized by their effect on the lumen and not by a fixed pressure value, and may be applied to different anatomies, catheter sizes, and use cases without limitation.

The term “and/or” means one or all of the listed elements or a combination of any two or more of the listed elements.

In the description and claims of the application, each of the words “comprise”, “include”, “have”, “contain”, and forms thereof, are not necessarily limited to members in a list with which the words may be associated. Thus, they are intended to be equivalent in meaning and be open-ended in that an item or items following any one of these words is not meant to be an exhaustive listing of such item or items, or meant to be limited to only the listed item or items. It should be noted herein that any feature or component described in association with a specific embodiment may be used and implemented with any other embodiment unless clearly indicated otherwise.

It must also be noted that as used herein and in the appended claims, the singular forms “a,” “an,” and “the” include plural references unless the context dictates otherwise. Although any systems and methods similar or equivalent to those described herein can be used in the practice or testing of embodiments of the present disclosure, the preferred, systems and methods are now described.

The term “controller” refers to an integrated hardware and software system defined by a plurality of processing elements, such as integrated circuits, application specific integrated circuits, and/or field programmable gate arrays, in data communication with memory elements, such as random access memory or read only memory where one or more processing elements are configured to execute programmatic instructions stored in one or more memory elements.

The term “ablative fluid generation system” refers to any or all of the heater or induction-based approaches to generating hot liquid as described in this application.

For purposes of the present specification, ‘completely ablating’ is defined as ablating more than 55% of a surface area or a volume around an anatomical structure.

All of the methods and systems for treating the artery may include optics or visualization as described in the specification to assist with direct visualization during ablation procedures.

All ablation catheters disclosed in the specification, in some embodiments, include insulation at the location of the electrode(s) to prevent ablation of tissue proximate the location of the electrode within the catheter.

For any method disclosed herein that includes discrete steps, the steps may be conducted in any feasible order. And, as appropriate, any combination of two or more steps may be conducted simultaneously.

Also herein, the recitations of numerical ranges by endpoints include all numbers subsumed within that range (e.g., 1 to 5 includes 1, 1.5, 2, 2.75, 3, 3.80, 4, 5, etc.). Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present specification. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the specification are approximations, the numerical values set forth in the specific examples are reported as precisely as possible. All numerical values, however, inherently contain a range necessarily resulting from the standard deviation found in their respective testing measurements.

As described below, the methods, systems, and devices/catheters and various components described in the present specification include key functionalities related to the combination of certain features including a double-balloon structure for providing thermal ablative energy and components directed to simultaneously providing Thermal Balloon Angioplasty (TBA) with acoustic energy and components directed to Intravascular Lithotripsy (IVL) in the treatment of atherosclerotic vascular disease and in-stent restenosis. Key functionalities include, but are not limited to, a multi-lumen catheter having a double balloon positioning element at its distal end, with an inner balloon positioned within an outer balloon, the ability to provide thermal energy to the balloons, particularly the inner balloon for transfer to a target tissue, wherein the thermal energy may be provided in the form of hydrothermal ablation (HTA) including hot water, cryoablative fluid, direct application of radiofrequency (RF) ablative energy (not mediated by a liquid or vapor), direct application of microwave (MW) ablation energy (not mediated by a liquid or vapor), direct application of electroporation (EP) ablative energy (not mediated by a liquid or vapor), high intensity focused ultrasound (HIFU) ablative energy (not mediated by a liquid or vapor), and the ability to provide acoustic pressure waves via the catheter to assist in the atherosclerotic vascular disease/in-stent restenosis treatment. Therefore, the use and combination of these features should not be construed as mere design choices and, rather, should be accorded patentable weight.

It should also be noted that the various components described herein may be used with any other component as described herein, in any combination or order, even if not described with respect to certain embodiments. Further, it should be noted that the various features and parameters described herein may apply without restriction to the embodiments in which they are described. Therefore, the components and parameters described throughout this specification are interchangeable and may be combined to achieve the objectives of the present invention and not limited to the specific embodiments. Further, it should be appreciated that, whenever a range of values is provided, any subset of that range, including one number, or multiple numbers, is being equivalently disclosed.

The devices and methods of the present specification can be used to cause controlled circumferential ablation of targeted tissue to varying depth in a manner in which complete healing with re-epithelialization can occur. Additionally, the ablative energy could be used to treat/ablate calcified plaque deposits. The dose and manner of treatment can be adjusted based on the type of tissue and the depth of ablation needed. The ablation device can be placed endoscopically, radiologically, surgically or under direct visualization. In various embodiments, wireless endoscopes or single fiber endoscopes can be incorporated as a part of the device. In another embodiment, magnetic or stereotactic navigation can be used to navigate the catheter to the desired location. Radio-opaque or sonolucent material can be incorporated into the body of the catheter for radiological localization. Ferromagnetic materials can be incorporated into the catheter to help with magnetic navigation.

Ablative agents such as heated liquids or cryogenic liquid, such as, but not limited to, liquid nitrogen are inexpensive and readily available and are directed via the infusion port onto the tissue, held at a fixed and consistent distance, targeted for ablation. This allows for uniform distribution of the ablative agent on the targeted tissue. The flow of the ablative agent is controlled by a microprocessor according to a predetermined method based on the characteristic of the tissue to be ablated, required depth of ablation, and distance of the port from the tissue. The microprocessor may use temperature, pressure or other sensing data to control the flow of the ablative agent. In addition, one or more suction ports are provided to suction the ablation agent from the vicinity of the targeted tissue. The targeted segment can be treated by a continuous infusion of the ablative agent or via cycles of infusion and removal of the ablative agent as determined and controlled by the microprocessor.

It should be appreciated that the devices and embodiments described herein are implemented in concert with a controller that comprises a microprocessor executing control instructions. The controller can be in the form of any computing device, including desktop, laptop, and mobile device, and can communicate control signals to the ablation devices in wired or wireless form.

It should be noted herein that any feature or component described in association with a specific embodiment may be used and implemented with any other embodiment unless clearly indicated otherwise.

### Catheter Assembly

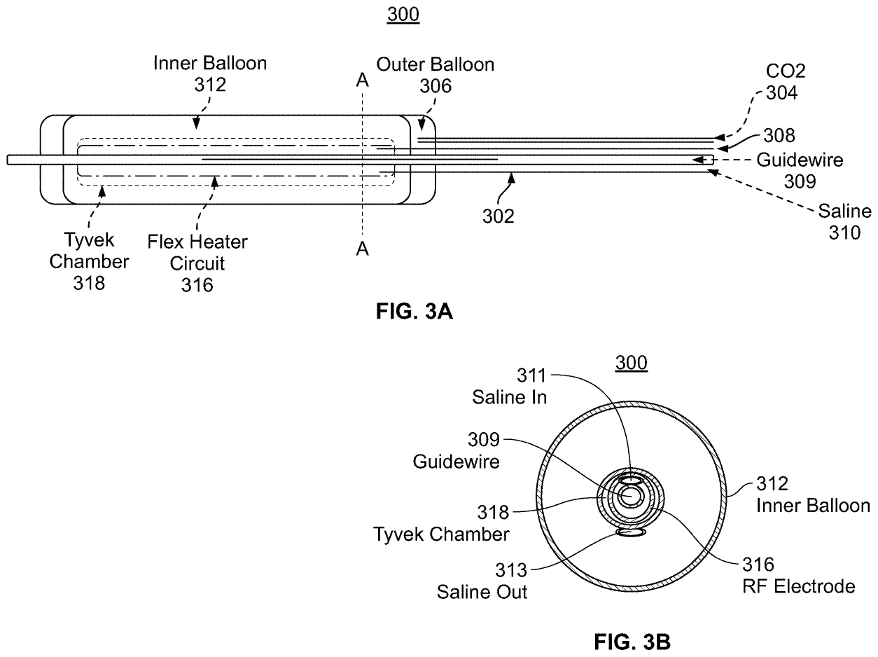

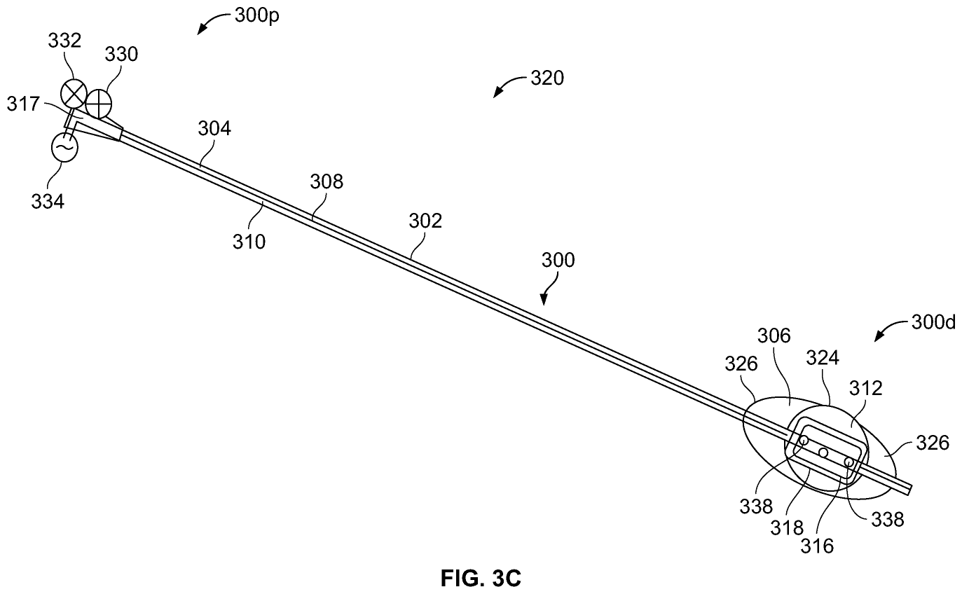

FIG. 3A illustrates a sectional view of a catheter assembly 300 of a device for treating in-stent restenosis, in accordance with some embodiments of the present specification. FIG. 3B illustrates a cross-sectional view along an axis A-A shown in FIG. 3A, of catheter assembly 300. Referring simultaneously, to FIGS. 3A and 3B, a catheter shaft 302 includes multiple parallel lumens. A first lumen 304 provides a channel to deliver fluid including saline/air/carbon dioxide (CO₂) for inflating an outer balloon 306 that is attached to a distal end of shaft 302. The first lumen 304 is in fluid communication with the outer balloon 306. In embodiments, the first lumen 304 extends from a proximal end of the catheter assembly 300 to a first point before a distal end of the catheter assembly 300. A second lumen 308 passes centrally through shaft 302 to provide passage for a guidewire 309. Lumen 308 passes centrally through shaft 302 and through outer balloon 306 and inner balloon 312 to a distal side of balloons 306, 312. In some embodiments, the second lumen 308 extends from a proximal end of the catheter assembly 300 to a second point before or at a distal end of the catheter assembly 300. A third lumen 310 extends within shaft to deliver saline into a chamber 318 within inner balloon 312. The third lumen 310 is in fluid communication with the chamber 318 which, in turn, is in fluid communication with the inner balloon 312. In embodiments, the third lumen 310 extends from a proximal end of the catheter assembly 300 to a first point before a distal end of the catheter assembly 300.

Referring to FIG. 3B, in some embodiments, the third lumen 310 is further divided into first subdivision lumen 311 for saline to enter the chamber 318 and second subdivision lumen 313 for saline to exit the chamber 318. In other embodiments, the saline enters and exits the chamber 318 through the same third lumen 310. A flex heater circuit 316 comprising at least one RF electrode is positioned within the chamber 318 that is positioned within the inner balloon 312. In some embodiments, chamber 318 is composed of PTFE or GORTEX. In these embodiments, the layer of GORTEX or PTFE or a comparable material known in the art serves as a porous layer that allows for passage of the heated liquid but doesn't allow vapor to pass into the inner balloon 312. Chamber 318 is in fluid communication with third lumen 310 and with the inner balloon 312 and is configured to receive saline through third lumen 310 and heat the saline to convert the liquid to heated liquid by heater circuit 316 and deliver the heated liquid to the inner balloon 312. Inner balloon 312 surrounds chamber 318, and is inflated by heated liquid to a diameter so that at least some portions of an outer surface of inner balloon 312 contacts at least some portions of an inner surface of outer balloon 306 during an ablation process, creating an ablation zone to contact tissue.

In some embodiments, chamber 318 does not have the layer of PTFE or GORTEX and the flex circuit directly heats the saline in the inner balloon 312, wherein inner balloon 312 serves the function of the chamber 318. In this embodiment, hot water or saline is the ablative agent.

FIG. 3C illustrates a system 320 for treating in-stent restenosis and vascular stenosis having the catheter assembly shown in FIG. 3A. System 320 comprises a catheter assembly 300 with an inner balloon 312 positioned within an outer balloon 306, in accordance with an embodiment of the present specification. Chamber 318 with a flex heater circuit 316 positioned within the chamber 318, is in turn positioned within the inner balloon 312. In certain embodiments, the inner balloon 312 serves as the chamber 318. Catheter 300 includes an elongate catheter body 302 and has a proximal end 300p and a distal end 300d with a first lumen 304, a second lumen 308, and a third lumen 310 extending therein. In embodiments, the first lumen 304 is a fluid lumen to deliver saline/air/carbon dioxide (CO₂) to provide fluid for inflating the outer balloon 306, and is in in fluid communication with a fluid pump 330 at the proximal end 300p of the catheter 300. In embodiments, the second lumen 308 is a guidewire lumen for passage of a guidewire through the length of the catheter 300. In embodiments, the third lumen 310 is a fluid lumen for receiving saline from a saline pump 332 at the proximal end 300p of the catheter 300.

In the present specification, the terms inner balloon and ablation balloon are used interchangeably. A plurality of infusion ports 338 are included in the chamber 318 and allow for the passage of heater liquid from the chamber 318 into the inner or ablation balloon 312. In certain embodiments the layer of GORTEX or PTFE serves as a porous layer that allows for passage of the heated liquid but doesn't allow for vapor to pass.

The system 320 includes a controller 317 configured to cause the fluid/air/CO₂ pump 330 to deliver fluid, air or CO₂, via the first lumen 304, to the outer balloon 306 to change the outer balloon from a first state, in a compressed, deflated configuration, to a second state, in an expanded, inflated configuration. The controller is also configured to cause the saline pump 332 to deliver water or saline to the third lumen 310 to chamber 318, where flex heater circuit 316 comprising at least one RF electrode is configured to provide thermal energy to the liquid, i.e. water or saline, to convert the liquid to heated liquid. This heated liquid is then delivered via ports 338 or through the porous layer of a material to the inner ablation balloon 312. In embodiments where the inner balloon 312 functions as the chamber 318, the flex heater circuit 316 directly heats the liquid in the balloon 312. In embodiments, the at least RF electrode of the flex heater circuit 316 is configured to receive an RF current from RF generator 334 to heat the electrode and convert the liquid to heated liquid. Heated liquid provided to the ablation balloon causes the ablation balloon to change from a first state, in a compressed deflated configuration, to a second state, in an expanded, inflated configuration. Areas of the outer balloon 306 where the inflated ablation balloon 312 contacts the inflated outer balloon 306 comprise hot or “ablation zones” 324 and provide for the transfer of ablative energy from the ablation balloon 312 to the target tissue for ablation. Non-ablation zones 326, also referred to as cold zones or insulation zones, comprise areas of the outer balloon 306 where the inner balloon 312 and outer balloon 306 do not contact one another and have an average temperature that is different than an average temperature of ablation zones and is in the non-ablation range.

In certain embodiments, a cryoablation agent such as liquid CO₂ or liquid nitrogen is delivered to the inner balloon 312 and the areas of the outer balloon 306 where the cryo-inflated ablation balloon 312 contacts the inflated outer balloon 306 comprise cold or ablation zones 324 and provide for the transfer of ablative energy from the ablation balloon 312 to the target tissue for ablation. Non-ablation zones 326, also referred to as insulation zones, comprise areas of the outer balloon 306 where the inner balloon 312 and outer balloon 306 do not contact one another and have an average temperature that is higher than an average temperature of ablation zones and is in the non-ablation range.

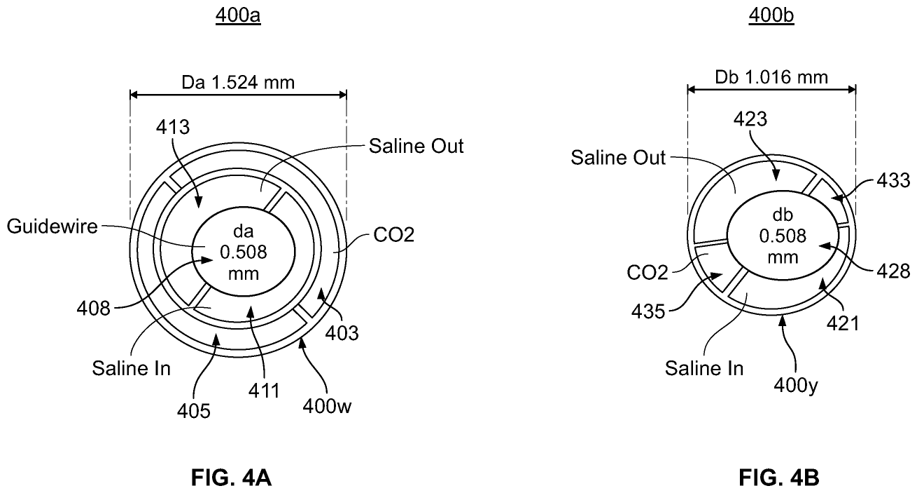

FIG. 4A illustrates a cross-sectional view of an exemplary embodiment of a catheter shaft 400a, which can be used in accordance with some embodiments of the present specification. In embodiments, a diameter Da of shaft 400a is approximately 1.524 mm. A second lumen 408 is configured inside shaft 400a and concentric to shaft 400a, and with a diameter da of approximately 0.508 mm. Second lumen 408 is used to provide passage to a guidewire of approximately 0.406 mm. A first subdivision lumen 411 (of third lumen 310 of FIG. 3A) is configured between and concentric to an outer wall 400w of shaft 400a and second lumen 408 and is configured to deliver saline to the chamber 318 of FIG. 3A. In embodiments, a diameter of first subdivision lumen 411 is less than Da. A second subdivision lumen 413 (of third lumen 310 of FIG. 3A) is configured between and concentric to outer wall 400w of shaft 400a and second lumen 408 and is configured to deliver saline away from the chamber 318 of FIG. 3A. In embodiments, a diameter of second subdivision lumen 413 is less than Da. A third subdivision lumen 403 (of first lumen 304 of FIG. 3A) is configured between and concentric to outer wall 400w of shaft 400a and first and second subdivision lumens 411, 413 and is configured to transport fluid/air/CO₂ to and away from the outer balloon 306 of FIG. 3A. A fourth subdivision lumen 405 (of first lumen 308 of FIG. 3A) is configured between and concentric to outer wall 400w of shaft 400a and first and second subdivision lumens 411, 413 and is configured to transport fluid/air/CO₂ to and away from the outer balloon 306 of FIG. 3A.

FIG. 4B illustrates a cross-sectional view of another exemplary embodiment of a catheter shaft 400b, which can be used in accordance with some embodiments of the present specification. A diameter Db of shaft 400b is approximately 1.016 mm. A second lumen 428 is configured inside shaft 400b and concentric to shaft 400b, and with a diameter db of approximately 0.508 mm. Second lumen 428 is used to provide passage to a guidewire of approximately 0.406 mm. A first subdivision lumen 421 (of third lumen 310 of FIG. 3A) is configured between and concentric to an outer wall 400y of shaft 400b and second lumen 428 and is configured to deliver saline to the chamber 318 of FIG. 3A. In embodiments, a diameter of first subdivision lumen 421 is less than Db. A second subdivision lumen 423 (of third lumen 310 of FIG. 3A) is configured between and concentric to outer wall 400y of shaft 400a and second lumen 428 and is configured to deliver saline away from the chamber 318 of FIG. 3A. In embodiments, a diameter of second subdivision lumen 423 is less than Da. A third subdivision lumen 433 (of first lumen 304 of FIG. 3A) is configured between and concentric to outer wall 400w of shaft 400a and second lumen 428 and radially adjacent to first and second subdivision lumens 421, 421, and is configured to transport fluid/air/CO₂ to and away from the outer balloon 306 of FIG. 3A. A fourth subdivision lumen 435 (of first lumen 308 of FIG. 3A) is configured between and concentric to outer wall 400w of shaft 400a and second lumen 428 and radially adjacent to first and second subdivision lumens 421, 421, and is configured to transport fluid/air/CO₂ to and away from the outer balloon 306 of FIG. 3A.

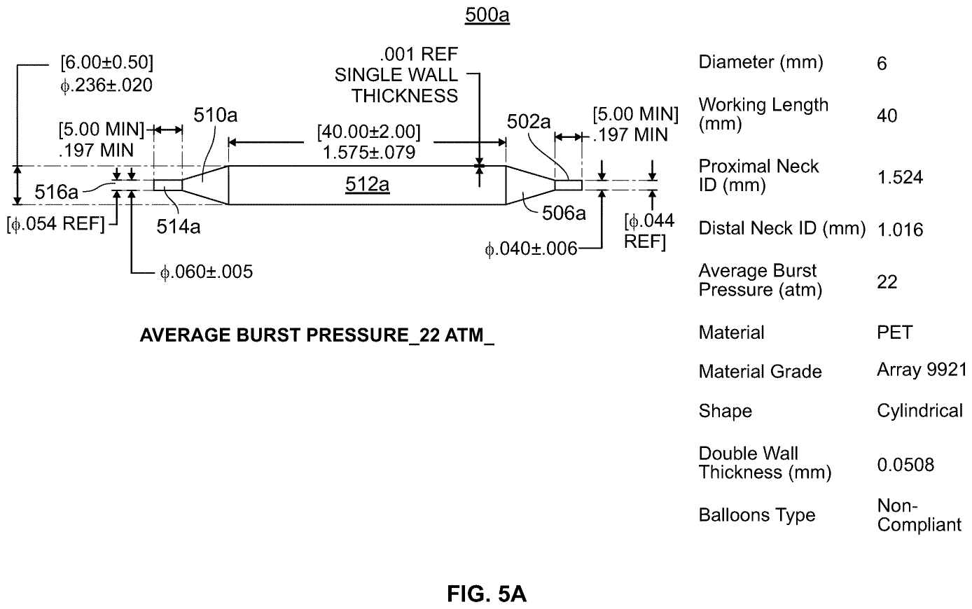

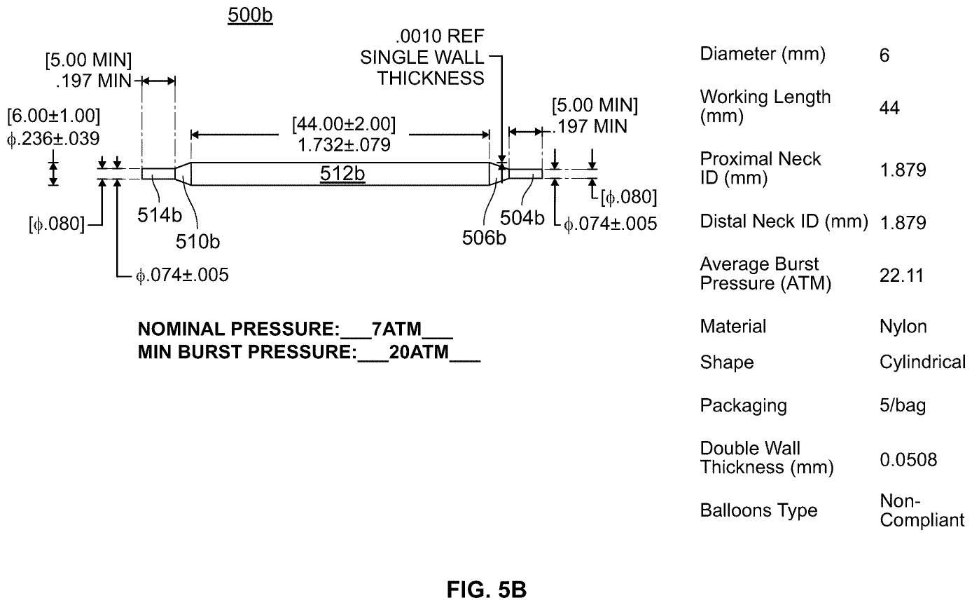

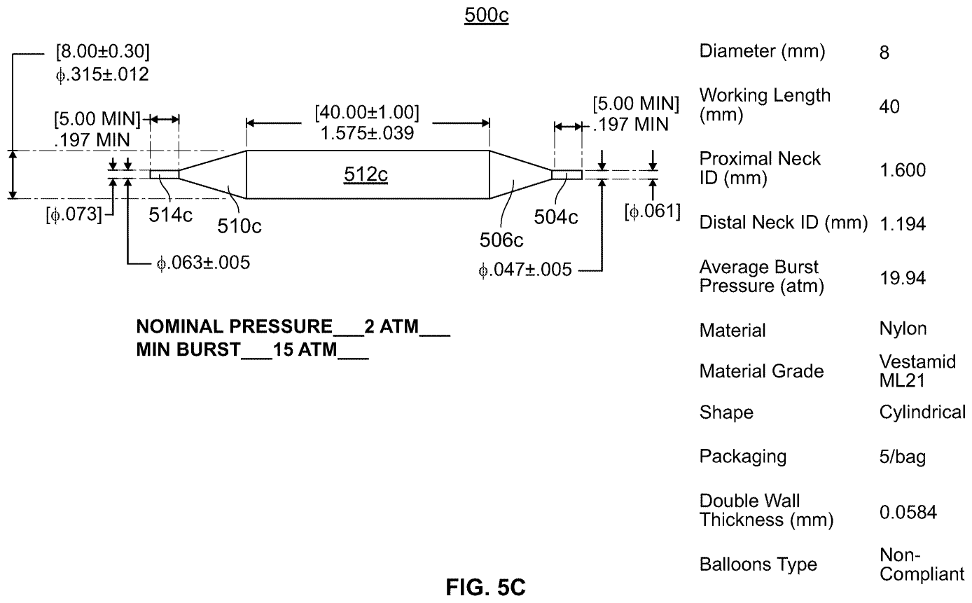

FIG. 5A illustrates a side view of an exemplary embodiment of an inner balloon 500a of a device for treating in-stent restenosis and vascular stenosis, in accordance with some embodiments of the present specification. A distal neck 502a of the inner balloon 500a has a diameter in a range of 1.016±0.127 mm. Distal neck 502a extends toward and connects at its proximal end to a distal conical portion 506a of the inner balloon 500a. A balloon body 512a extends from proximal end of conical portion 506a to distal end of another conical portion 510a, where the two portions 506a and 510a are connected by the elongated cylindrical balloon body portion 512a of a length in a range of 40±0.127 mm and a diameter in a range of 5.842±0.508 mm. Thickness of wall of balloon body portion 512a is approximately 0.0254 mm. A proximal end of conical portion 510a further connects to a proximal neck 514a having an inner diameter of 1.524 mm. Necks 504a and 514a each have a length of approximately 5.004 mm. A distal portion 516a of a catheter shaft extends distally from distal neck 514a. An average burst pressure of balloon 500a is 22 ATM, which is made using PET in an embodiment.

FIG. 5B illustrates a side view of an exemplary embodiment of an outer balloon 500b of a device for treating in-stent restenosis and vascular stenosis, in accordance with some embodiments of the present specification. A distal neck 504b of the outer balloon 500b has an internal diameter in a range of 1.879±0.127 mm. Distal neck 504b connects at its proximal end to a distal conical portion 506b of outer balloon 500b. A balloon body 512b extends from proximal end of conical portion 506b to distal end of another conical portion 510b, where the two portions 506b and 510b are connected by elongated cylindrical balloon body portion 512b of a length in a range of 43.99±2.006 mm and a diameter in a range of 5.994±0.9906 mm. A proximal end of conical portion 510b further connects to a proximal neck 514b having an inner diameter of 1.879 mm. Necks 504b and 514b each have a length of approximately 5.004 mm. A nominal pressure of outer balloon 500b is 7 ATM, and a minimum burst pressure of outer balloon 500b is 20 ATM, which is made using nylon in an embodiment.

FIG. 5C illustrates a side view of another exemplary embodiment of inner balloon 500c of a device for treating in-stent restenosis and vascular stenosis, in accordance with some embodiments of the present specification. A distal neck 504c of the inner balloon 500c has an internal diameter in a range of 1.1938±0.127 mm. Distal neck 504c connects at its distal end to a distal conical portion 506c of a balloon 500c. A balloon body 512c extends from proximal end of conical portion 506c to distal end of another conical portion 510c, where the two portions 506c and 510c are connected by elongated cylindrical balloon body portion 512c of a length in a range of 40.00±0.990 mm and a diameter in a range of 8.00±0.305 mm. A proximal end of conical portion 510c further connects to a proximal neck 514c having an inner diameter of 1.600 mm. Necks 504c and 514c each have a length of approximately 5.004 mm. A nominal pressure of inner balloon 500c is 2 ATM, and a minimum burst pressure of inner balloon 500c is 15 ATM, which is made using nylon in an embodiment.

Preferably, catheters of the present specification are designed for dual-mode operation that enables the concurrent delivery of thermal ablative energy as well as acoustic pressure waves. The following section discusses exemplary embodiments of a double balloon catheter design for use by some embodiments of the present specification. An exemplary double balloon catheter is disclosed in U.S. Pat. No. 11,331,140, which is incorporated herein by reference in its entirety.

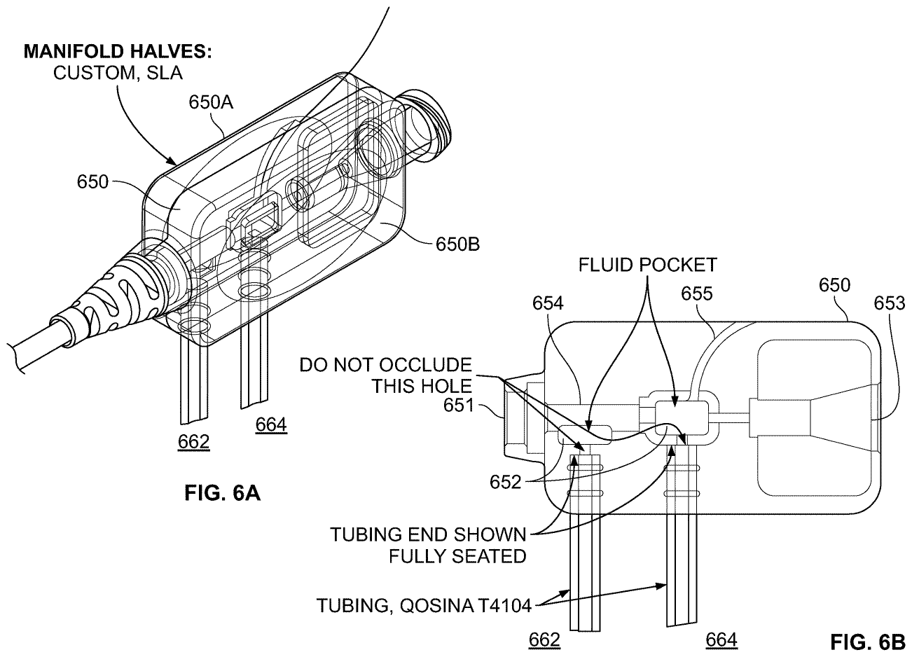

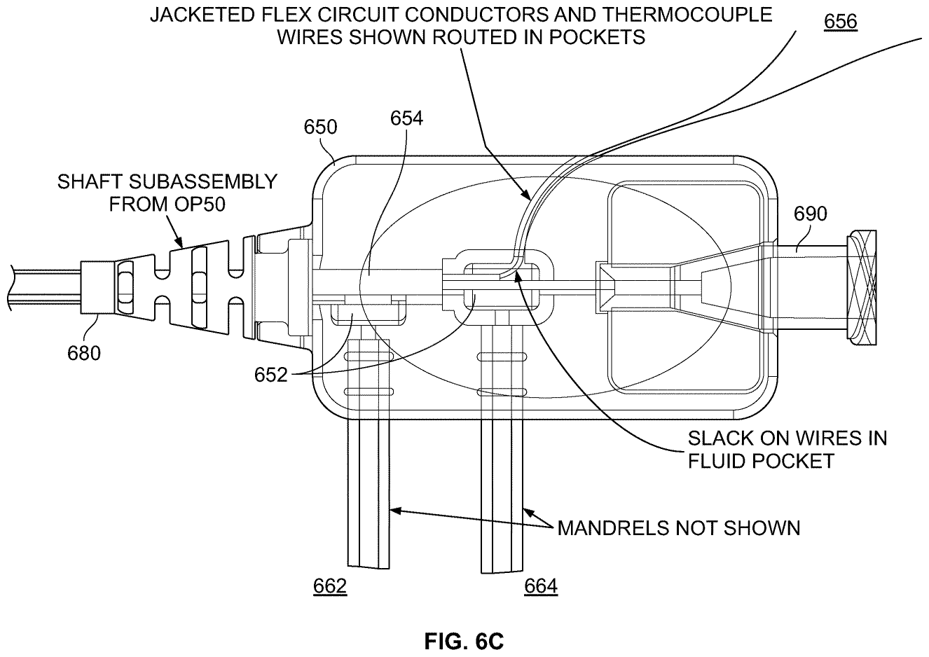

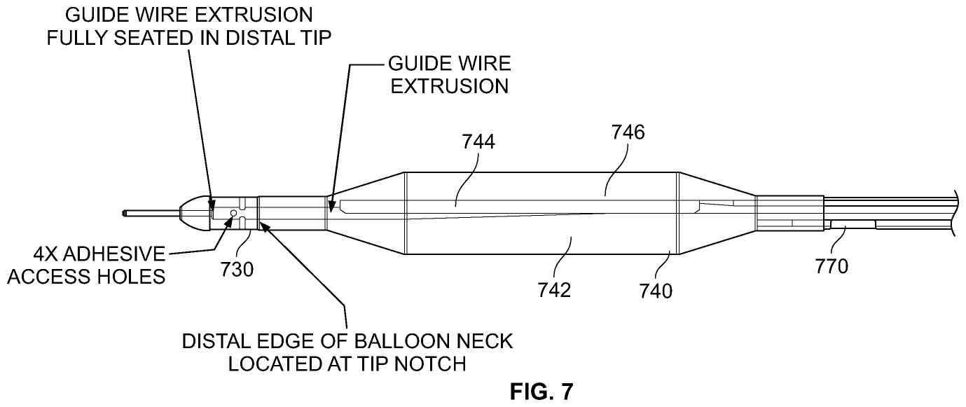

FIGS. 6A-C, 7, 8, and 9 disclose a catheter 900, or portions thereof, having a proximal end comprising a manifold 650, 950, a distal end 740, 940 comprising an atraumatic tip 830, 730, 930, heating electrode 746, 946, inner balloon 744, 944 and outer balloon 742, 942 and a longitudinal shaft 770, 970 extending between the proximal and distal ends and comprising a plurality of lumens. The manifold 650, 950 comprises a housing enclosing or partially enclosing a connection port 690, 990, conductive wires 656, 956 (extending through a dedicated channel 655 in the housing), fluid chambers 652, 952 each positioned at the intersection between channels 662, 962, 664, 964 supplying fluid to the outer balloon 742, 942 and supplying liquid to the inner balloon 744, 944 and channel 654, 954 which comprises separate lumens that extend through the shaft 770, 970 and to each of the inner balloon 744, 944 and outer balloon 742, 942 separately. Each of the fluid chambers 652, 952 is only connected to one of the separate lumens that extend through the shaft 770, 970 and to each of the inner balloon 744, 944 and outer balloon 742, 942 separately. Preferably, the manifold housing is made of two halves 650A, 650B and comprises a first connection port 651 for receiving and coupling to shaft 770, 970 and a receiving structure 653 for holding connection port 690, 990. Coupled to the manifold 650, 950 is a strain relief 680, 980 comprising a plastic tapered structure having conformable slits positioned throughout.

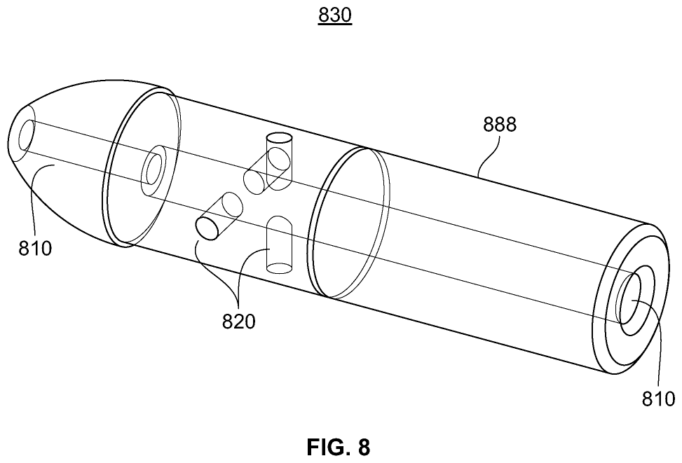

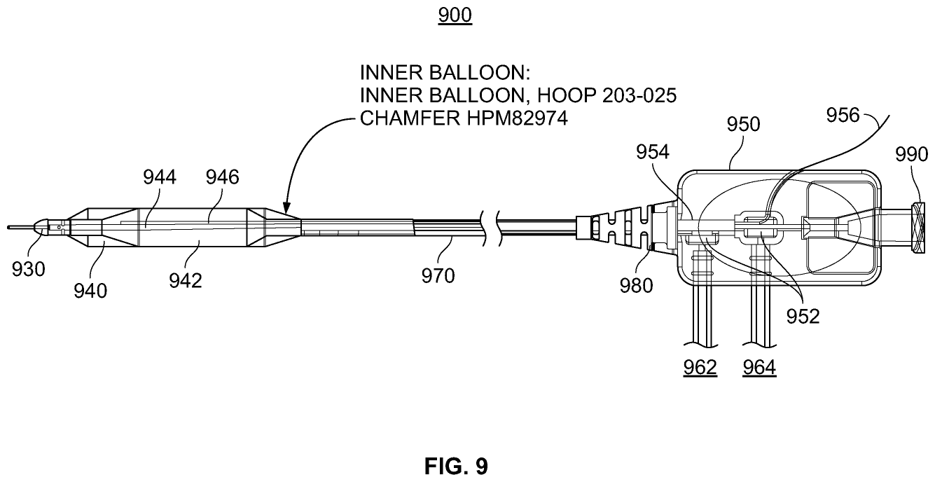

Referring to FIG. 8, the atraumatic tip 830 comprises a central lumen 810 positioned within a cylindrical housing 888 and extending to a dome shaped tip 810. Access ports 820 extend through the housing 888 and to the lumen 810.

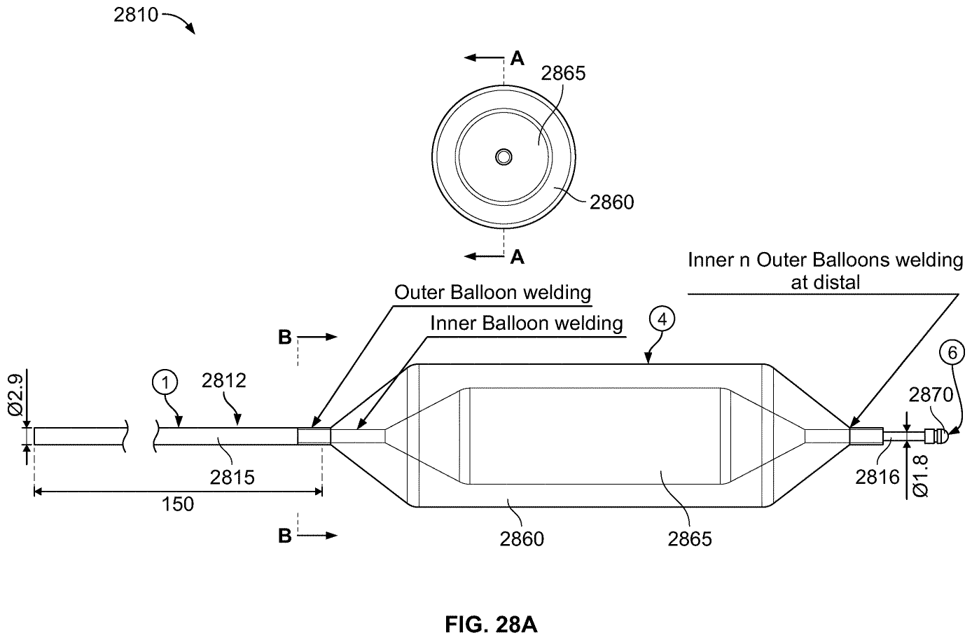

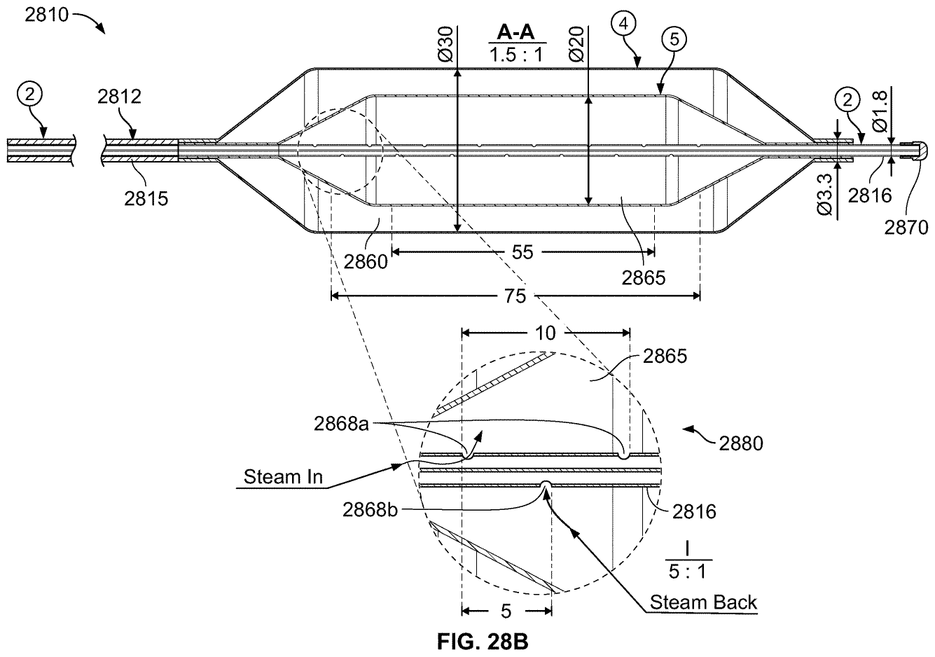

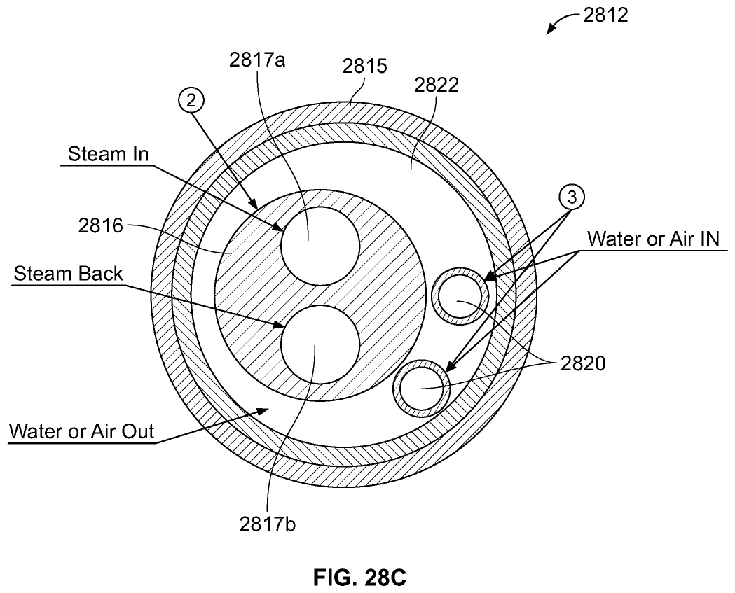

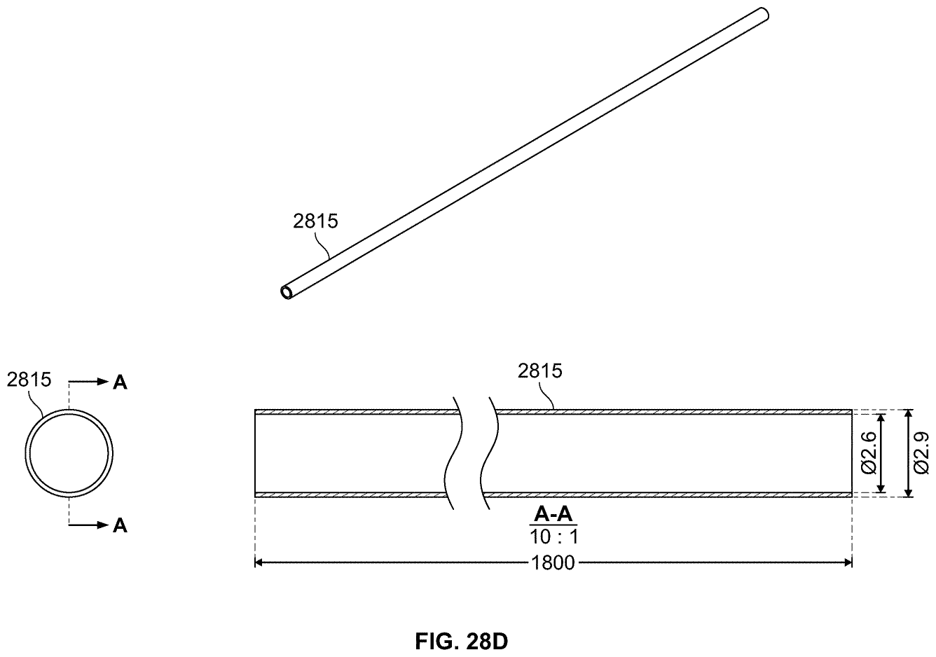

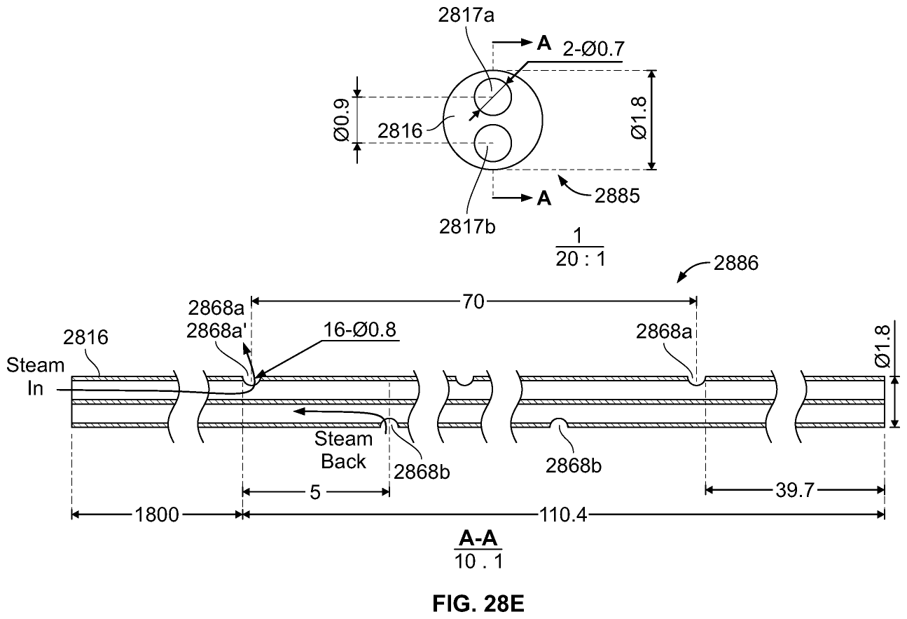

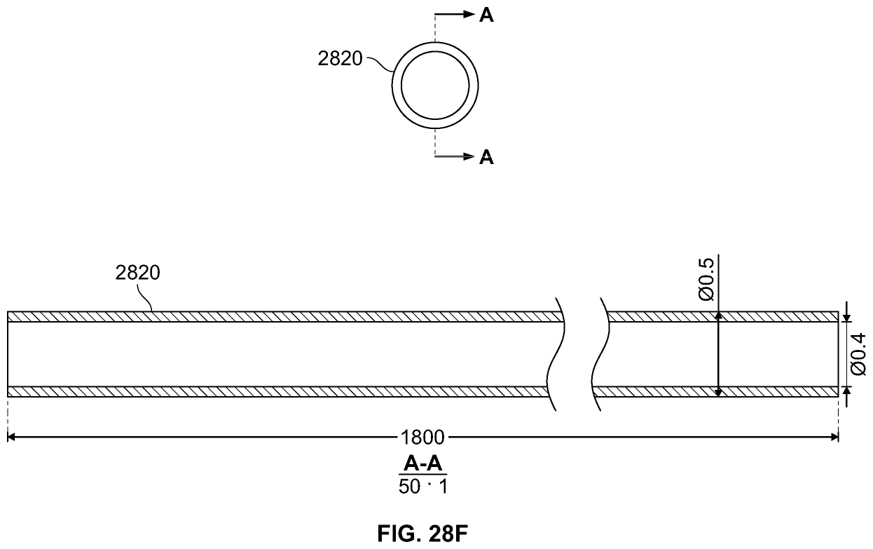

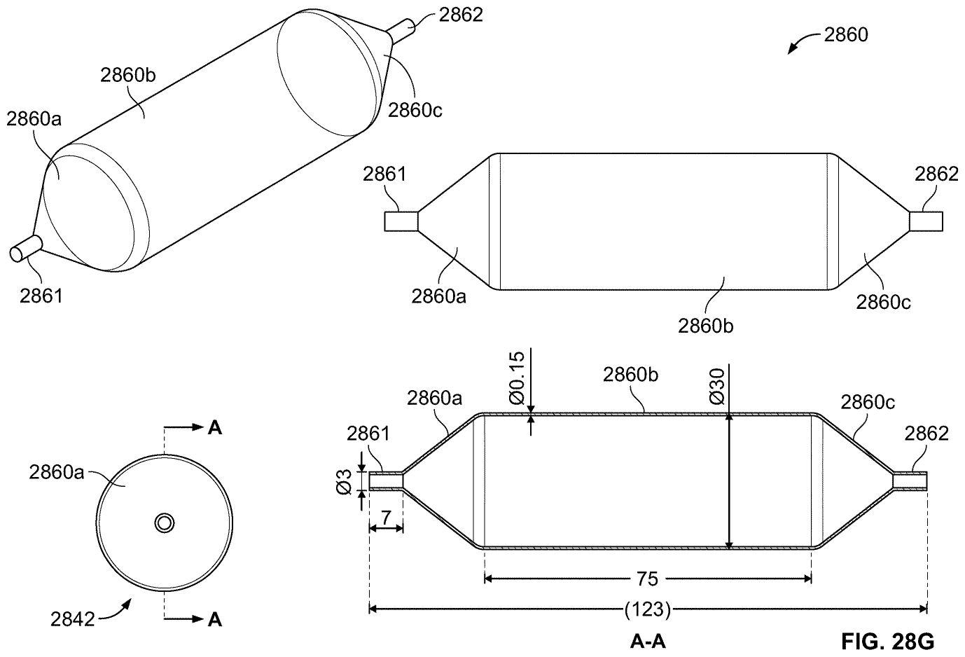

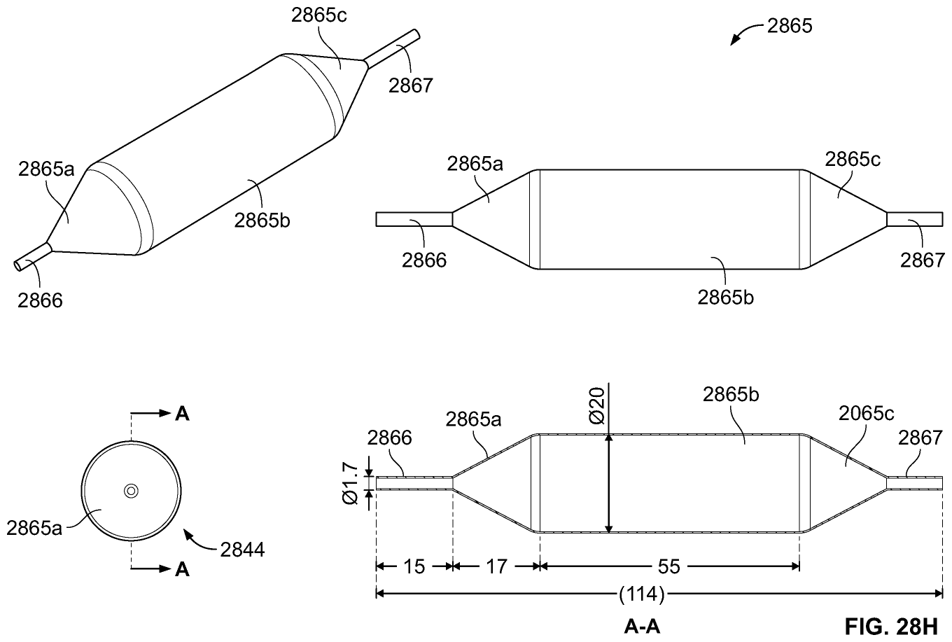

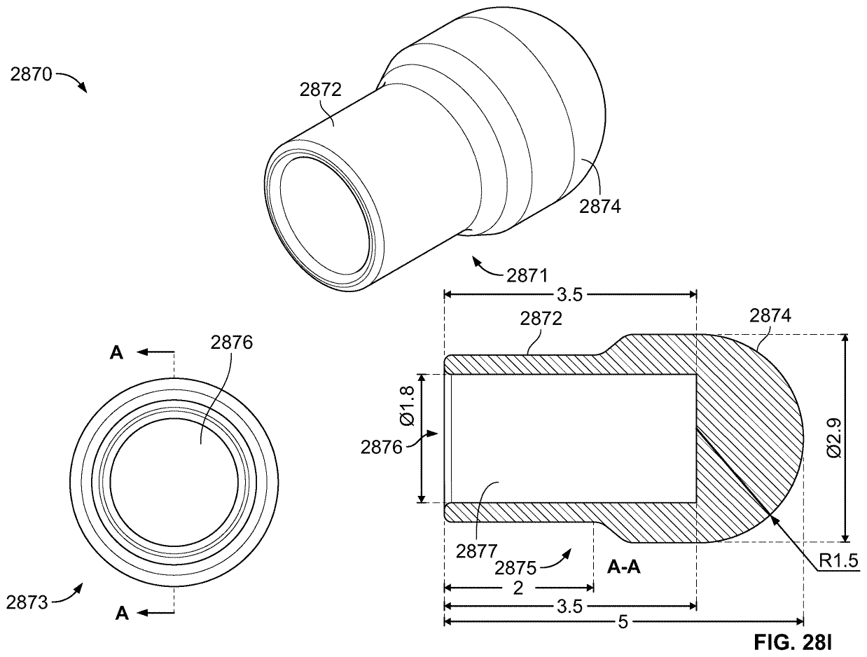

FIG. 28A illustrates longitudinal and transverse perspective views of another dual balloon catheter 2810, FIG. 28B illustrates a longitudinal cross-sectional view along with an enlarged view of a portion of the catheter 2810 and FIG. 28C illustrates a transverse cross-sectional view of an elongate body 2812 of the catheter, in accordance with some embodiments of the present specification. Referring to FIGS. 28A, 28B and 28C simultaneously, unless otherwise mentioned specifically, the elongate body 2812 has a proximal end, a distal end and a plurality of lumens.

In one embodiment, the elongate body 2812 includes an outer catheter 2815 having a lumen and an inner catheter 2816 having a first liquid infusion lumen 2817a and a second liquid suction lumen 2817b. In some embodiments, the first and second liquid lumens 2817a, 2817b are diametrically opposite to each other. The inner catheter 2816 is positioned within the lumen of the outer catheter 2815 so as to form a fluid suction pathway 2822 between the two catheters 2815, 2816. The fluid suction pathway 2822 accommodates at least one fluid infusion tube 2820.

The at least one fluid infusion tube 2820 and the fluid suction pathway 2822 are in fluid communication with an inflatable outer balloon 2860 attached to the distal end of the catheter 2810. In embodiments, distal ends of the outer catheter 2815 and of the fluid infusion tube 2820 lie or terminate at a proximal end of the outer balloon 2860 whereas a distal end of the inner catheter 2816 projects beyond a distal end of the outer balloon 2860. In some other embodiments, the distal end of the inner catheter 2816 terminates at a distal end of an expandable inner balloon 2865. During operation, a first fluid comprising 50/50 saline-contrast mix enters the fluid infusion tube 2820 from the proximal end of the catheter 2810 and exits through the suction pathway 2822 from the proximal end of the catheter 2810 after circulating through the outer balloon 2860. The first fluid is circulated through the catheter 2810 and the outer balloon 2860 using a first fluid pump which is in data communication with, and controlled by, a controller.

The first liquid infusion lumen 2817a is in fluid communication, via a plurality of liquid infusion ports 2868a, with the inflatable inner balloon 2865 attached to the distal end of the catheter 2810 and positioned within the outer balloon 2860. The second liquid suction lumen 2817b is also in fluid communication with the inflatable inner balloon 2865 via a plurality of liquid suction ports 2868b. In some embodiments, suction of the liquid suction ports 2868b is controlled by a pressure sensor controlled external pump or by a self-opening pressure valve. In some embodiments, the plurality of liquid infusion and suction ports 2868a, 2868b are located along a portion of the inner catheter 2816 lying within the inner balloon 2865.

As shown in transverse cross-sectional, longitudinal cross-sectional and perspective views of FIG. 28D, in some embodiments, the outer catheter 2815 has a length of 1800 mm, an inner diameter of 2.6 mm and an outer diameter of 2.9 mm. In some embodiments, as shown in a transverse cross-sectional view 2885 of FIG. 28E, the inner catheter 2816 has an outer diameter of 1.8 mm, the centers of the first and second liquid lumens 2817a, 2817b are separated by a distance of 0.9 mm and the internal diameter of each of the first and second liquid lumens 2817a, 2817b is 0.7 mm. In some embodiments, as shown in the longitudinal cross-sectional view 2886 of FIG. 28E, the inner catheter 2816 has a length of 1910.4 mm.

In some embodiments, seven liquid infusion ports 2868a are located on the inner catheter 2816 along the first liquid infusion lumen 2817a wherein each port 2868a has a diameter of 0.8 mm. In some embodiments, a proximal port 2868a′ is located at a distance of 1800 mm from a proximal end of the inner catheter 2816 and at a distance of 110.4 mm from a distal end of the inner catheter 2816. In some embodiments, as also shown in an enlarged view 2880 of FIG. 28B, the plurality of liquid infusion ports 2868a are spaced by a distance of 10 mm from each other. In some embodiments, six liquid suction ports 2868b are located on the inner catheter 2816 along the second liquid suction lumen 2817b wherein each port 2868b has a diameter of 0.8 mm. In some embodiments, as also shown in an enlarged view 2880 of FIG. 28B, the plurality of liquid infusion ports 2868a are spaced by a distance of 10 mm from each other and the plurality of liquid suction ports 2868b are also spaced by a distance of 10 mm from each other. Also, in some embodiments, each of the liquid suction ports 2868b is located at a distance of 5 mm from an immediately preceding and an immediately successive liquid infusion port 2868a.

As shown in transverse and longitudinal cross-sectional views of FIG. 28F, in some embodiments, fluid infusion tube 2820 has a length of 1800 mm, an inner diameter of 0.4 mm and an outer diameter of 0.5 mm.

In some embodiments, at least one flexible heating chamber, comprising a plurality of electrodes, is positioned in-line within the first liquid infusion lumen 2817a. Electrical leads connect the electrodes to a plug in a handle of the catheter. In some embodiments, the at least one flexible heating chamber is positioned in-line within the first liquid infusion lumen 2817a such that the plurality of electrodes are at least partially inside the inner balloon 2865. During operation, a liquid pump which is also in data communication with, and controlled by, the controller pumps water from a sterile water reservoir through the liquid lumen 2817a to enter a proximal end of the at least one flexible heating chamber. The at least one flexible heating chamber converts liquid into heated liquid that exits through the plurality of liquid infusion ports 2868a to inflate the inner balloon 2865 and contact the outer balloon 2860 proximate a target site with calcified plaque deposits.

The lumen 2817a extends from the inner balloon 2865 to a liquid pump which is also in data communication with, and controlled by, the controller. A proximal end of the liquid lumen 2817a, at the handle, has a luer lock connection to a sterile water/saline reservoir. Optionally, a Y-adapter is included to allow a guidewire/mapping catheter to pass through, while injecting saline into the lumen to convert to heated liquid when it passes across the electrode at the distal tip. Liquid (i.e. water/saline) is pumped from the sterile liquid reservoir via the pump through the lumen 2817a to enter a proximal end of the at least one flexible heating chamber. The at least one flexible heating chamber converts liquid into heated liquid that exits through at least one infusion port 2868a, positioned on a portion of the body 2812 lying within the inner balloon 2865, to inflate the inner balloon 2865. In some embodiments, the flexible heating chamber is attached to the inner balloon 2865. In some embodiments, the inner balloon 2865 is configured to inflate only within a predefined location within the outer balloon 2860. In some embodiments, the outer balloon 2860 includes retaining structures to ensure the inner balloon 2865 inflates within the predefined location. In some embodiments, the inner balloon 2865 is configured to withstand an operating temperature of at least 125° C. In some embodiments, the inner balloon 2865 is configured to have a burst pressure rating of at least 5 atm at the rated temperature. In some embodiments, the inner balloon 2865 is configured to withstand at least 50 cycles of 0-180 second ablation treatments conducted using at least a 30 watt power level. In some embodiments, the outer balloon 2860 is configured to withstand an operating temperature of at least 110° C.

An ablation zone or hot zone is created at the area of contact between the inner balloon 2865 and the outer balloon 2860, such that thermal energy from the inner balloon 2865 passes through the outer balloon 2860 to the target site. The elongate body 2812 and portions of the outer balloon 2860, excluding the hot zone, remain cool. For deflation, liquid is suctioned out of the inner balloon 2865 through the plurality of liquid suction ports 2868b and via the second liquid suction lumen 2817b.

Referring to side and various perspective views of FIG. 28G, in an embodiment, the outer balloon 2860 has a compound shape (when in fully expanded state) comprising of a substantially cylindrical portion 2860b having substantially tapering or conical proximal and distal ends 2860a, 2860c (shown in side perspective view 2842 of the outer balloon 2860 in FIG. 28G). The ends extend into tube-like structures 2861 and 2862, which help in keeping the catheter in place when it is passed through the balloon. In an embodiment, the outer balloon 2860 has a length of 123 mm between the tube like structures 2861 and 2862, the substantially cylindrical portion 2860b has a length of 75 mm and an outer diameter of 30 mm, each of the tube like structures 2861, 2862 has a length of 7 mm and an internal diameter of 3 mm, and each of the substantially tapering or conical ends 2860a, 2860c has a length of 7 mm. In one embodiment, the outer balloon 2860 is made up of a harder material relative to the inner balloon, such as PET, that has a thickness of 0.15 mm.

Referring to side and various perspective views of FIG. 28H, in an embodiment, the inner balloon 2865 has a compound shape (when in fully inflated state) comprising of a substantially cylindrical portion 2865b having substantially tapering or conical proximal and distal ends 2865a, 2865c (also shown in a side perspective view 2844 of the inner balloon 2865). The ends extend into tube like structures 2866 and 2867, which help in keeping the catheter in place when it is passed through the balloon. In an embodiment, the inner balloon 2865 has a length of 114 mm between the tube like structures 2866 and 2867, the substantially cylindrical portion 2865b has a length of 55 mm and a diameter of 20 mm, each of the tube like structures 2866, 2867 has a length of 15 mm and a diameter of 1.7 mm, and each of the substantially tapering or conical ends 2865a, 2865c has a length of 17 mm. In one embodiment, the inner balloon 2865 is made up of a more flexible material relative to the outer balloon, such as latex.

In some embodiments, the inner balloon 2865 is movable along a portion of the inner catheter 2816 within the outer balloon 2860 to better position the inner balloon within the outer balloon and ensure proper contact of the inner balloon with the outer balloon, using a wire mechanism in a handle at the proximal end of the catheter 2810.

In an embodiment, the distal end of the elongate body 2812 has a bulbous tip 2870 which may have an oval or olive-shaped tip (olive-tip) to make the distal end relatively atraumatic to tissues when the catheter 2810 is introduced into a body lumen. FIG. 28I illustrates a perspective view 2871, a side perspective view 2873 and a longitudinal cross-sectional view 2875 of the bulbous or olive tip 2870, in accordance with an embodiment of the present specification. The tip 2870 has a proximal substantially cylindrical portion 2872 that leads to a substantially oval or olive distal portion 2874. The cylindrical portion 2872 has an opening 2876, at a proximal end, leading to a cylindrical pathway 2877 that accommodates a portion of the distal end of the catheter 2810 (that is, the distal end of the inner catheter 2816) when the tip 2870 is mounted on the distal end of the catheter 2810.

In an embodiment, a total length of the oval or olive tip 2870 is 5 mm. In an embodiment, the proximal substantially cylindrical portion 2872 has a length of 2 mm. In an embodiment, the opening 2876 has a diameter of 1.8 mm and the cylindrical pathway 2877 has a length of 3.5 mm. In an embodiment, the substantially oval or olive distal portion 2874 has a width of 2.9 mm and a radius of 1.5 mm.

In some embodiments, an entire surface of the catheter 2810, including the balloons 2860, 2865, oval or olive tip 2870, is coated with heparin or with a biocompatible material.

Optionally, the inner balloon 2865 ‘floats’ within the outer balloon and axial force may be applied by a user through a guide sheath, further deforming the outer balloon and pushing the inner balloon 2865 in any orientation and creating an ablation zone where tissue contact is made by the outer balloon and the inner balloon intersection. Width of the ablation zone could be increased by increasing axial force transmitted via the guide sheath to the outer and the inner balloons. Additionally, the inner balloon can float relative to the outer balloon and guide sheath pressure can be used to move the inner balloon relative to the outer balloon.

Pressure is regulated in the inflated balloons using pressure valves in the outflow channel connected to the inner balloon and the outflow channel connected to the outer balloon. In embodiments, the pressure is maintained between 0 psi and 50 psi in both the outer and the inner balloons. In some embodiments, a check valve (self-opening valve) is in the outflow channel coming from the outer balloon and set at a Pound-force per Square Inch (psi) value in a range of 0 to 5 psi. In some embodiments, another check valve (self-opening valve) is in the outflow channel coming from the inner balloon and set at a psi value in a range of 0.5 to 10. In some embodiments, the outer balloon valve opens between a pressure of 0 to 1.0 psi. In some embodiments, the inner balloon pressure is released above 2.0 to 3.0 psi. The ratio of volume maintained for the inner balloon and the outer balloon is dynamic, and is controlled by controlling the pressure in the balloons. In some embodiments, the outer balloon is inflated first to a maximum volume, the inner balloon is “primed”, and then heated liquid is created. The outer balloon volume does not change as heated liquid is created, due to the pressure valve control, while the inner balloon volume increases to 100% of its volume, which, in some embodiments, equals approximately 40% of the volume of the outer balloon. The check valves serve to automatically control pressure in the balloons. In some embodiments, the pressure in the inner balloon is same as or greater than the pressure in the outer balloon. Therefore, the pressure remains within its pre-defined range, otherwise the check valve opens. In alternative embodiments, an active pressure management system, such as suction, is used to regulate the pressure.

In order to achieve optimal heat tolerance, inflation, and compliance, it is important to use an appropriate material for manufacturing the outer and inner balloons. If the softening temperature (Tg) of a balloon's material is too low, the balloon may deform during use when exposed to heat. For example, the Tg of PET is 75° C. This means that after just one use, the PET balloon may deform and may not be useable for conducting additional ablation shots of a given PV or of other PVs in the patient. Therefore, it is desirable to use a material that has a Tg greater than 100° C. to be functional. In embodiments, there are two balloons, where each balloon has a different Tg value. In embodiments, the Tg value of each balloon is within a range of 60° C. to about 200° C. In some embodiments, the Tg is 80° C. In some embodiments, the Tg is 150° C.

It is also desirable to use a material that has a sufficiently wide elasticity range at various operating temperatures. If the elasticity range is too low, the yield point is passed during operation and the balloon deforms such that the ablation zone may not be properly positioned during operation. In embodiments, thermo-plastic copolyester material such as those provided by Arnitel, may be used, which has a Tg of about 150° C.

In embodiments, material of the inner balloon is semi-compliant, implying that the material eliminates any folds that may have been present during packaging, and conforms to the vessel anatomy for better contact. A compliant balloon is likely to have a fixed volume at a fixed pressure. It is desirable that material of the inner balloon is more rigid than that of the outer balloon, so as to maintain a certain shape and is not easily deformed by pressing against cardiac tissue. Some of the semi-compliant balloon materials, for example PEBA families, face mechanical and thermal challenges when introduced to heat. Therefore, one possible balloon material is a copolymer called Arnitel. Arnitel is also relatively semi-compliant but has higher softening and melt temperatures versus standard PEBA polymers. Materials such as Arnitel may be used to make the inner balloon and shaft applications, in accordance with the embodiments of the present specification. An advantage of using it as a shaft material is that it is thermally bondable with inner balloons currently made using PET, thereby eliminating the need to use an adhesive bonding process.

In embodiments, inner and outer balloons are made using compliance-adjustable materials. In one embodiment, shape memory material is used which maintain a ratio of 1.1:1 (outer balloon:inner balloon) across diameters ranging from 2 to 50 mm. In different embodiments, the outer balloon diameter is 1.1 to 1.5 times the diameter of the inner balloon when maximally inflated. Furthermore, the inner balloon diameter may range from 2 mm to 50 mm, while outer balloon diameter may range correspondingly from 2.2 mm to 75 mm. The inner balloon matches the native vessel or the stent diameter, and the outer balloon is between 5% and 50% larger in diameter than the inner balloon. The length of the inner and outer balloons can range from 5 mm to 80 mm.

In some embodiments, the heated liquid delivered to the inner balloon is selected to provide one or more desired properties including electrical conductivity for resistive heating, predictable heat capacity, reduced gas formation, reduced ionic precipitation, controlled osmolality, controlled pH, or compatibility with one or more temperature and pressure sensors.

In various embodiments, the catheter is configured such that the heated liquid delivered to (or generated within) an ablation balloon and/or an intermediate chamber is present as (i) a heated liquid or a two-phase mixture comprising a liquid phase and a vapor phase. The two-phase mixture may be formed intentionally (e.g., partial vaporization) or as a consequence of pressure/temperature control, and may be configured to improve heat transfer, shorten time-to-target temperature, increase uniformity of balloon temperature, or improve controllability of delivered thermal dose. Therefore, in some embodiments, the heated fluid comprises heated saline solution in a liquid phase that is circulated through a lumen and/or chamber to deliver thermal energy through the balloon wall. In still other embodiments, the heated fluid comprises a two-phase working fluid, where vapor and liquid coexist within the balloon and/or chamber, thereby enabling latent-heat-driven energy transfer during condensation and/or boiling.

In some embodiments, the catheter comprises a heating chamber positioned within the inner balloon (or the inner balloon itself serves as the heating volume). In certain embodiments, the heating chamber includes a phase-separation interface configured to preferentially pass liquid while substantially restricting vapor flow, thereby reducing or preventing introduction of vapor liquid into the balloon inflation volume when liquid delivery is desired. Liquid retained within the chamber may be recirculated, replenished, and/or removed through one or more lumens.

In other embodiments, the phase-separation interface is omitted and the heating element directly heats the liquid within the inner balloon so that the heated liquid delivered to the balloon comprises hot liquid or two-phase fluid depending on commanded operating conditions.

In various embodiments, the heating element assembly is configured to improve manufacturability and safety by reducing the likelihood of localized overheating, electrical arcing, and uncontrolled thermal transients. In some embodiments, the heating element (e.g., one or more electrodes, resistive traces, or other heater structures) includes a dielectric stand-off, encapsulation layer, or insulating overmold that increases electrical isolation and reduces electric-field concentration at corners, edges, or other geometric discontinuities. Such dielectric structures may reduce the risk of direct arcing and may reduce localized film boiling or hotspot formation that can occur near sharp edges under high heat flux conditions.

In some embodiments, the catheter includes redundant temperature sensing configured to provide both heater-proximal and tissue-proximal thermal feedback. For example, a first temperature sensor may be positioned adjacent to or integrated with the heating element to measure a heater-adjacent temperature and detect rapid temperature rise. A second temperature sensor may be positioned at or near an interface region, such as within the inner balloon, within or on the outer balloon wall, or on a shaft region adjacent the balloon segment, to measure a temperature more representative of the tissue-interface condition. The controller may use both measurements to control heater power and to implement safety interlocks, such as limiting maximum heater-adjacent temperature and limiting maximum interface temperature.

In various embodiments, the system includes power-limiting and/or self-limiting heating features. In some embodiments, the heating element comprises a positive temperature coefficient (PTC) heater configured to reduce power output as temperature increases. In some embodiments, the controller implements current limiting, voltage limiting, and/or a duty-cycle cap to limit maximum delivered power. In some embodiments, the system includes dry-out detection configured to identify reduced or absent fluid contact with the heating element, for example by detecting a change in heater impedance, resistance, current draw, or thermal response that is indicative of insufficient fluid presence. Upon detecting dry-out or another fault condition, the controller may reduce power, discontinue heating, and/or initiate a deflation sequence to mitigate risk.

In various embodiments, the catheter comprises an outer balloon positioned radially outward of an inner balloon and configured to contact (directly or indirectly) an inner surface of a body lumen (e.g., a vessel wall). The outer balloon may be inflated with an outer balloon fill fluid (also referred to herein as a “cooling fluid,” “insulating fluid,” “barrier fluid,” or “coupling fluid”) that is selected to provide one or more functions including (i) thermal insulation between a heated inner balloon and blood or non-target tissue, (ii) heat extraction and cooling of the balloon wall and/or tissue interface, (iii) modulation of heat flux to define treatment zones, and/or (iv) in certain embodiments, acoustic coupling for intravascular lithotripsy (IVL) energy delivery.

Accordingly, references herein to “cooling fluid,” “insulating fluid,” or “outer balloon fill fluid” are exemplary only, and in various embodiments the outer balloon is inflatable with a gas, liquid, vapor, or multi-phase fluid, optionally circulated and/or temperature-controlled, to provide thermal insulation, active cooling, heat-flux shaping, and/or acoustic coupling depending on the particular treatment mode and commanded operating conditions.

As used herein, the term “outer balloon fill fluid” includes liquids, gases, vapors, and mixtures thereof, including single-phase fluids and multi-phase fluids. In some embodiments, the outer balloon fill fluid comprises a gas such as air, nitrogen, carbon dioxide, nitrous oxide, helium, argon, or other biocompatible gas. Gas-filled embodiments may provide relatively higher thermal resistance and may reduce thermal transfer away from the heated inner balloon, thereby enhancing insulation of blood and/or limiting collateral heating.

In other embodiments, the outer balloon fill fluid comprises a liquid such as sterile water, saline, dextrose solution, buffered solutions, balanced electrolyte solutions (e.g., lactated Ringer's or Plasma-Lyte®), radiopaque contrast media, or mixtures thereof. Liquid-filled embodiments may provide increased thermal conductivity relative to gas-filled embodiments and may be used to promote more uniform temperature distribution along the outer balloon wall and/or to provide improved thermal coupling and/or acoustic coupling. In some embodiments, the outer balloon fill fluid includes a contrast agent and/or other additives (e.g., surfactants, anti-foaming agents, dyes) provided the resulting fluid remains biocompatible for intravascular use.

In various embodiments, the outer balloon fill fluid is delivered at a temperature selected to produce a desired thermal effect. For example, the outer balloon fill fluid may be delivered as a cooling fluid (e.g., at or below body temperature) and circulated through the outer balloon to remove heat from the balloon wall, to limit peak interface temperature, to protect blood or adjacent tissue, and/or to establish a thermal boundary condition that shapes the radial and axial temperature profile during treatment. In other embodiments, the outer balloon fill fluid may be delivered at an elevated temperature (e.g., warmed fluid) to reduce thermal gradients, improve uniformity, or reduce thermal shock, while still maintaining a commanded maximum tissue-interface temperature.

In some embodiments, the outer balloon fill fluid is static during a treatment interval, and in other embodiments the outer balloon fill fluid is flowed or circulated through the outer balloon via one or more lumens to provide active cooling, active insulation control, degassing, and/or pressure stabilization. In certain embodiments, the controller adjusts one or more parameters including flow rate, pressure, volume, temperature, and/or composition of the outer balloon fill fluid to control thermal impedance between the inner balloon and the target tissue and/or to control the extent of thermal insulation of blood.

In some embodiments, the outer balloon fill fluid is selected and/or controlled to establish a variable thermal barrier. For example, the controller may command the outer balloon to operate in a first state in which the outer balloon fill fluid (e.g., a gas or low-conductivity medium) provides increased insulation and reduced heat flux, and a second state in which the outer balloon fill fluid (e.g., a liquid or higher-conductivity medium) provides increased thermal coupling and/or increased heat extraction. In some embodiments, the catheter is configured so that the outer balloon fill fluid and the relative inflation state of the inner and outer balloons cooperatively define regions of increased thermal transfer (“ablation zones”) and regions of reduced thermal transfer (“non-ablation zones”) along the outer balloon wall.

In various embodiments, thermal energy is transferred from a heated working fluid to target tissue through a controllable thermal resistance stack that may be modeled as a series thermal circuit. By way of example, heat transfer may occur along the pathway: heated fluid→inner balloon wall→(contact region or gap region)→outer balloon wall→tissue.

In another embodiments, thermal energy is transferred from a cold (cryogen) working fluid to target tissue through a controllable thermal resistance stack that may be modeled as a series thermal circuit. By way of example, ablative energy transfer may occur along the pathway: cold fluid→inner balloon wall→(contact region or gap region)→outer balloon wall→tissue.

In a contact state, the inner balloon and the outer balloon are in contact over one or more regions such that an intervening gap thickness is approximately zero (or sufficiently small to be negligible). In such contact regions, the effective thermal resistance between the heated fluid and the tissue is reduced, resulting in a relatively higher heat flux through the outer balloon wall to the tissue (e.g., defining one or more ablation zones).

In a non-contact state, a gap is present between at least a portion of the inner balloon and the outer balloon. The gap may be filled with a gas, a liquid, a vapor, or a multi-phase medium. In such non-contact regions, the presence of the gap increases the effective thermal resistance between the heated fluid and the tissue, resulting in a relatively lower heat flux through the outer balloon wall (e.g., defining one or more non-ablation or insulation zones).

In some embodiments, the gap thickness is controlled to shape a spatial and/or temporal thermal delivery profile. For example, the gap thickness in the non-contact state may be within a range of about 0.05 mm to about 2.0 mm, including any sub-range therein. The gap thickness and/or the extent of contact area may be established or adjusted by one or more of: (i) a commanded pressure differential between the outer balloon and the inner balloon (e.g., Pouter-Pinner); (ii) a compliance mismatch between the balloons (e.g., an outer semi-compliant balloon and an inner non-compliant balloon); (iii) one or more mechanical spacers, ribs, stand-offs, or surface features configured to maintain a minimum separation distance in selected regions; and/or (iv) material behavior such as a shape-memory ratio or other geometry-dependent compliance behavior that changes balloon conformation as a function of pressure, temperature, or time.

In embodiments configured for thermal therapy, the inner balloon may be configured to contain and/or circulate a heated working fluid and the outer balloon may be positioned between the inner balloon and the vessel wall. The controller may adjust the inner and outer balloon inflation states to control whether and where the inner balloon is in contact with the outer balloon. In some embodiments, a contact state is established in which portions of the inner balloon contact portions of the outer balloon, thereby reducing thermal resistance and increasing heat flux to tissue through those portions of the outer balloon. In some embodiments, an insulation state is established in which a gap (e.g., a gas gap, low-conductivity medium, or reduced-contact region) is maintained between at least a portion of the inner balloon and the outer balloon, thereby increasing thermal resistance and reducing heat flux through those portions of the outer balloon. In this manner, the controller can modulate thermal contact versus thermal insulation by commanding pressure and/or volume changes in one or both balloons, thereby shaping a spatial and/or temporal profile of delivered thermal energy.

In various embodiments, temperatures within the catheter may differ among a heating chamber (if present), a fluid volume within the inner balloon, and one or more balloon walls. For example, where a heating chamber is used to heat a working fluid, the temperature of the heating chamber and/or the heating element may be higher than a temperature of the inner balloon wall during operation. The inner balloon wall temperature may be lower than the chamber temperature due to one or more of fluid mixing, fluid flow or circulation through the inner balloon, thermal losses along the fluid path, and/or heater duty cycling that produces a time-varying thermal input. In addition, the outer balloon wall temperature may be actively limited during therapy. For example, the outer balloon wall temperature may be limited by selection and/or control of the outer balloon medium (e.g., gas versus liquid versus mixture), by control of a contact duty cycle between the inner balloon and the outer balloon (thereby modulating thermal resistance and heat flux), and/or by optional cooling of the outer balloon medium using a circulation path, cooling loop, or heat exchanger coupled to the outer balloon.

In various embodiments, one or both balloons may comprise multi-layer wall constructions to support thermal performance, mechanical strength, and biocompatibility. In some embodiments, the inner balloon comprises a multi-layer structure including a thin inner liner configured to provide a fluid-impermeable barrier, a structural layer configured to provide hoop strength and compliance characteristics, and an optional conductive layer or patterned conductive traces configured to support resistive heating and/or sensing. In some embodiments, the outer balloon comprises a structural layer configured to provide vessel apposition and mechanical performance, and further includes optional thermal barrier bands or regions configured to increase thermal resistance in selected areas (e.g., proximal and distal edge regions) and reduce unwanted heating. In some embodiments, the outer balloon further includes one or more surface coatings, such as a lubricious coating to reduce insertion friction and/or an anticoagulant coating (e.g., heparin or heparin-containing coating) to reduce thrombus formation, as well as other coatings or surface treatments suitable for intravascular use.

In one non-limiting example, the system is operated in a contact state and a non-contact state to demonstrate contact-modulated thermal transfer. The inner balloon is inflated to an operating pressure within a range of about 2 atm to about 10 atm. The outer balloon inflation pressure may be selected based on operating mode. For example, in a thermal balloon angioplasty-only mode, the outer balloon may be inflated to approximately 1 atm in some embodiments, while in a combined thermal and lithotripsy mode the outer balloon may be inflated within a similar range as the inner balloon (e.g., about 2 atm to about 10 atm), such that the balloons are deployed for vessel apposition and energy delivery.

In a contact state defining an ablation zone, the inner balloon contacts the inner surface of the outer balloon such that the separation distance is approximately zero (i.e., no measurable gap), thereby reducing thermal resistance and increasing heat flux to tissue through the outer balloon wall. In a non-contact state defining a non-ablation or insulation zone, a separation gap is present between the inner balloon and outer balloon, for example about 1 mm to about 2 mm, thereby increasing thermal resistance and reducing heat flux through the corresponding region of the outer balloon. In some embodiments, the contact state and non-contact state are produced by setting a slight pressure differential such that the outer balloon pressure is slightly higher than the inner balloon pressure, thereby forming a controlled thin layer of fluid between the balloons in regions intended to be insulated. Temperature profiles measured at the inner balloon fluid, the outer balloon fluid, and at a tissue-adjacent depth (e.g., 1 mm) demonstrate a higher temperature rise rate and/or higher peak temperature in contact regions relative to non-contact regions under otherwise similar heating conditions, illustrating controllable formation of ablation zones and non-ablation zones.

In various embodiments, the catheter includes thermal edge protection features configured to reduce injury at proximal and distal edges of the treatment region. In some embodiments, the outer balloon comprises increased wall thickness at one or both ends of the treatment region to increase thermal resistance and reduce heat flux at edges. In some embodiments, the outer balloon includes one or more polymer bands, sleeves, or regions formed of a lower thermal conductivity material disposed at proximal and/or distal portions of the outer balloon to provide reduced heat transfer at edges. In some embodiments, edge protection features are combined with control strategies that maintain cooler proximal and distal zones relative to a central treatment zone, thereby reducing the likelihood of edge-related thermal injury while delivering a prescribed thermal dose to a target region.

In various embodiments, contact between the inner balloon and the outer balloon is established and controlled based on commanded pressure and geometry. In addition, or alternatively, the catheter may include one or more sensors configured to directly detect a contact state and/or estimate a separation distance (gap thickness) between the inner balloon and the outer balloon. Such sensing may improve repeatability of therapy delivery, confirm formation of intended ablation zones, and support closed-loop control of contact-modulated heat transfer.

In various embodiments, the catheter is configured to improve thermal uniformity and reduce localized overheating by using multi-point temperature sensing, controlled modulation of a contact state between balloons, and structural features configured to reduce edge-related injury.

In some embodiments, the catheter includes a plurality of temperature sensors positioned to measure temperature at multiple locations along the treatment region. For example, two, three, four, or more temperature sensors may be spaced axially along the balloon length and/or distributed circumferentially around the balloon to detect spatial temperature non-uniformity. Temperature sensors may be located within the inner balloon volume, within or on the outer balloon wall, on or within the catheter shaft adjacent the balloon segment, or combinations thereof. In other embodiments, the catheter includes a fiber optic temperature sensing system, such as distributed temperature sensing (DTS), configured to measure a temperature profile along a length of the shaft and/or along at least a portion of the balloon segment. The controller may use such multi-sensor measurements to estimate an interface temperature distribution, detect localized hotspots, and adjust therapy parameters to improve uniformity.

In some embodiments, the catheter includes capacitive sensing configured to detect contact or proximity across the inner and outer balloon walls. For example, one or more electrodes or conductive traces may be positioned on or within one or both balloon walls and used to measure a capacitance that varies as a function of separation distance. In some embodiments, the catheter includes an ultrasound-based sensor configured to measure a gap thickness using pulse-echo techniques, for example by transmitting one or more ultrasound pulses and measuring a time-of-flight or reflection signature indicative of balloon-to-balloon separation. In some embodiments, the catheter includes impedance-based sensing configured to detect contact or proximity, for example using patterned conductive layers and measuring an impedance that varies with contact area and/or intervening medium. In some embodiments, the catheter includes optical sensing configured to detect contact or proximity, for example by measuring reflected light intensity or spectral changes that vary with gap thickness and/or contact area.

In various embodiments, the controller uses the measured contact state and/or measured gap thickness to (i) confirm that a commanded treatment geometry has been achieved, including formation of one or more ablation zones and non-ablation zones, (ii) adjust one or more inflation parameters in real time, such as a pressure differential between the outer balloon and the inner balloon, to maintain a desired contact pattern and corresponding heat flux distribution, and/or (iii) record a contact duty cycle and/or a contact map as part of a treatment record. Such contact sensing and control may be particularly beneficial in embodiments configured to create selective or patterned ablation (for example, using inner balloon extensions that produce discrete contact regions), where verification and maintenance of the intended contact pattern can improve consistency and safety of therapy delivery.

A double balloon configuration is incorporated towards a distal end of the catheter which is useful in minimizing ischemic risk while maintaining therapeutic effectiveness. The catheter is manufactured using a material such as Pebax polymer or any known material for endovascular catheter construction. The material is combined with advanced biocompatible coatings that enhance durability, flexibility, and thermal efficiency, of the catheter device, while maintaining compatibility with existing guidewire and catheter systems. Optionally, the catheter includes imbedded Platinum-iridium electrodes. Diameter of the catheter is in a range from 1 millimeter (mm) to 5 mm. A modular design of the catheter allows adaptation for different vessel sizes and anatomical challenges, with interchangeable balloon components for diverse applications. Additionally, the catheter is compatible with 6F or larger guiding catheters.

A distal end of the catheter is configured with a dual-balloon structure that is used as a positioning element within a vascular lumen to position the distal end proximate a target site. The dual-balloon structure is coaxially designed with an inner balloon surrounded by an outer balloon. In embodiments, shape-memory balloons are used which maintain 1.1:1 ratio (outer balloon:inner balloon) across diameters of 2-50 mm without manual sizing. The inner and outer balloons are cylindrical in shape, in some embodiments. Size of the balloon can be customized for various anatomical applications such as and not limited to applications in iliac, femoropopliteal, and below-knee arteries. The different configurations can meet varying energy requirements for different plaque morphologies.

The inner balloon is manufactured using a non-compliant PET or a comparable material, and the outer balloon is manufactured using a semi-compliant Nylon or an equivalent material. A diameter of the inner balloon ranges from 2 mm to 50 mm. In some embodiments, the inner balloon configuration matches the native vessel or a stent diameter, where the stent is positioned subsequently at the target site after removal of the plaque. The inner balloon is inflated using heated liquid formed by heating a fluid such as a saline. RF heating elements in the catheter are configured to introduce thermal energy within the inner balloon so that the inner balloon is configured to maintain a temperature range from 55° C. to 75° C. for at least 10 seconds(s) to 60 s. Higher the temperature, lower is the duration of thermal energy delivery to achieve the therapeutic outcome. In embodiments, thermocouple sensors and feedback mechanisms are integrated within the catheter to monitor and adjust balloon temperature in real-time to ensure uniform thermal distribution and prevent tissue damage.

The outer balloon is inflated using a 50/50 saline-contrast mixture to a pressure that is at least 0.1 atm higher than the inner balloon. Pressure within the outer balloon is kept within a range from 3 atm to 5 atm. The fluid (saline-contrast mixture) volume ranges from 1 mL to 50 mL depending on balloon size. A diameter of the outer balloon ranges from 2.2 mm to 75 mm. In some embodiments, the outer balloon is between 5% and 50% (or 1.1 to 1.5 times) larger in diameter than the inner balloon when both balloons are maximally inflated. In one embodiment, the outer balloon diameter is 1 mm greater than the inner balloon diameter. Moreover, in a length of the inner and outer balloons ranging from 5 mm to 80 mm, the outer balloon is at least 2 mm longer in length than the inner balloon. Outer balloon fluid volume ranges from 1 milli Liter (mL) to 50 mL depending on the size. While smaller volumes of the outer balloon reduce acoustic wave attenuation and improve energy delivery focus, larger volumes usually require multiple acoustic transducers and higher energy delivery to mitigate against acoustic wave attenuation to improve energy delivery.

Electrohydraulic emitters within the catheter deliver acoustic pressure pulses at an exemplary rate of 1 pulse per second (frequency of 1 Hz), through the saline-contrast mixture medium within the outer balloon. The emitters are positioned between the inner and the outer balloon to deliver acoustic energy to the fluid between the inner and outer balloon. The frequency of 1 Hz is applicable with peak pressure of 50 to 70 atm. Each pulse has an energy of approximately 3 kilo Volts (kV) which is discharged through a 220 nano Farad (nF) capacitor. Each pulse is delivered with a peak current of approximately 170 Ampere (A). In different embodiments, the pulse amplitude can be adjusted for varying plaque densities. During maximum inflation of the outer balloon, a diameter of the outer balloon is 1.1 to 1.5 times that of the inner balloon. These are exemplary parameters. Further discussion of the acoustic pulse energy parameters is provided below.

The double-balloon system improves acoustic pressure wave propagation by reducing the residual fluid volume between the inner and outer balloons. The volume of the fluid within the outer balloon defines the energy requirement of the pulses that are generated to create acoustic pressure waves. The residual fluid volume is obtained by subtracting the inner balloon volume from the outer balloon volume. The inner balloon displaces the fluid out of the outer balloon (which becomes a solid non-compliant structure) and hence less energy/lower inflation times are needed (which improve safety and efficacy). Additionally there is less need for multiple emitters for the IVL therapy and precise positioning of the emitters is possible resulting in a simpler and hence a smaller catheter structure. Smaller fluid volumes used with the double-balloon configuration, enhance thermal and mechanical energy transfer efficiency, enabling effective plaque fracture in shorter duration cycles. Experiments were performed with the described configurations, which demonstrated that reducing fluid volume within the outer balloon improved pulse efficiency by 63% for a 6 mm balloon and 74% for a 10 mm balloon.

The dual-mode catheter has two modes of operation: a thermal ablation mode for TBA and a lithotripsy mode for IVL. The thermal ablation mode generated controlled heating at the target site for plaque softening, and the lithotripsy mode applies acoustic pressure waves for plaque fracture. In an additional hybrid mode, the thermal ablation mode and the lithotripsy mode are operated simultaneously for improved dilation of a calcified plaque by simultaneously disrupting the calcium and elastin fibers.

The catheter's dual mechanism applies thermal energy to induce apoptosis in smooth muscle cells, remodeling elastin fibers while simultaneously using IVL shock waves to fracture calcified deposits. The synergistic effects result in superior luminal gain and long-term patency. A safety mechanism in the catheter configuration ensures an automatic shutdown in case of temperature deviation beyond ±2° C. or abnormal pressure fluctuations. Additionally, design of the catheter complies with FDA 510 (k) guidelines for vascular intervention devices.

In embodiments, short IVL cycle durations are adopted, which along with the double-balloon design, provide a significant advancement in minimizing ischemic risk while maintaining therapeutic effectiveness.

In the present specification, the terms generator, console, and treatment system may be used interchangeably to refer to such external apparatus. For clarity, the external apparatus is primarily referred to herein as the system, and the system may include one or more controllers, processors, power electronics, user interface components, sensors, pumps, valves, and associated circuitry or modules.

In various embodiments, the system is configured to control therapy based on one or more thermal targets. In some embodiments, the controller is configured to maintain a tissue-interface temperature within a target range (for example, a target interface temperature of about 55° C. to about 75° C., or other ranges as clinically appropriate) while limiting peak temperatures below a maximum safety threshold. In addition, or alternatively, the controller may be configured to deliver a prescribed thermal dose to the tissue. Thermal dose may be expressed, for example, as a cumulative equivalent minutes at 43° C. (CEM43), an Arrhenius damage integral, or another dose metric derived from measured and/or estimated interface temperature over time. The controller may adjust one or more parameters, such as heater power, heated fluid temperature, inner balloon pressure, outer balloon pressure, contact duty cycle, and/or treatment duration, to achieve the commanded interface temperature and/or commanded thermal dose.

Accordingly, in various embodiments the catheter system provides a programmable heat-flux pattern by selectively controlling contact and non-contact regions between coaxial balloons, thereby enabling localized thermal treatment with improved spatial selectivity, repeatability, and safety.

In various embodiments, the controller is configured to control therapy based on a prescribed thermal dose delivered to target tissue rather than, or in addition to, controlling to a temperature setpoint. Thermal dose may be expressed using any suitable dose metric. In some embodiments, thermal dose is expressed as cumulative equivalent minutes at 43° C. (CEM43). In some embodiments, thermal dose is expressed as an Arrhenius damage integral (Ω) or another time-temperature injury model. In other embodiments, thermal dose is expressed in joules. Thermal dose-based control may be used to achieve a desired therapeutic effect (e.g., remodeling and/or ablation of intimal and/or medial tissue) while limiting heating of non-target tissue (e.g., adventitia, blood and surrounding structures).

In some embodiments, the controller estimates and/or calculates the thermal dose based on one or more inputs including (i) one or more temperature measurements obtained from one or more temperature sensors positioned in or near the treatment region (for example, within the inner balloon, within the outer balloon, on a catheter shaft portion adjacent the balloons, or combinations thereof), (ii) exposure time corresponding to a duration of heating and/or a duration of a contact state between balloons, and (iii) one or more optional proxies for perfusion or convective heat sinking. By way of example, perfusion or flow proxy inputs may include pressure decay profiles, pressure-volume response, required heater power to maintain a temperature setpoint, thermal response time constants, or other parameters indicative of heat loss to blood flow and/or tissue perfusion.

In various embodiments, the controller uses the estimated thermal dose to automatically set, adjust, or terminate one or more therapy parameters. For example, the controller may adjust heater duty cycle and/or heater power to control a rate of dose accumulation. In addition, or alternatively, the controller may adjust a contact-state duty cycle between a contact state (reduced thermal resistance, higher heat flux) and a non-contact state (increased thermal resistance, lower heat flux) to shape the spatial and temporal distribution of delivered energy. In some embodiments, the controller sets a total treatment duration based on reaching a prescribed thermal dose threshold and terminates heating when the prescribed dose is achieved, optionally subject to safety limits including maximum allowable temperature, maximum allowable pressure, and/or maximum allowable inflation time.

In some embodiment, the thermal dose is calculated and predefined through, computer simulation models, bench-top or preclinical animal testing and further refined through clinical dosimetry.

Accordingly, thermal dose-based control provides a clinically meaningful and patient- and anatomy-adaptive control objective that improves treatment consistency and safety. By controlling to a prescribed total energy dose (rather than relying solely on time or a single temperature measurement), the system can deliver sufficient energy to achieve a therapeutic effect while reducing the likelihood of overheating and collateral thermal injury.

In some embodiments, the controller is configured to modulate the contact state between an inner balloon and an outer balloon to manage heat flux and reduce hotspot formation. For example, the controller may cycle between a contact state in which portions of the inner balloon contact portions of the outer balloon to provide relatively high heat flux, and a non-contact state in which a gap is present to provide reduced heat flux and allow thermal relaxation. Such cycling may be performed at a selected duty cycle and frequency to promote equilibration of local temperature variations and to reduce peak temperatures at localized contact points. The controller may adjust the duty cycle, cycle frequency, and/or pressure differential between balloons in response to measured temperatures and/or an estimated thermal dose accumulation rate.

In some embodiments, the system comprises one or more of the following components: (a) a first energy delivery subsystem configured to deliver electrical energy to a heating element associated with the catheter (for example, to heat a liquid within a heating chamber, within the catheter shaft, and/or within the inner balloon); (b) a fluid delivery subsystem configured to deliver and remove fluid from the inner balloon and the outer balloon, including independent control of inner balloon and outer balloon inflation states by activating and deactivating one or more pumps; (c) a sensing subsystem configured to obtain measurements from one or more sensors positioned in or around the catheter, including at least one temperature measurement and at least one pressure measurement; (d) a controller configured to execute programmatic instructions to regulate at least one of temperature, pressure, flow, volume, timing, and sequencing during a therapy workflow; (e) a second energy delivery subsystem configured to deliver electrical energy to one or more IVL emitters to generate acoustic energy deliverable via the outer balloon, (f) a user interface subsystem configured to display status and accept inputs (for example, a touch display, buttons, a footswitch input, and/or a remote interface); and (g) a data logging subsystem configured to store therapy data, alarms, and state transitions.

In some embodiments, the system comprises a console enclosure configured to house electronics and to provide a physical interface for user operation. The console enclosure may house the user interface (UI) subsystem, including a front panel with a display (e.g., a screen) and one or more user inputs. In some embodiments, the console includes a user interface board or UI computer configured to execute workflow software, coordinate subsystems, and present a guided therapy sequence (e.g., prime, inflate, heat/hold, deflate, repeat). The UI subsystem may accept one or more inputs including buttons, touchscreen inputs, and/or a footswitch input. In some embodiments, the console is powered from an AC mains source and includes internal power conversion to provide one or more DC rails for internal electronics and/or external modules.

In some embodiments, the fluid delivery subsystem comprises at least two pumps, including a first pump configured to deliver and withdraw fluid to and from the inner balloon and a second pump configured to deliver and withdraw fluid to and from the outer balloon. In other embodiments, the fluid delivery subsystem comprises fewer pumps (for example, a single multi-channel pump or a manifolded pump arrangement) or more pumps. In some embodiments, the pump units are external to the console and receive control commands from the console (e.g., over a wired communication link such as USB or other interface) while being powered independently (e.g., AC powered). In other embodiments, one or more pumps are integrated into the console enclosure. In some embodiments, any pump known in the art suitable for controlled balloon inflation may be used (including syringe pumps, piston pumps, peristaltic pumps, diaphragm pumps, and the like), and one or more valves, check valves, manifolds, vent/priming ports, filters, and/or degassing features may be included in the fluidic path(s).

In some embodiments, the system includes at least two fluid delivery channels configured to independently control the inner balloon and the outer balloon. In one non-limiting embodiment, the system is coupled to two separate pump units (e.g., two syringe pumps) each configured to drive a respective syringe and provide controlled fluid delivery through tubing to the catheter. For example, a first pump channel may be associated with the outer balloon fluid path to provide fixation and/or occlusion control, and a second pump channel may be associated with the inner balloon fluid path to deliver working fluid to be heated for therapy.

In some embodiments, the system interfaces with the catheter through at least one electrical connector and at least one fluid connector. In some embodiments, a single electrical connector carries conductors for energy delivery (for example RF energy) and at least one temperature sensor signal. In other embodiments, separate connectors are used to deliver electrical energy separately from the first energy delivery subsystem to the electrical circuit configured to heat liquid at the distal end of the catheter and from the second energy delivery subsystem to the IVL emitters.

In some embodiments, the system interfaces with at least two fluid paths, including an inner balloon fluid path and an outer balloon fluid path. In some embodiments, the system includes one or more manifolds, valves, check valves, vent/priming features, filters, and/or degassing features to support filling, priming, and repeatable operation.

In some embodiments, the sensing subsystem is configured to obtain at least one temperature measurement using one or more temperature sensors, including a thermocouple, RTD, thermistor, fiber optic temperature sensor, or combinations thereof. Temperature sensors may be positioned at any suitable location, including within the inner balloon, within the outer balloon, within a heating chamber, within a catheter shaft region, at a proximal connector region, or at any other location that provides a temperature indicative of working fluid temperature and/or tissue-interface temperature. In some embodiments, multiple temperature sensors are used to obtain a temperature profile, for example sensors spaced axially and/or circumferentially, and/or distributed sensing along a fiber. This sensing subsystem is optional and may not be used or included in situations where the catheter does not contain temperature sensors.

In some embodiments, the sensing subsystem is configured to obtain at least one pressure measurement. In some embodiments, the system includes an inner balloon pressure measurement and an outer balloon pressure measurement. In some embodiments, pressure is measured using pressure transducers in fluid communication with the inner balloon and outer balloon. In some embodiments, pressure sensing is implemented via an external hub or module configured to interface with sterile, disposable pressure transducers. In other embodiments, pressure transducers are integrated within the system and/or within disposable fluid sets. This sensing subsystem is optional and may not be used or included in situations where the catheter does not contain temperature sensors.

In some embodiments, the energy delivery subsystem comprises an RF energy delivery circuit configured to deliver RF energy to a heating element associated with the catheter to heat a working fluid. In some embodiments, the RF energy delivery circuit is configured for operation across a broad load impedance range to support variations in catheter design and working fluid composition. In some embodiments, the RF energy delivery circuit is configured to deliver power in a range of about 1 W to about 300 W, such as about 10 W to about 200 W, such as about 20 W to about 150 W, such as about 30 W to about 110 W, and may be configured to deliver voltage and current suitable for electrosurgical heating applications. In some embodiments, the system is configured with electrical isolation, leakage current control, and fault containment architectures consistent with requirements and constraints applicable to electrosurgical systems, while avoiding limitation to any particular certification status.

In some embodiments, the system is configured to deliver electrical energy to an electrode (or resistive heating element) in thermal communication with an ablative liquid, such that the power available at the electrode is a function of the total electrode-plus-cable impedance. The total impedance may be determined by a combination of factors including the electrical conductivity of the working fluid, the electrode geometry and surface area, the electrode-to-fluid interface condition, the catheter conductor geometry and length, connector interfaces, and temperature-dependent changes in fluid properties. In some embodiments, the system and the working liquid are jointly selected and/or controlled to position the operating impedance within a target impedance zone in which delivered power is maximized, thereby reducing time to heat the working liquid to a target temperature.

In some embodiments, the system is configured such that power available at the electrode increases as impedance approaches a target range near about 10 ohms and decreases outside that range. In some embodiments, the system and the working liquid are configured such that the operating impedance is maintained within a range of about 5 ohms to about 15 ohms for a first system configuration (System 1). In some embodiments, the system and the working liquid are configured such that the operating impedance is maintained within a range of about 5 ohms to about 30 ohms for a second system configuration (System 2). In some embodiments, the system is configured to provide its maximum or near-maximum deliverable power when the operating impedance is approximately 10 ohms (for example, within ±50% of 10 ohms, within ±30% of 10 ohms, or within ±20% of 10 ohms).

In some embodiments, rapid heating of the working liquid is desirable, and faster heating is preferred. Accordingly, in some embodiments the system is configured to deliver a higher power level at the electrode when the impedance is within the target impedance zone to reduce time to reach a temperature setpoint (e.g., about 65° C., optionally selectable within about 50° C. to about 80° C. with closed-loop tolerance such as +2° C. as described elsewhere herein). In some embodiments, the system is configured such that time to reach the target temperature is reduced as compared to operation outside the target impedance zone, for example by providing a power peak or plateau in the target zone. In some embodiments, time to reach the target temperature is less than about 60 seconds, less than about 30 seconds, less than about 20 seconds, less than about 10 seconds, or less than about 5 seconds, depending on balloon volume, starting temperature, working liquid composition, and electrode configuration. These values are non-limiting examples and may vary across catheter sizes and treatment protocols.

In some embodiments, the working liquid comprises saline, contrast, and mixtures thereof, and the system is configured to accommodate impedance variations arising from different mixture ratios and temperature-dependent conductivity changes. For example, a more conductive working liquid (e.g., higher saline fraction) may yield lower impedance and may shift the operating point toward the target impedance zone, while a less conductive working liquid (e.g., higher contrast fraction) may yield higher impedance and may shift the operating point away from the target impedance zone. In some embodiments, the system is configured to support operation with working liquids spanning a broad impedance range (for example from below about 5 ohms to above about 100 ohms, and in some embodiments to above about 300 ohms), while still providing a preferred operating region near the target impedance zone.

In some embodiments, the working liquid is selected, prepared, and/or adjusted during use to achieve a target impedance at the electrode, including by selecting a saline-to-contrast ratio, selecting a base liquid (e.g., saline, water, buffered solution), selecting additives, controlling temperature, and/or controlling mixing. In some embodiments, the working liquid is mixed or circulated to improve impedance uniformity and heating uniformity. In some embodiments, the system includes one or more of: an output stage, matching network, transformer, filter network, switching network, and/or control algorithms configured to shape the delivered power versus impedance response so that maximum or near-maximum power is available within a desired impedance range.

The system is preferably configured to deliver controlled heating power across a broad electrode-plus-cable impedance range, including at least from about 5 ohms to about 100 ohms, and in some embodiments up to about 300 ohms or more, to support different working liquids, catheter designs, and procedural conditions. The system is preferably configured to provide a maximum or near-maximum deliverable power within a target impedance zone near about 10 ohms, including: about 5-15 ohms for a first system configuration; and/or about 5-30 ohms for a second system configuration. The system is preferably configured so that, when the operating impedance is within the target impedance zone, the delivered power is higher than when the operating impedance is outside the target impedance zone, thereby enabling faster heating of the working fluid and reduced time-to-target temperature.

Because impedance may change during a treatment cycle (e.g., as temperature rises and conductivity changes), the system may measure impedance (directly or indirectly) and may adjust one or more of output voltage, output current, duty cycle, waveform parameters, and/or matching to maintain effective heating. In some embodiments, the controller uses impedance estimation to select a power command that achieves the fastest allowed heating while maintaining safety limits.

The system may provide recommended working liquid compositions or mixing protocols that place the operating impedance in the target impedance zone for the selected catheter and treatment profile, thereby maximizing available electrode power and reducing time-to-target temperature.

In some embodiments, the controller: estimates operating impedance from measured voltage/current and uses the estimate to select a power setpoint or duty cycle; logs impedance and delivered power versus time as part of the treatment record; detects off-target impedance (too low or too high) and prompts a liquid adjustment (e.g., adjust saline/contrast ratio) and/or automatically adapts output to maintain heating performance.

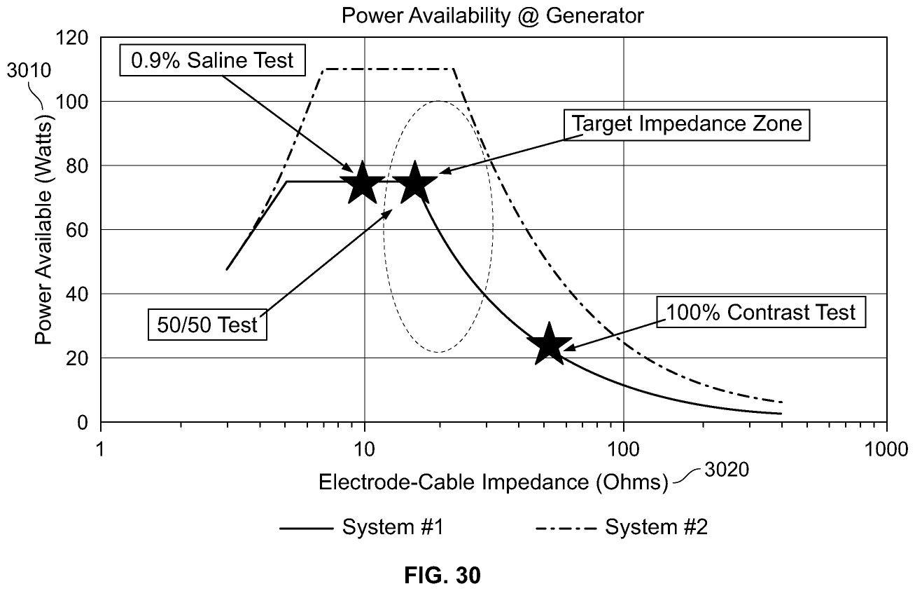

In some embodiments, the power available 3010 is a function of electrode plus cable impedance 3020 and may be optimized by selecting a target saline/contrast mix as shown in FIG. 30.

In some embodiments, the controller is configured to provide closed-loop regulation of (i) inner balloon pressure, (ii) outer balloon pressure, and (iii) working fluid temperature and/or tissue-interface temperature. In some embodiments, the controller executes at least the following control loops: (a) an outer balloon pressure loop using an outer balloon pressure signal and pump/valve actuation to achieve a commanded outer balloon inflation state; (b) an inner balloon pressure loop using an inner balloon pressure signal and pump/valve actuation to achieve a commanded inner balloon inflation state; and (c) a temperature loop using at least one temperature signal to regulate energy delivery to achieve a commanded temperature. In some embodiments, the temperature loop regulates to a target temperature setpoint of about 65° C. In some embodiments, the target temperature is selectable within a range of about 50° C. to about 80° C., including any subrange therein. In some embodiments, the controller maintains a measured temperature within about ±2° C. of the selected setpoint (for example, by modulating RF power and/or duty cycle). In some embodiments, the controller additionally or alternatively controls to a thermal dose metric as described elsewhere herein (for example CEM43 or an Arrhenius-based dose metric), without requiring limitation to any particular model.

In some embodiments, the inner balloon and outer balloon are controlled to operate at pressures within a preferred operating range of about 2 atm to about 10 atm, including any subrange therein. In some embodiments, the system is configured to support pressures outside the preferred range, including up to about 100 atm for certain test, calibration, or non-limiting embodiments. In some embodiments, a nominal target pressure is about 8 atm.

In one non-limiting example, the controller is configured to execute a therapy cycle having an inflation period of about 30 seconds. The cycle may include a ramp interval of about 5 seconds during which one or both balloons are increased toward a target pressure (for example about 8 atm) and a thermal setpoint (for example about 65° C.), a hold interval of about 20 seconds during which pressure and temperature are maintained (for example about 8 atm and about 65° C.), and a deflation interval of about 5 seconds during which one or both balloons are deflated. In some embodiments, the inner balloon deflation completes before the outer balloon deflation, for example by about 1 second, to support controlled disengagement, pressure management, and/or reperfusion.

In some embodiments, the system is configured to log data including one or more of: timestamps, commanded pressures, measured pressures, commanded energy output, measured/delivered energy output, temperature measurements, alarms/faults, and workflow state transitions. In some embodiments, the system exports data in a machine-readable format (for example CSV) and/or a human-readable session report.

In some embodiments, the system includes one or more interlocks to reduce the likelihood of unintended energy delivery or unintended pump actuation. In some embodiments, catheter identification and/or keying is implemented as needed based on functional requirements. Catheter identification and/or keying may include, for example, mechanical keying, resistor coding, memory devices (for example an EEPROM), connector pin patterns, RFID, or other identification approaches. In some embodiments, catheter ID is used to enable, disable, or configure therapy modes, therapy limits, or workflow parameters.

In some embodiments, the system is configured to support additional intravascular therapies using shared infrastructure, including shared controller, shared user interface, shared data logging, shared safety management, and shared pressure control. In some embodiments, the system includes one or more additional energy delivery subsystems and associated outputs in addition to the hydrothermal RF heating stage.

In some embodiments, the catheter comprises one or more pulsed field ablation electrodes incorporated at a distal end of the catheter (for example on a distal electrode assembly, on a shaft region, and/or on or adjacent a balloon), and the system is configured to deliver pulsed electric field energy to the one or more electrodes. In some embodiments, the system comprises a pulsed energy delivery stage configured to deliver programmable pulse trains including selectable pulse count, pulse width, pulse amplitude, inter-pulse spacing, and polarity, including bipolar and/or biphasic pulses. In some embodiments, the system monitors delivered voltage and current and disables output under fault conditions.

In some embodiments, the system comprises an RF energy subsystem (e.g., an RF power board) configured to deliver electrical energy to a catheter heating element to heat a working fluid associated with the inner balloon. In some embodiments, the RF energy subsystem includes thermocouple measurement circuitry and provides one or more feedback signals to the controller to enable closed-loop temperature regulation (e.g., regulation to a setpoint such as about 65° C., optionally selectable within about 50-80° C., with a closed-loop tolerance such as ±2° C. as described elsewhere herein). In some embodiments, the RF energy subsystem includes a single catheter electrical interface configured to provide both RF energy delivery conductors and temperature sensor conductors (e.g., thermocouple conductors) to the catheter. In other embodiments, the electrical interface is split among multiple connectors, or temperature sensing is provided through a separate interface.

In some embodiments, the RF energy subsystem is configured to operate across a broad impedance range to accommodate different working liquids (e.g., saline/contrast mixtures) and catheter evolutions, and may include power, current, and voltage capabilities consistent with electrosurgical design constraints. The specific values shown in any diagram are non-limiting examples and may be implemented as “about,” “up to,” and/or “at least” ranges to maintain design flexibility.

In some embodiments, the system comprises an instrumentation subsystem configured to interface one or more pressure transducers for measuring pressure in the inner balloon and/or the outer balloon. In one non-limiting embodiment, the instrumentation subsystem is implemented as an external instrumentation module that is communicatively coupled to the console (e.g., via a wired interface such as USB or other data link) and receives electrical power from the console and/or an external source. The external instrumentation module may include signal conditioning and/or analog-to-digital conversion for one or more pressure transducer signals, and may provide the measured pressure signals to the controller for closed-loop regulation.

In some embodiments, the pressure transducers are sterile disposable pressure transducers coupled to the fluid lines through suitable fittings, and the instrumentation subsystem is configured to receive signals from at least two pressure transducers, including a first pressure transducer associated with an inner balloon fluid path and a second pressure transducer associated with an outer balloon fluid path. In some embodiments, pressure control is executed by the UI computer/controller using the measured pressure signals and pump actuation commands (for example, to maintain pressures within a preferred range of about 2-10 atm and optionally support higher maximum pressures up to about 100 atm as described elsewhere herein). In other embodiments, pressure control is executed by a dedicated pressure controller (integrated in the console or external module), and the UI computer provides supervisory commands.

In some embodiments, the catheter is coupled to the system through (i) fluid tubing connecting the pump subsystem(s) and pressure transducer locations to the catheter fluid connectors, and (ii) an electrical connection configured to provide RF energy and temperature sensing signals between the console and the catheter. In some embodiments, the system routes the RF and temperature sensing through a single catheter electrical interface. In some embodiments, the system includes strain reliefs, keyed connectors, and/or optional catheter identification features; catheter ID/keying may be included where driven by functional needs (e.g., modality enablement, limiting, logging) but is not required in all embodiments.

In another embodiment, the instrumentation and/or pump units may be integrated into the console enclosure, distributed among multiple enclosures, or implemented as modular subsystems. For example, the pressure transducer interface may be integrated into the console, the pumps may be integrated into the console, or the system may include additional modules (e.g., for IVL and/or PFA) while retaining shared UI, logging, safety interlocks, and workflow control.

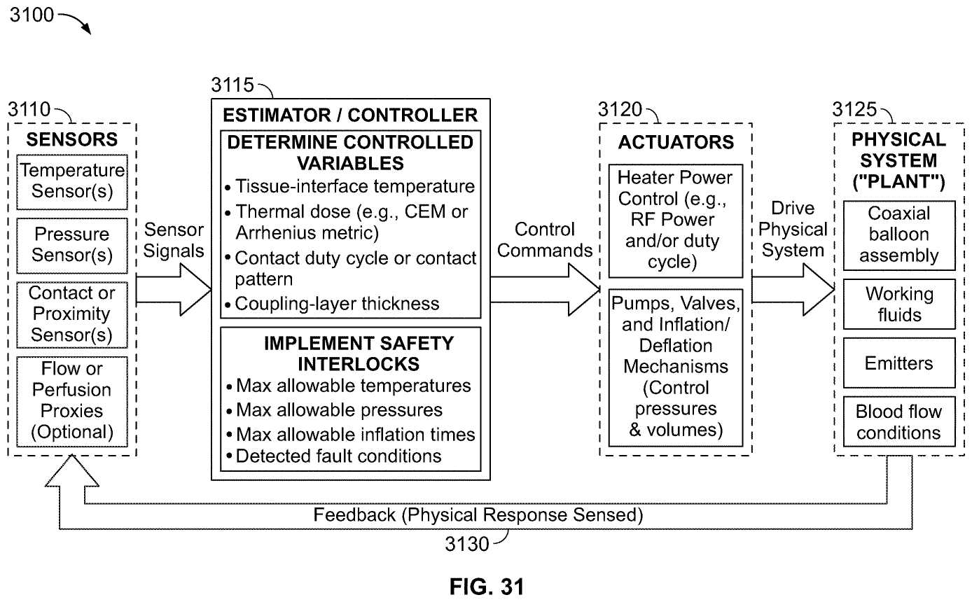

Referring to FIG. 31, an example closed-loop control architecture 3100 configured for controlling a therapeutic system, such as a coaxial balloon assembly used for vascular treatments, is shown. The architecture 3100 is designed to manage various treatment modalities, including thermal therapy (e.g., remodeling of tissue via heat), lithotripsy therapy (e.g., fracture of calcified lesions via acoustic shockwaves), and/or combined modes thereof. In one embodiment, the control architecture 3100 comprises a sensing subsystem 3110 configured to monitor the state of the system and the treatment environment. The sensing subsystem 302 may is optional and may generate one or more sensor signals based on detected physical parameters. These sensors may include, but are not limited to: a) temperature sensors: thermocouples, thermistors, or fiber optic sensors positioned to measure the temperature of the ablative liquid, the balloon wall, or the tissue interface, b) pressure sensors: transducers configured to measure inflation pressure within the balloon(s) or acoustic pressure waves generated during lithotripsy, c) contact or proximity sensors: impedance sensors or capacitive sensors configured to determine if the therapeutic element is in proper apposition with the vessel wall, d) flow or perfusion proxies: sensors configured to estimate blood flow rates or cooling effects caused by perfusion surrounding the treatment site.

The sensor signals are transmitted to an estimator/controller 3115. The controller 3115 may be implemented via a microcontroller, FPGA, ASIC, or general-purpose processor executing control logic. The controller 3115 performs two primary logical functions: variable determination and safety interlocks. The controller 3115 processes input signals to calculate or estimate controlled variables that cannot always be measured directly. These may include the tissue-interface temperature, a thermal dose metric (e.g., a cumulative equivalent minutes (CEM) calculation or an Arrhenius-based damage integral), a contact duty cycle or pattern (optimizing when energy is applied based on vessel movement), and/or a coupling-layer thickness (ensuring adequate fluid coupling for shockwave transmission).

Concurrently, the controller 3115 implements safety interlocks to ensure patient safety. These interlocks compare sensor data against pre-determined thresholds, such as maximum allowable temperatures (to prevent charring or thrombosis), maximum allowable pressures (to prevent vessel dissection or balloon rupture), maximum inflation times (to prevent ischemia), and other detected fault conditions (e.g., sensor disconnection).

Based on the processing of the sensor signals and the safety constraints, the controller 3115 generates specific control commands. These commands are transmitted to an actuator subsystem 3120. The actuator subsystem 3120 includes the hardware drivers required to manipulate the energy and fluid delivery. Heater power control: may comprise modulating an RF generator, adjusting voltage/current, or modifying the duty cycle of an electrical heating element. Pump, valve, and/or inflation mechanisms control the ingress and egress of working fluids to regulate the pressure and volume of the coaxial balloon assembly, thereby controlling the mechanical expansion and the acoustic coupling medium.

The actuator subsystem 3120 drives the physical system (or “Plant”) 3125. In the context of control theory, the “Plant” represents the physical entity being controlled. As illustrated, this includes the coaxial balloon assembly (the catheter device), the working liquid (e.g., saline, contrast mixture), IVL emitters, and the blood flow conditions (which act as a thermal load or heat sink).

As indicated by the bottom arrow in FIG. 31, the architecture relies on feedback 3130. The actions taken by the actuators (e.g., heating the fluid, expanding the balloon) result in a physical response within the Plant 3125. This response (e.g., a rise in fluid temperature, a change in pressure) is immediately detected by the sensing subsystem 3110. This closes the loop, allowing the controller 3100 to dynamically adjust energy delivery rates, switch between contact and non-contact states, and regulate therapy timing to maintain the target setpoints despite disturbances in the biological environment.

In some embodiments, the catheter comprises one or more IVL emitters positioned within or coupled to the outer balloon, and the system is configured to deliver electrical drive signals to energize the IVL emitters to generate acoustic pressure waves. In some embodiments, the system is configured to deliver IVL pulses at a programmable repetition rate; in one non-limiting embodiment, the IVL pulse repetition rate is about 1 Hz. In some embodiments, the system tracks delivered pulse counts and may enforce pulse limits and/or lockouts as desired.

In various embodiments, the system is configured to improve lithotripsy performance and repeatability by defining and controlling one or more lithotripsy delivery metrics, by treating emitter placement and actuation as controllable variables, and/or by coordinating lithotripsy delivery with thermal conditions within the balloon system.

In some embodiments, the system utilizes a metric referred to herein as “pulse efficiency” to characterize delivery of lithotripsy energy to target tissue. Pulse efficiency may be defined as a measure of acoustic output and/or effective mechanical effect produced at or near the target region per emitted pulse, or per unit input energy. In various embodiments, pulse efficiency may be quantified using one or more measurement methods including: (i) measurement of peak pressure and/or pressure waveform characteristics at or near a balloon wall using a hydrophone or other pressure sensor, (ii) measurement of fracture incidence and/or fracture extent in a calcified lesion surrogate such as a calcium phantom subjected to a prescribed number of pulses, (iii) determination of energy required per fracture event or per unit fracture extent, and/or (iv) estimation of an acoustic attenuation or coupling proxy derived from measured pressure waveforms, impedance changes, or other sensed parameters indicative of acoustic transmission through a coupling medium. In some embodiments, the controller records pulse efficiency and adjusts operating parameters to improve or maintain pulse efficiency during a procedure.

In various embodiments, emitter positioning and actuation are treated as controlled design and operating variables. In some embodiments, the outer balloon includes a plurality of lithotripsy emitters that are axially staggered along the balloon length to increase spatial coverage and reduce interference patterns. In some embodiments, the emitters are independently addressable such that the controller can select subsets of emitters for activation based on lesion length, vessel diameter, or measured response. In some embodiments, the controller fires emitters according to a predetermined or adaptive sequence configured to reduce the formation of standing-wave nulls, reduce localized overexposure, and/or increase uniformity of acoustic energy distribution. The sequence may include alternating emitters, rotating activation among emitters, phase-shifted firing, or other sequencing strategies.

In certain embodiments, the system coordinates lithotripsy delivery with thermal conditions to improve overall therapy performance. In some embodiments, lithotripsy pulses are delivered during a defined thermal window, for example after an inner balloon working fluid reaches a target temperature range, or while the outer balloon coupling medium is maintained within a selected temperature range, such that acoustic coupling properties and/or tissue mechanical properties are within a desired range. In other embodiments, the controller selects alternation between lithotripsy delivery and thermal delivery based on measured compliance or mechanical response of the treated segment, such as a pressure-volume response, balloon compliance changes during inflation, or other indicators of lesion modification. In this manner, the system can co-optimize thermal and acoustic delivery to improve efficiency and controllability of treatment, rather than merely sequencing two therapies without feedback. Table 1 lists IVL pulse parameters with outcome ranges.

TABLE 1
IVL pulse parameters and outcome ranges

| | | | | Preferred | | |
|---|---|---|---|---|---|---|
| Category | Parameter | Range | | Range | | Description |
| Pulse | Pulse repetition frequency | 0.1-100 | Hz | 0.25-10 | | PRF may be fixed |
| timing | (PRF) | | | Hz; 0.5-5 | | variable, or adaptive |
| | | | | Hz; 1 Hz | | during a pulse train. |
| Pulse | Pulses per burst/train | 1-500 | pulses | 5-50; 10 | | “Burst” can be a |
| timing | | | | pulses | | contiguous train or |
| | | | | | | grouped sub-trains. |
| Pulse | Inter-burst interval | 0-300 | s | 0.5-30 | s | Enables thermal |
| timing | | | | | | relaxation, |
| | | | | | | reperfusion, and/or |
| | | | | | | repositioning |
| Pulse | Total pulses per | 1-5000 | | 10-500; | | Trackable by |
| timing | procedure or catheter use | pulses | | 20-200 | | controller; may be |
| | | | | | | limited by catheter |
| | | | | | | ID or internal |
| | | | | | | counter. |
| Electrical | Electrical energy | 0.01-200 | J | 0.1-50 J; | | “Delivered energy” |
| drive | delivered per pulse (to | | | 0.5-10 J | | can be computed |
| | emitter circuit) | | | | | from V · I · dt, |
| | | | | | | capacitor discharge |
| | | | | | | energy, or calibrated |
| | | | | | | model. |
| Electrical | Electrical peak voltage (at | 50 V-10 kV | | 200 V-5 | | Depends on emitter |
| drive | generator output or at | | | kV | | topology and cable |
| | emitter terminals) | | | | | loss. |
| Electrical | Electrical peak current | 0.1-200 | A | 1-80 | A | Sufficient to energize |
| drive | | | | | | emitter. |
| Electrical | Electrical drive pulse | 0.05 μs-10 | | 0.5-200 | | The electrical event |
| drive (pulse | width (full-width or | ms | | μs; 1-50 | | driving the emitter; |
| width) | effective discharge | | | μs | | distinct from the |
| | duration) | | | | | acoustic waveform |
| | | | | | | duration. |
| Acoustic | Acoustic peak pressure | 0.1-50 | MPa | 1-20 MPa; | | Can be measured |
| output | amplitude at/near balloon | | | 2-10 MPa | | using a hydrophone |
| | wall (or “peak positive | | | | | in water tank, PVDF |
| | pressure”) | | | | | sensor, calibrated |
| | | | | | | phantom sensor |
| Acoustic | Peak-to-peak pressure | 0.2-100 | MPa | 2-40 | MPa | “Peak” and “peak- |
| output | amplitude | | | | | to-peak.” |
| Acoustic | Acoustic pulse duration | 0.1-200 | μs | 0.5-50 | μs | Full-width at half |
| output | (e.g., FWHM of pressure | | | | | maximum, 10-90%, |
| (pulse | waveform) | | | | | (energy-containing |
| width) | | | | | | duration). |
| Acoustic | Acoustic energy per pulse | 0.001-20 | J | 0.01-5 | J | Definable via |
| output | at target region | | | | | calibrated transfer |
| | (mechanical/acoustic) | | | | | function or proxy |
| | | | | | | metrics. |
| Balloon | Balloon inflation pressure | 0.5-30 | atm | 2-10 atm; | | Higher than |
| state during | during IVL pulsing | | | 4-8 atm | | conventional ablative |
| IVL | | | | | | pressures |
| System | Pulse efficiency (defined | 1-99% | | 20-90% | | It is a ratio: measured |
| control | metric) | | | | | peak pressure vs |
| | | | | | | input energy; |
| | | | | | | fractures per joule; or |
| | | | | | | pressure transmission |
| | | | | | | through coupling |
| | | | | | | layer. |
| Outcomes: | Fracture incidence | 1-100% | | 20-100% | | Defined for a |
| fracture | (percent of tests | | | | | standard phantom or |
| | showing ≥1 fracture) | | | | | calcified segment; |
| | | | | | | measured by imaging |
| | | | | | | (micro-CT, OCT, |
| | | | | | | fluoroscopy) or direct |
| | | | | | | inspection. |
| Outcomes: | Fractures per pulse (mean | 0-10 | | 0.01-2 | | Some pulses fracture, |
| fracture | or median) | | | | | others don't. |
| Outcomes: | Fractures per burst/train | 0-500 | | 1-50 | | Fractures over a |
| fracture | | | | | | pulse burst cycle (not |
| | | | | | | just one pulse) |
| Outcomes: | Energy per fracture event | 0.01-500 | J | 0.1-50 | J | Indicative of an |

| | | | | |
|---|---|---|---|---|
| fracture | (J/fracture) | | | efficiency level of |
| | | | | the treatment |
| Outcomes: | Change in compliance | 0.5-10× | 1.1-5× | Measured via |
| plaque | (e.g., ΔdV/dP or stiffness | improvement | | pressure-volume |
| mechanics | proxy) | | | response, diameter |
| | | | | change vs pressure, |
| | | | | or compliance index |
| | | | | before/after pulses. |
| Outcomes: | Lumen gain (acute) | 0-50% | 1-20% | Measured by |
| lumen/ | | | | angiography, |
| imaging | | | | IVUS/OCT |
| | | | | diameter/area change |

In some embodiments, the system is configured to deliver IVL energy as a sequence of pulses having a programmable pulse repetition frequency in a range from about 0.1 Hz to about 100 Hz (for example about 0.5 Hz to about 10 Hz, and in some embodiments about 1 Hz), with a pulse count per pulse train in a range from about 1 to about 500 pulses (for example about 5 to about 50 pulses, and in some embodiments about 10 pulses). In some embodiments, the system is configured to deliver a total number of pulses during a procedure and/or per catheter in a range from about 1 to about 5000 pulses (for example about 10 to about 500 pulses), with optional inter-burst intervals from about 0 seconds to about 300 seconds.

In some embodiments, each IVL pulse corresponds to an electrical discharge event delivered to one or more emitters disposed in or associated with a balloon, wherein electrical energy delivered per pulse is in a range from about 0.01 J to about 200 J (for example about 0.1 J to about 50 J). In some embodiments, the electrical drive pulse width is in a range from about 0.05 microseconds to about 10 milliseconds (for example about 0.5 microseconds to about 200 microseconds). In some embodiments, the resulting acoustic pressure waveform has a peak pressure amplitude at or near a balloon wall in a range from about 0.1 MPa to about 50 MPa (for example about 1 MPa to about 20 MPa) and an acoustic pulse duration in a range from about 0.1 microseconds to about 200 microseconds (for example about 0.5 microseconds to about 50 microseconds), as measured using a hydrophone or other pressure sensor in a defined test configuration.

In some embodiments, the system is configured to quantify IVL performance using one or more outcome metrics including peak pressure at the balloon wall, fracture incidence, fracture count, energy per fracture event, and/or compliance change. In some embodiments, fracture incidence is in a range from about 1% to about 100% and fracture count is in a range from about 0 to about 500 fractures per pulse train, depending on plaque composition, coupling conditions, and pulse dosing. In some embodiments, the controller logs delivered pulse count, pulse parameters, and one or more measured or estimated efficiency metrics (e.g., fractures per joule, pressure transmission proxy, or peak pressure per delivered energy) as part of a treatment record.

In some embodiments, the present disclosure defines electrical pulse width and acoustic pulse width as separate, independently claimable parameters, and further defines peak pressure using multiple equivalent metrics. Any one of the definitions below may be used alone or in combination, and the system may be configured to determine, estimate, control, and/or log any of these metrics.

“Electrical pulse width” refers to a duration of an electrical drive event applied to an IVL emitter, and may be defined in any of the following non-limiting ways:
1. Full-width at half maximum (FWHM) of the emitter drive voltage waveform, defined as the time interval during which instantaneous voltage magnitude is at least 50% of a peak magnitude.
2. Full-width at half maximum (FWHM) of the emitter drive current waveform.
3. An energy-containing duration, defined as a shortest contiguous time interval that contains at least a selected fraction of delivered electrical energy (for example at least 80%, at least 90%, at least 95%, or at least 99%) as computed by integrating V(t)·I(t) over time.
4. A threshold-crossing duration, defined as the time interval during which voltage magnitude exceeds a threshold (for example 10%, 20%, or 30% of peak, or an absolute threshold), or during which current exceeds a threshold.
5. A capacitor discharge duration, defined as a time interval between a first time when a capacitor voltage crosses a first threshold and a second time when the capacitor voltage crosses a second threshold (e.g., between 90% and 10% of initial discharge voltage), for embodiments that use capacitor discharge drive.

In some embodiments, electrical pulse width is in a range from about 0.05 microseconds to about 10 milliseconds, including any subrange therein (for example about 0.5 microseconds to about 200 microseconds), depending on emitter type, drive topology, and desired acoustic output.

“Acoustic pulse width” refers to a duration of an acoustic pressure waveform produced by an IVL emitter, and may be defined in any of the following non-limiting ways:
1. FWHM of a pressure waveform, defined as the time interval during which pressure magnitude exceeds 50% of a peak pressure magnitude.
2. A peak-to-peak event duration, defined as the time interval from a first extremum (e.g., peak positive pressure) to a subsequent extremum (e.g., peak negative pressure), or between successive zero-crossings around a principal pulse lobe.
3. An energy-containing duration, defined as a shortest contiguous time interval that contains at least a selected fraction of acoustic energy, where acoustic energy is estimated from the pressure waveform using a calibrated transfer function or proxy metric.
4. A threshold-crossing duration, defined as the time interval during which pressure magnitude exceeds a threshold (for example 10%, 20%, or 30% of a peak magnitude, or an absolute threshold such as a selected MPa value).

In some embodiments, acoustic pulse width is in a range from about 0.1 microseconds to about 200 microseconds (for example about 0.5 microseconds to about 50 microseconds), including any subrange therein.

“Peak pressure” at or near a balloon wall, or at a defined measurement location, may be reported using any one or more of the following equivalent metrics:
1. Peak positive pressure (P+), defined as the maximum instantaneous pressure above ambient during a pulse.
2. Peak negative pressure (P−), defined as the minimum instantaneous pressure below ambient during a pulse.
3. Peak-to-peak pressure (Ppp), defined as P+ minus P−.
4. RMS pressure over a defined time window (PRMS), for example RMS over the acoustic pulse width window or over a fixed window length (e.g., 1-200 microseconds).
5. A windowed peak metric, defined as a maximum of a filtered pressure waveform (e.g., band-limited or low-pass filtered) to reduce sensitivity to sensor resonance, ringing, or high-frequency noise.
6. A spatially referenced peak metric, defined as the maximum pressure measured at a specified distance from the balloon surface (e.g., at the balloon wall, at 0.5 mm, 1 mm, 2 mm, 5 mm, or other defined distance), optionally corrected using a calibration model to estimate pressure at the balloon wall.

In some embodiments, peak pressure amplitude (as any of the metrics above) is in a range from about 0.1 MPa to about 50 MPa, including any subrange therein (for example about 1 MPa to about 20 MPa).

In some embodiments, one or more of the above metrics are obtained using a hydrophone in a water tank, a PVDF sensor, a fiber optic pressure sensor, or another pressure sensor, with the sensor positioned at a defined location relative to a balloon surface. In some embodiments, the system uses a calibrated model to infer balloon-wall pressure from a measurement taken at a non-zero distance from the balloon. In some embodiments, the system measures delivered electrical energy per pulse at the generator output and/or at the emitter terminals and stores the delivered energy and one or more pressure metrics as part of a treatment record.

In some embodiments, the controller is configured to: (a) determine or estimate electrical pulse width using one or more of the electrical definitions above; (b) determine or estimate acoustic pulse width using one or more of the acoustic definitions above; (c) determine or estimate at least one peak pressure metric (P+, P−, Ppp, PRMS, filtered peak, or spatially referenced peak); (d) adapt one or more pulse parameters (pulse repetition frequency, pulse count, pulse energy, and/or pulse width) based on the determined or estimated metrics; and/or (e) log one or more of pulse width metrics and peak pressure metrics along with delivered pulse count and delivered energy per pulse.

In some embodiments, the system characterizes IVL pulse delivery using one or more electrical metrics, one or more acoustic metrics, and/or one or more outcome metrics, and any such metric may be used interchangeably as a control target, a specification parameter, and/or a logged treatment record variable. Accordingly, unless expressly stated otherwise, a “pulse parameter” as used herein includes an electrical-domain pulse parameter (e.g., electrical pulse width, delivered electrical energy per pulse, peak voltage, peak current, or repetition frequency), an acoustic-domain pulse parameter (e.g., acoustic pulse width, peak positive pressure, peak negative pressure, peak-to-peak pressure, RMS pressure over a defined window, filtered peak pressure, or spatially referenced peak pressure), and/or a derived parameter computed from one or more measured signals (e.g., a normalized pressure metric, a pressure transmission proxy, a pressure-per-energy metric, an energy-containing duration, or a threshold-crossing duration). Any of the foregoing may be used alone or in combination, and the system may be configured to use different definitions at different times, for different catheters, or for different anatomical targets.

In some embodiments, an electrical pulse width definition and an acoustic pulse width definition are treated as alternative representations of a “pulse width,” and either may be used to specify, control, or verify therapy delivery. For example, a controller may regulate an electrical discharge duration based on a measured drive waveform (e.g., voltage or current) while separately monitoring an acoustic waveform duration, and either metric may be used to confirm that a commanded pulse was delivered as intended. In some embodiments, acoustic pulse width is inferred or estimated from electrical drive parameters using a calibration model, and the inferred acoustic pulse width is used as a substitute for direct acoustic measurement. Conversely, in some embodiments, the electrical pulse width is adapted in response to a measured or estimated acoustic pulse width.

In some embodiments, any peak pressure metric may be substituted for any other peak pressure metric. For example, peak positive pressure, peak-to-peak pressure, RMS pressure over a defined window, filtered peak pressure, or spatially referenced peak pressure may be used as a proxy for the others, and the controller may be configured to translate among these metrics using calibration curves, transfer functions, and/or empirically derived correlations. In some embodiments, a peak pressure metric measured at a non-zero distance from a balloon surface is converted to an estimated balloon-wall peak pressure using a model, and either the measured value or the estimated value may be used as the “peak pressure” for specification, control, and/or logging.

In some embodiments, outcome metrics are treated as equivalent or substitutable therapy-performance indicators. For example, one or more of fracture incidence, fracture count, fractures per pulse, fractures per joule, energy per fracture event, and compliance change (or a pressure-volume response proxy) may be used as an outcome metric, and any outcome metric may be used to adjust or optimize one or more pulse parameters. In some embodiments, a controller modifies pulse repetition frequency, pulse count, delivered energy per pulse, and/or electrical pulse width responsive to a measured or estimated peak pressure metric and/or an outcome metric, and the system logs the pulse parameters and the metric(s) used to determine delivered therapy.

In some embodiments, where direct measurement of an acoustic metric (e.g., pressure waveform) or an outcome metric (e.g., fracture count) is not available in vivo, the system uses a proxy measurement and/or model-based estimate as a substitute. Non-limiting proxy measurements include voltage/current-based delivered energy estimates, impedance-based coupling estimates, pressure-volume response-based compliance estimates, and/or time-domain features of sensed signals. In some embodiments, the system stores either the direct metric, the proxy metric, the inferred metric, or any combination thereof as part of a treatment record.

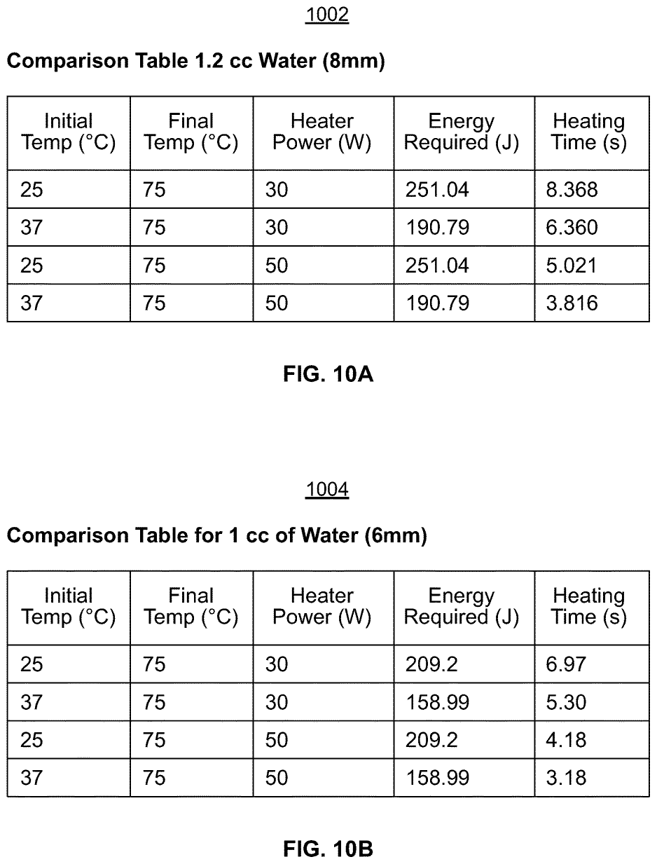

FIG. 10A illustrates a comparison table 1002 for heating time required to heat 1.2 cc of water at different initial temperatures, using a first power of 30 W and a second power of 50 W, to achieve the final temperature of 75° C. FIG. 10B illustrates another comparison table 1004 for heating time required to heat 1 cc of water at different initial temperatures, using a first power of 30 W and a second power of 50 W, to achieve the final temperature of 75° C. It is desirable to have ablative time <30 seconds and is ideal to have the ablation time <15 second. In some embodiments the ablation times are <10 seconds. It is desirable to have ablative temperature >50° C. and is ideal to have the ablative temperature ~65° C.+/−5° C. In some embodiments the ablative temperature >75° C. In most embodiments the ablative temperature is <100° C. and in no embodiment the ablative temperature exceeds 110° C. for more than 1 second.

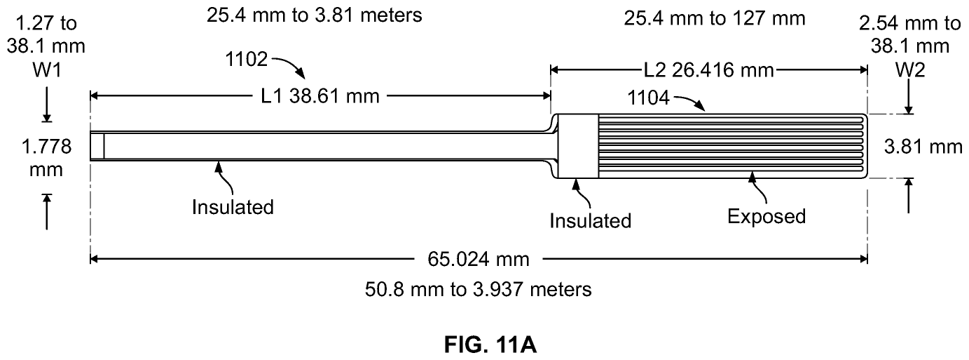

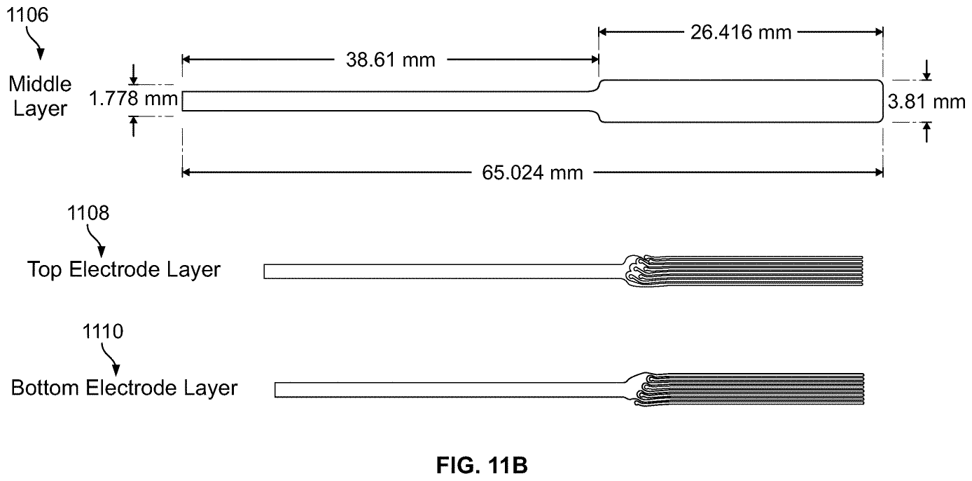

FIG. 11A illustrates an exemplary design of an electrode configuration used for heating an ablation fluid for thermal ablation in accordance with some embodiments of the present specification. The design comprises an elongated rectangular body with a first portion 1102 having a first length L1 ranging from 25.4 mm to 3.81 meters, and is 38.61 mm in an embodiment, and having a first width W1 ranging from 1.27 to 38.1 mm, and is 1.778 mm in an embodiment. First portion is continually and linearly connected to a second portion 1104 having a second length L2 ranging from 25.4 mm to 127 mm, and is 26.416 mm in an embodiment, and having a second width W2 ranging from 2.54 mm to 38.1 mm, which is 3.81 mm in an embodiment. Therefore, a total length of the electrode configuration ranges from 50.8 mm to 3.937 meters, and is 65.024 mm in an embodiment. Outer surface of first portion 1102 is insulated, whereas a portion of outer surface of second portion 1104, which is proximal to first portion 1102 is insulated, while the remaining surface is exposed.

FIG. 11B separately illustrates the different layers used to form the electrode configuration of FIG. 11A, in accordance with some embodiments of the present specification. Three layers form the configuration, comprising a middle layer 1106 which is between a top electrode layer 1108 and a bottom electrode layer 1110. Base material of each electrode layer is 34.79 μm cu/25 μm polyimide/34.79 μm cu. A cover layer covering both sides of the electrode layers is 12 μm polyimide, which extends up to 25 μm of both sides. Parts of the electrode layers are copper plated. Finished thickness of each layer is approximately 34.79 μm cu.

In one non-limiting example, a catheter having an inner balloon disposed within an outer balloon is evaluated to characterize temperature control and thermal dose delivery. The inner balloon and/or outer balloon is filled with a saline and contrast mixture (for example, a 50/50 saline-to-contrast mixture by volume) initially at approximately room temperature (about 25° C.). Thermal energy is delivered to the inner balloon fluid using a constant-power RF heating mode at a selected power level, such as about 30 W or about 50 W, for a treatment interval sufficient to reach a target interface condition.

During treatment, temperature is monitored at multiple locations, including (i) the inner balloon fluid temperature, (ii) the outer balloon fluid temperature, and (iii) a tissue-adjacent temperature measured using an embedded thermocouple located at a depth of about 1 mm from a tissue-contacting surface (or within a tissue phantom at an equivalent location). In some tests, a target tissue-interface temperature range is maintained for a selected duration to deliver a prescribed thermal dose. In tissue testing (e.g., ex vivo or in vivo), histology is obtained following treatment and demonstrates thermal injury characterized by tissue necrosis within the intima and superficial media, consistent with delivery of therapeutic thermal energy while limiting deeper wall injury.

Table 2 below illustrates results of different inflation protocols. The first column lists the different types of vessels and the second column lists the time of inflation.

TABLE 2
Inflation protocols for different vessels

| | Inflation | | Pressure | |
|---|---|---|---|---|
| Vessel | Time | | (atm) | Key Findings |
| Coronary | 30-60 | s | 8-12 | Extended inflations yield better stent |
| | | | | expansion but increase ischemia risk |
| Femoropopliteal | 180-300 | s | 6-10 | Longer durations reduce dissections |
| | | | | and stenting needs |
| Below-knee | 60-120 | s | 3-5 | Improves outcomes in chronic limb- |

| | | | |
|---|---|---|---|
| | | | threatening ischemia |
| Dialysis Access | 120 s + 60 s | 20-40 | Prolonged inflations maintain patency |
| | | | and reduce restenosis |

The following points provide exemplary inflation protocol considerations for different anatomical interventions:
1. For coronary interventions, 10-30 second inflation cycles are employed to balance plaque modification and ischemia prevention. Extended protocols such as those involving 380 second inflations, can achieve significant lumen gain, but risk myocardial ischemia.
2. For femoropopliteal interventions, prolonged inflations (>3 minutes) reduce flow-limiting dissections by 55%. CTOs longer than 150 mm benefit from 7-10 minute inflations, while eccentric plaques respond well to pulsed inflations.
3. For below-knee arteries, ACOART-BTK trial demonstrates better outcomes with 60 second base inflations followed by 180 second post-dilation, particularly for chronic limb-threatening ischemia.
4. For dialysis access maintenance, AV fistulas often require baseline inflations of 60-120 seconds at 20 atm, with stepwise pressure increments to 40 atm for cases with persistent stenosis.

The observations of Table 1 and the point listed above, highlight the importance of inflation duration and pressure parameters in optimizing plaque modification, lumen gain, and procedural success across various vascular territories.

An IVL procedure uses acoustic pressure waves (also known as sonic pressure waves) to break plaque deposits in arteries. The procedure involves a catheter with emitters that deliver acoustic pressure waves proximate the calcified plaque within an artery. An ultrasound core is positioned around a guidewire. An inflatable balloon is positioned at a distal end of the catheter. A portable energy generator sources energy to operate the two sets of radiopaque and traditional emitters, which are within the central and lateral boundaries of the balloon. The emitters produce pulsed acoustic pressure waves. In a typical cycle, 10 pulses of acoustic pressure waves are delivered. The acoustic pressure waves are delivered in the form of mechanical energy to the target region comprising calcified plaque. A sequence of pulses result in creating cracks that assist break-down of the calcified plaque deposits. Each cycle of treatment may contain a specific number of pulses. Furthermore, between each cycle, the ballon is deflated to allow formed bubbles resulting from cracked plaque to disburse safely, and then the procedure is repeated. The following paragraphs provide an overview of the impact of different acoustic pressure cycles.

IVL cycles have hemodynamic and ischemic consequences. 10-second cycles in coronary arteries induce 40-50% ATP depletion and myocardial pH decline to 6.8 within 15 seconds of occlusion. 30-second cycles in peripheral vessels result in skeletal muscle lactate accumulation (8.1 mmol/L vs. 4.2 mmol/L for 10 s cycles) and increased ischemic burden (28% higher).

IVL procedure has potential consequences of arrhythmia. Ventricular capture incidence increases from 12% with 10-second cycles to 41% with 30-second cycles. Sustained VT/VF incidence rises to 1.9% with longer cycles, and atrial fibrillation increases from 0.3% to 3.8%. Pre-treatment with ivabradine reduces capture risk by 57%. An IVL procedure additionally affects calcium modification efficacy. 30-second cycles improve calcium fracture incidence (67.4% vs. 42.9%) and increase mean fracture width (1.32 mm vs. 0.55 mm). However, balloon rupture risk triples in tibial arteries (12% vs. 4% for 10-second cycles).

Clinically, 30-day major adverse cardiac events (MACE) rates are higher with 30-second IVL cycles (5.7% mortality, 9.4% peri-procedural MI) compared to 10-second cycles (0.5% mortality, 5.8% peri-procedural MI). Whereas, hybrid protocols involving 10 s inflation followed by 5 s deflation, and then repeat, reduce MACE by 38% while preserving efficacy.

Iliac arteries require 30-second cycles for optimal calcium modification. Femoropopliteal lesions benefit from 20-second cycles, improving acute gain by 23%. Coronary arteries typically maintain 10-second cycles to avoid hemodynamic collapse.

Pressure-regulated IVL systems with MEMS sensors reduce ischemic burden by 41%, thus optimizing an IVL procedure. Oxygen-eluting balloons maintain tissue pO2>15 mmHg during extended inflations, which also assists in the optimization of the IVL procedure. Further, adaptive pulse sequencing reduces pulse requirements by 29% using real-time calcium density analysis. The above paragraphs demonstrate the differential effects of inflation cycle duration across vascular territories and underscore the importance of cycle selection for optimizing efficacy and safety.

In one non-limiting example, lithotripsy performance is evaluated using computer simulation to quantify a performance metric referred to as pulse efficiency. In this example, pulse efficiency is defined using one or more objective outcomes, including peak pressure at or near the balloon wall (e.g., a simulated pressure waveform metric) and a fracture outcome metric (e.g., predicted fracture count and/or fracture incidence in a calcified target model). Simulations are performed across multiple runs (for example, n=5) under otherwise similar conditions while varying a residual fluid volume (or residual layer thickness) between the inner and outer balloons during pulse delivery.

In one simulation set, reducing the residual coupling volume between the inner balloon and the outer balloon during pulse delivery increases peak pressure at the balloon wall and increases predicted fracture count relative to a larger residual-volume condition. In some embodiments, the residual coupling volume is reduced by controlling the inflation state of one or both balloons such that the coupling layer between the balloons is thin (e.g., less than about 1 mm) during pulse delivery. In some embodiments, the magnitude of pulse efficiency improvement depends on the particular balloon geometry and the residual coupling volume associated with that geometry. For example, when the residual coupling volume corresponds to a first balloon size and fluid volume configuration (for example, a smaller-diameter balloon configuration having an associated inner balloon volume as described elsewhere herein), the simulation indicates an improvement in pulse efficiency on the order of about 63% relative to a larger residual-volume baseline. When the residual coupling volume corresponds to a second balloon size and fluid volume configuration (for example, a larger-diameter balloon configuration having an associated inner balloon volume as described elsewhere herein), the simulation indicates an improvement in pulse efficiency on the order of about 74% relative to a larger residual-volume baseline. These simulation results support that controlling inner balloon volume and outer balloon pressure/volume to reduce residual coupling volume during pulse delivery can improve lithotripsy coupling and efficiency, while still permitting the system to transition to thermal delivery and/or insulation states as described elsewhere.

By using the fluid volume between the inner and outer balloon to deliver the IVL pulse, the efficiency is significantly improved for any given outer balloon size. This phenomenon can be understood with an experiment: a double-balloon structure system with an outer balloon diameter of 4 mm, 6 mm, 8 mm, and 10 mm, each 25 mm in length were used with the embodiments of the present specification. The inner balloon diameter was 1 mm smaller than the outer balloon diameter, with length of the inner balloon shorter by 2 mm than the length of the outer balloon. The residual fluid volume was obtained by subtracting the inner balloon volume from the outer balloon volume, where the volume was calculated using the formula for the volume of a cylinder: V=πr²h, where r is the radius (diameter/2) and h is the length. The catheter device of the present specification was operated for each of the different balloon sizes. Acoustic pressure wave attenuation and required pulse increase for the outer balloon were measured as baseline data. Adjusted values were calculated based on the ratio of the residual fluid volume to the outer balloon volume, assuming attenuation and pulse requirements decrease proportionally as residual volume decreases. The results from the experiment are illustrated in Table 3 below.

TABLE 3
Results from the experiment

| | | | | Pressure Attenuation | | Pulse Increase | |
|---|---|---|---|---|---|---|---|
| Diameter | Volume (ml) | | | Outer | Residual | Outer | Residual |
| (mm) | Outer | Inner | Residual | % | % | % | % |
| 4 | 0.31 | 0.16 | 0.15 | 12 | 5.81 | 0 | 0.00 |
| 6 | 0.71 | 0.45 | 0.26 | 29 | 10.62 | 22 | 8.06 |
| 8 | 1.26 | 0.89 | 0.37 | 47 | 13.80 | 41 | 12.04 |
| 10 | 1.96 | 1.46 | 0.50 | 63 | 16.07 | 67 | 17.09 |

Therefore, it was observed that the space between the inner and outer balloons retains a reduced residual fluid volume that significantly affects and improves pulse efficiency. With the residual volume present, the acoustic pressure wave attenuation decreased by 51% for the 4 mm balloon and by about 75% for the 10 mm balloon compared to their values using only an outer balloon. Also, the required pulse increase was reduced by 63% for the 6 mm balloon and 74% for the 10 mm balloon, indicating substantial efficiency gains when using the coaxial balloon configuration. Further, incorporating an inner balloon enhances pulse efficiency, reducing energy requirements for the same therapeutic effect while maintaining structural integrity.

It can be concluded that the coaxial balloon embodiments of the present specification introduce multiple benefits including those in improving pulse delivery efficiency through strategic fluid volume reduction.

Shorter IVL cycle durations are adopted by the embodiments of the present specification. The shorter durations provide an enhanced acoustic efficiency, since the coaxial double-balloon configuration maximizes energy delivery and minimizes wave attenuation. Efficient energy transfer allows for comparable plaque modification with shorter inflation durations.

Additionally, shorter IVL cycle durations reduce ischemic risks currently associated with IVL therapy. The ischemic complications are minimized due to decrease in myocardial oxygen debt resulting from shorter cycle times. In coronary vessels, 10 second cycles maintain 42% collateral flow, thereby reducing ischemic events.

The clinical outcomes, as observed through the experiment, were optimized due to reduced IVL cycle durations. Hybrid protocols that simultaneously deliver thermal energy with acoustic pressure waves, with 10 second inflation and 5 second deflation cycles, reduced MACE incidence by 38%. Additionally, improved acoustic targeting decreased procedural times without compromising calcium fracture efficacy.

Furthermore, the acoustic wave propagation generated by the pulses is optimized through precise emitter positioning and dynamic frequency adjustments. Also, the hybrid approach of the embodiments of the present specification demonstrated, through experiments, improved luminal gain and reduced restenosis rates with this hybrid approach, compared to traditional catheter devices such as balloon angioplasty, cryoplasty, and IVL-only catheters.

Advantages of the embodiments of the present specification include achievement of comprehensive plaque modification from simultaneous application of thermal energy and acoustic energy and through dual-modality interaction. Elastic recoil is reduced due to thermal remodeling. Lower restenosis rates are achieved through apoptosis induction in hyperplastic neointimal tissue. The procedural workflow is simplified compared to systems and processes that require use of separate devices. The luminal gain is enhanced and vessel compliance is improved. Procedural efficiency is also increased with reduced treatment time.

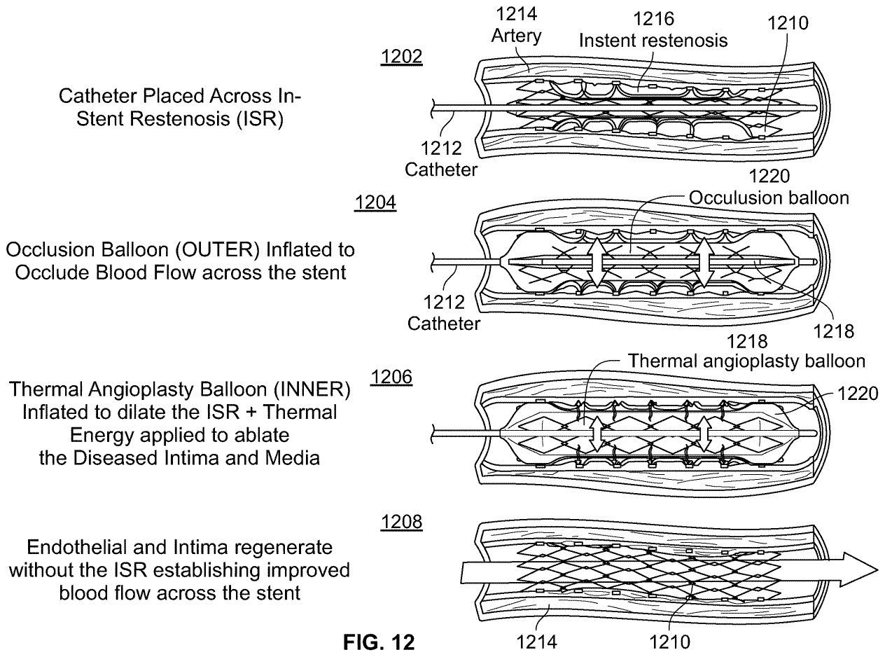

FIG. 12 illustrates an exemplary sequence of steps of thermal balloon angioplasty while a catheter is positioned within an artery 1214 with a stent 1210 and ISR 1216, in accordance with some embodiments of the present specification. At step 1202, a catheter 1212 is placed inside an artery 1214 with ISR 1216. The catheter 1212 is positioned so that distal end of catheter 1212 is placed across the length of ISR 1216. The distal end of catheter 1212 comprises a thermal angioplasty balloon 1218 configured within an outer occlusion balloon 1220. At step 1204, outer occlusion balloon 1220, which is initially in a compressed state, is inflated with saline/CO₂ or contrast to occlude blood flow across the length of ISR 1216. At step 1206, thermal angioplasty balloon (inner balloon) 1218 is inflated to dilate ISR 1216. Additionally, ablative fluid is heated by the electrode within the catheter, to generate thermal energy in the chamber within balloon 1218. Ablative fluid occupies balloon 1218, and at hot zones where inner balloon 1218 touches outer balloon 1220, the diseased intima and media is ablated. The catheter 1212 is withdrawn and removed from the target site within artery 1214 after the ablative procedure is completed. At step 1208, over a period of time, endothelial lining and intima regenerate at the target site within artery 1214, without ISR 1216, thereby establishing improved blood flow across stent 1210.

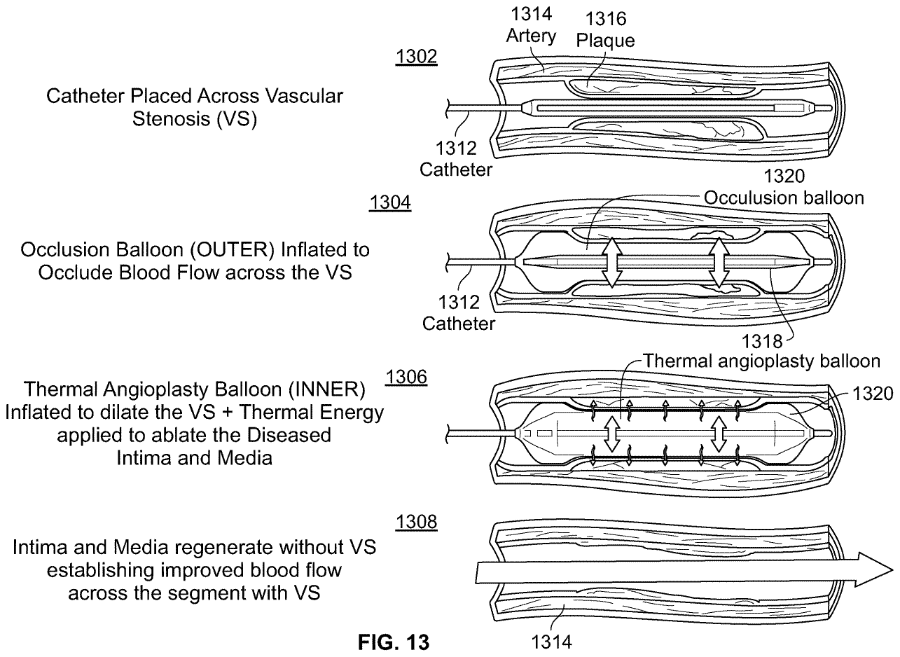

FIG. 13 illustrates an exemplary sequence of steps of thermal balloon angioplasty while a catheter is positioned within an artery 1314 with vascular stenosis (VS), in accordance with some embodiments of the present specification. At step 1302, a catheter 1312 is placed inside artery 1314 with VS comprising plaque 1316 at the target site. Catheter 1312 is positioned so that distal end of catheter 1312 is placed across the length of plaque 1316. The distal end of catheter 1312 comprises a thermal angioplasty balloon 1318 configured within an outer occlusion balloon 1320. At step 1304, outer occlusion balloon 1320, which is initially in a compressed state, is inflated with CO₂, saline or contrast to occlude blood flow across the length of plaque 1316. At step 1306, thermal angioplasty balloon (inner balloon) 1318 is inflated to dilate plaque 1316. Additionally, ablative fluid is heated by the electrode within the catheter, to generate thermal energy within balloon 1318. Ablative fluid occupies balloon 1318, and at hot zones where balloon 1318 touches outer balloon 1320, the diseased intima and media is ablated. Catheter 1312 is withdrawn and removed from the target site within artery 1314 after the ablative procedure is completed. At step 1308, over a period of time, endothelial lining and intima regenerate at the target site within artery 1314, without plaque 1316, thereby establishing improved blood flow within the arterial segment with VS.

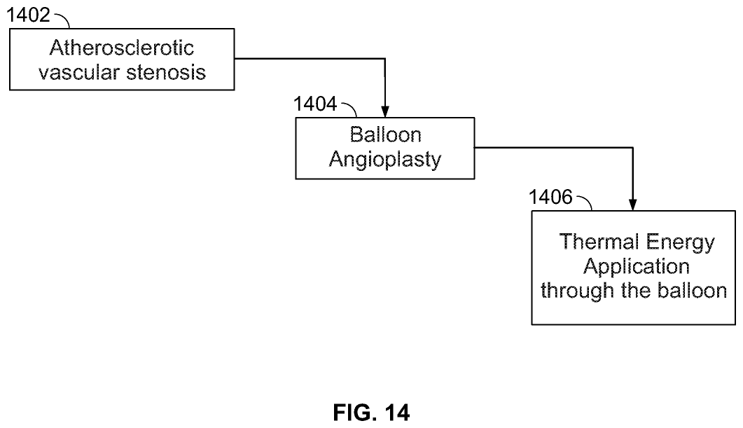

FIG. 14 is a flow chart illustrating the fundamental sequence of steps to achieve thermal angioplasty to treat atherosclerotic VS, in accordance with an embodiment of the present specification. At step 1402, the phenomena and location of atherosclerotic VS is identified. At step 1404, a distal end of a catheter comprising a balloon is positioned proximate the atherosclerotic VS and the balloon is inflated to perform balloon angioplasty. At step 1406, thermal ablative energy is applied through the balloon to ablate the diseased portions at the target site.

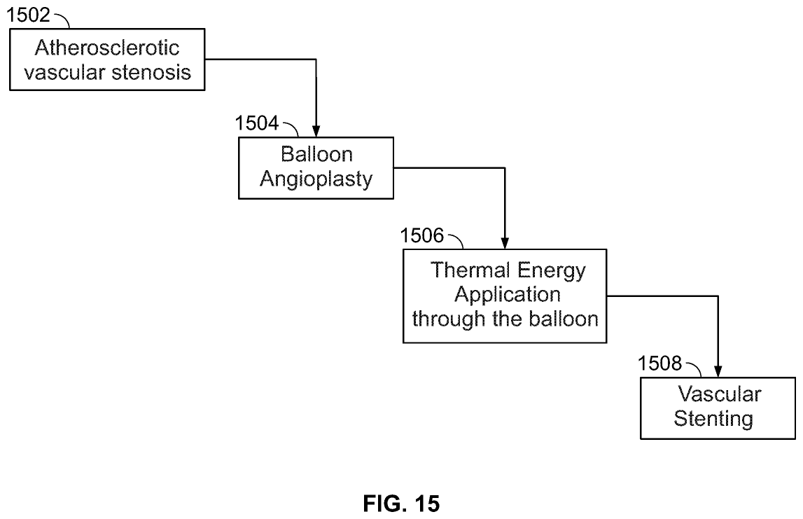

FIG. 15 is a flow chart illustrating another exemplary sequence of steps to achieve thermal angioplasty to treat atherosclerotic VS, in accordance with an embodiment of the present specification. At step 1502, the phenomena and location of atherosclerotic VS is identified. At step 1504, a distal end of a catheter comprising a balloon is positioned proximate the atherosclerotic VS and the balloon is inflated to perform balloon angioplasty. At step 1506, thermal ablative energy is applied through the balloon to ablate the diseased portions at the target site. At step 1508, a stent is positioned within the vasculature at the target site.

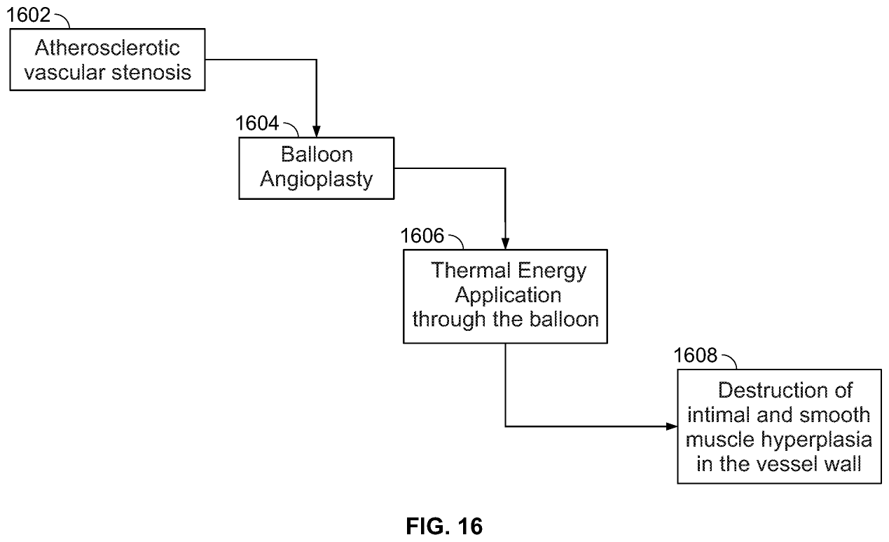

FIG. 16 is a flow chart illustrating another exemplary sequence of steps to achieve thermal angioplasty to treat atherosclerotic VS, in accordance with an embodiment of the present specification. At step 1602, the phenomena and location of atherosclerotic VS is identified. At step 1604, a distal end of a catheter comprising a balloon is positioned proximate the atherosclerotic VS and the balloon is inflated to perform balloon angioplasty. At step 1606, thermal ablative energy is applied through the balloon to ablate the diseased portions at the target site. At step 1608, as a result of the ablation procedure, intimal and smooth muscle hyperplasia in the vessel wall at the target site are destroyed.

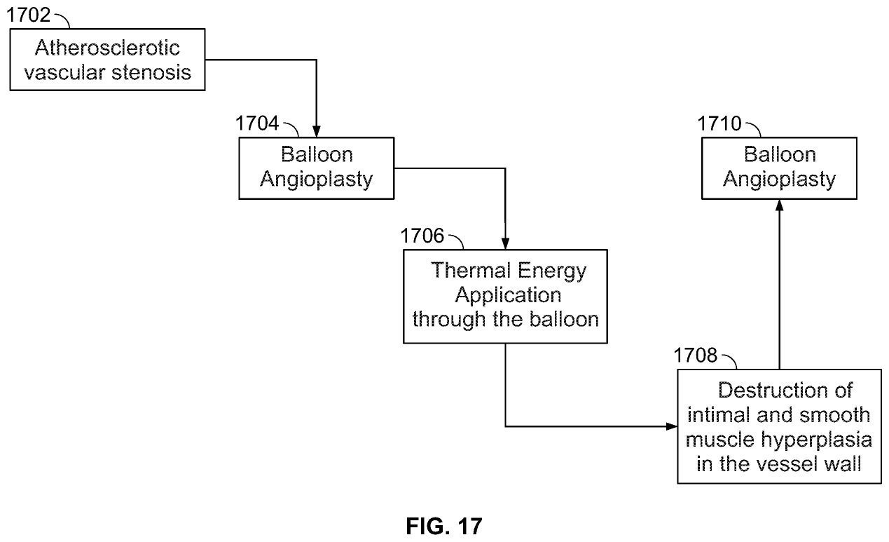

FIG. 17 is a flow chart illustrating another exemplary sequence of steps to achieve thermal angioplasty to treat atherosclerotic VS, in accordance with an embodiment of the present specification. At step 1702, the phenomena and location of atherosclerotic VS is identified. At step 1704, a distal end of a catheter comprising a balloon is positioned proximate the atherosclerotic VS and the balloon is inflated to perform balloon angioplasty. At step 1706, thermal ablative energy is applied through the balloon to ablate the diseased portions at the target site. At step 1708, as a result of the ablation procedure, intimal and smooth muscle hyperplasia in the vessel wall at the target site are destroyed. Subsequently, at step 1710, the method of step 1704 is repeated.

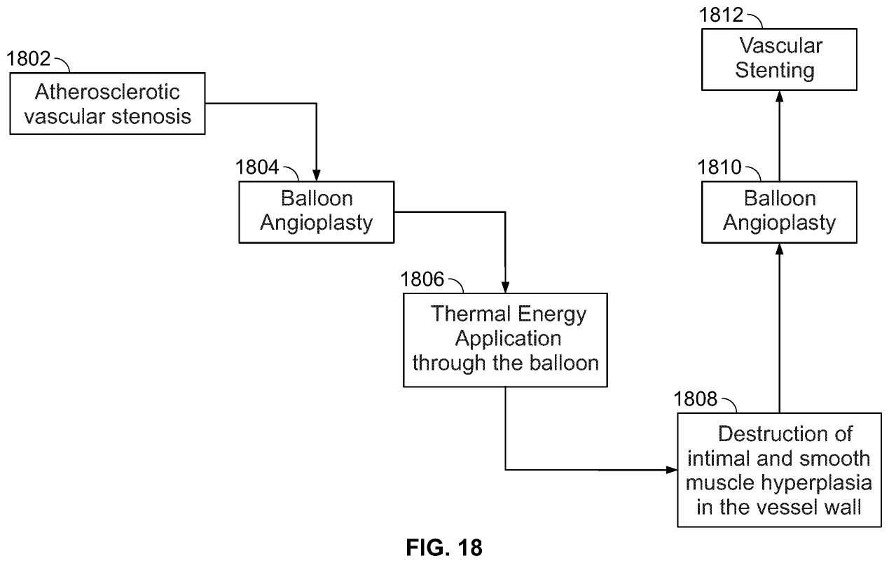

FIG. 18 is a flow chart illustrating another exemplary sequence of steps to achieve thermal angioplasty to treat atherosclerotic VS, in accordance with an embodiment of the present specification. At step 1802, the phenomena and location of atherosclerotic VS is identified. At step 1804, a distal end of a catheter comprising a balloon is positioned proximate the atherosclerotic VS and the balloon is inflated to perform balloon angioplasty. At step 1806, thermal ablative energy is applied through the balloon to ablate the diseased portions at the target site. At step 1808, as a result of the ablation procedure, intimal and smooth muscle hyperplasia in the vessel wall at the target site are destroyed. Subsequently, at step 1810, the method of step 1804 is repeated. At step 1812, a stent is positioned within the vasculature at the target site.

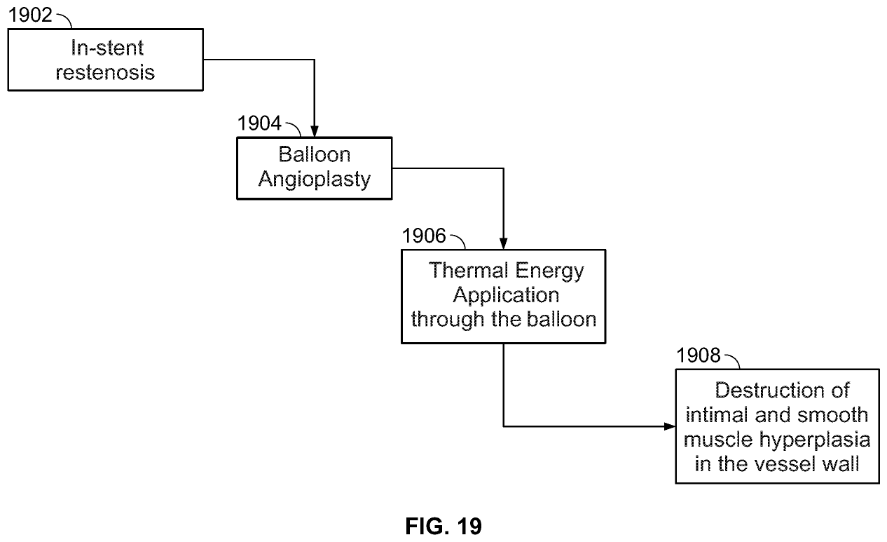

FIG. 19 is a flow chart illustrating an exemplary sequence of steps to achieve thermal angioplasty to treat ISR, in accordance with an embodiment of the present specification. At step 1902, the phenomena and location of ISR is identified. At step 1904, a distal end of a catheter comprising a balloon is positioned proximate the atherosclerotic VS and the balloon is inflated to perform balloon angioplasty. At step 1906, thermal ablative energy is applied through the balloon to ablate the diseased portions at the target site. At step 1908, as a result of the ablation procedure, intimal and smooth muscle hyperplasia in the vessel wall at the target site are destroyed.

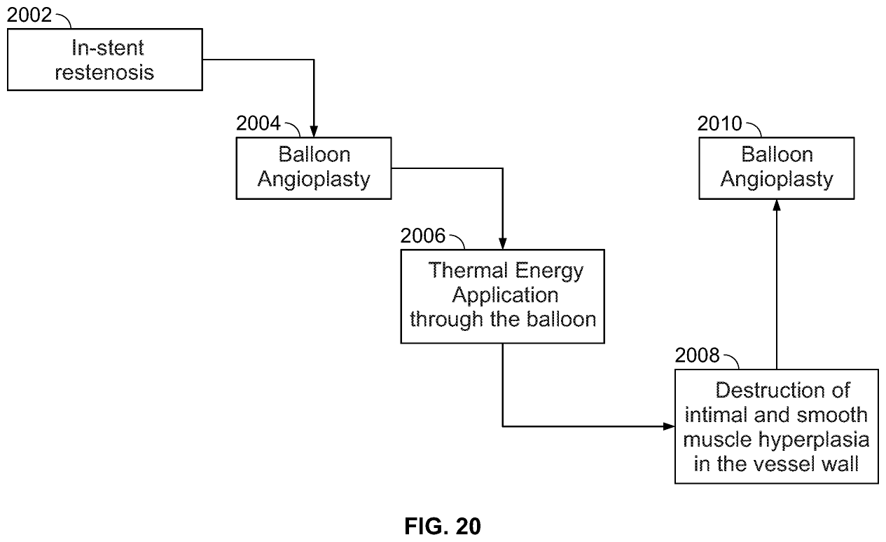

FIG. 20 is a flow chart illustrating another exemplary sequence of steps to achieve thermal angioplasty to treat ISR, in accordance with an embodiment of the present specification. At step 2002, the phenomena and location of ISR is identified. At step 2004, a distal end of a catheter comprising a balloon is positioned proximate the atherosclerotic VS and the balloon is inflated to perform balloon angioplasty. At step 2006, thermal ablative energy is applied through the balloon to ablate the diseased portions at the target site. At step 2008, as a result of the ablation procedure, intimal and smooth muscle hyperplasia in the vessel wall at the target site are destroyed. Subsequently, at step 2010, step 2004 is repeated.

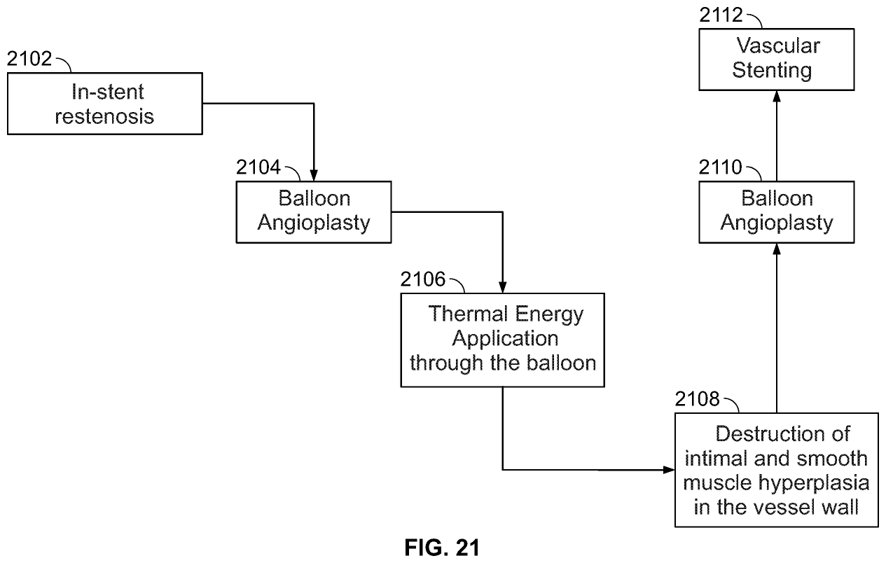

FIG. 21 is a flow chart illustrating another exemplary sequence of steps to achieve thermal angioplasty to treat ISR, in accordance with an embodiment of the present specification. At step 2102, the phenomena and location of ISR is identified. At step 2104, a distal end of a catheter comprising a balloon is positioned proximate the atherosclerotic VS and the balloon is inflated to perform balloon angioplasty. At step 2106, thermal ablative energy is applied through the balloon to ablate the diseased portions at the target site. At step 2108, as a result of the ablation procedure, intimal and smooth muscle hyperplasia in the vessel wall at the target site are destroyed. Subsequently, at step 2110, step 2104 is repeated. At step 2112, a stent is positioned within the vasculature at the target site.

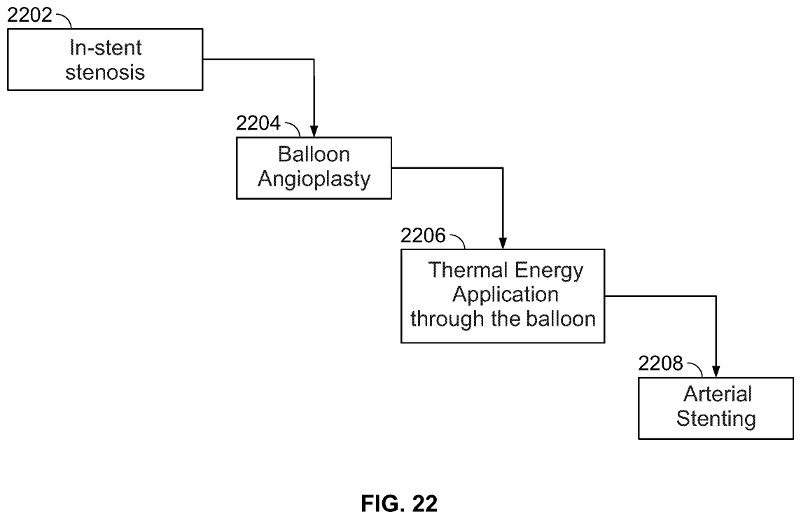

FIG. 22 is a flow chart illustrating another exemplary sequence of steps to achieve thermal angioplasty to treat ISR, in accordance with an embodiment of the present specification. At step 2202, the phenomena and location of ISR is identified. At step 2204, a distal end of a catheter comprising a balloon is positioned proximate the atherosclerotic VS and the balloon is inflated to perform balloon angioplasty. At step 2206, thermal ablative energy is applied through the balloon to ablate the diseased portions at the target site. At step 2208, a stent is positioned within the artery at the target site.

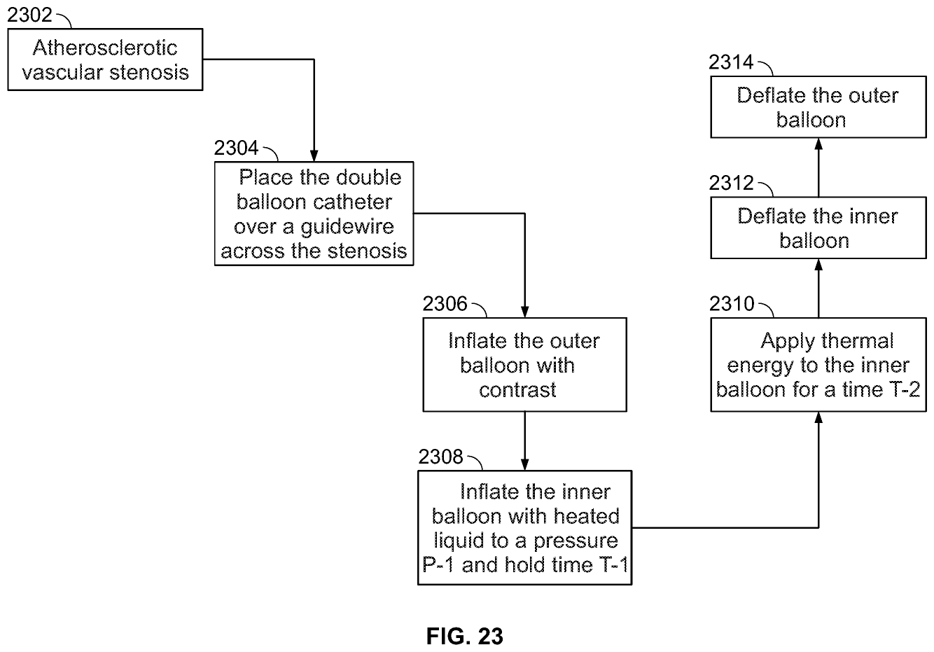

FIG. 23 is a flow chart illustrating another exemplary sequence of steps of thermal ablation to treat atherosclerotic VS, in accordance with an embodiment of the present specification. At step 2302, the phenomena and location of atherosclerotic VS is identified. At step 2304, a distal end of a catheter comprising inner and outer balloon configurations, are positioned over a guidewire at a location proximate the atherosclerotic VS (target site). At step 2306, the outer balloon is inflated with contrast. At step 2308, the inner balloon is inflated with saline to a pressure P1 and for a time period of T1. At step 2310, thermal ablative energy is applied through the inner balloon to ablate the diseased portions at the target site, for a time period T2. At step 2312, the inner balloon is deflated. Ats step 2314, the outer balloon is deflated.

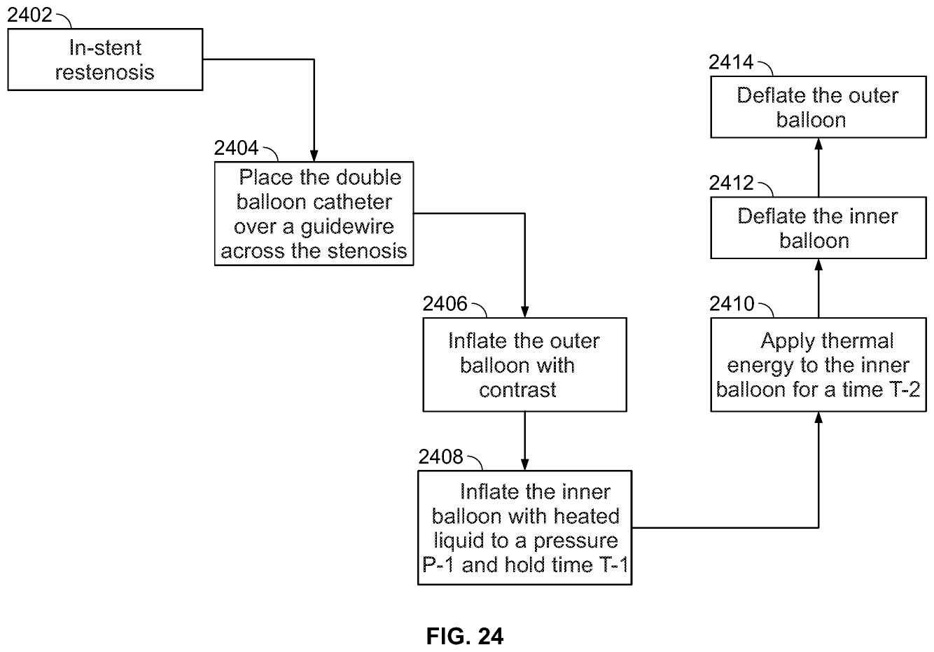

FIG. 24 is a flow chart illustrating another exemplary sequence of steps of thermal ablation to treat ISR, in accordance with an embodiment of the present specification. At step 2402, the phenomena and location of ISR is identified. At step 2404, a distal end of a catheter comprising inner and outer balloon configurations, are positioned over a guidewire at a location proximate the ISR (target site). At step 2406, the outer balloon is inflated with contrast. At step 2408, the inner balloon is inflated with saline to a pressure P1 and for a time period of T1. At step 2410, thermal ablative energy is applied through the inner balloon to ablate the diseased portions at the target site, for a time period T2. At step 2412, the inner balloon is deflated. At step 2414, the outer balloon is deflated.

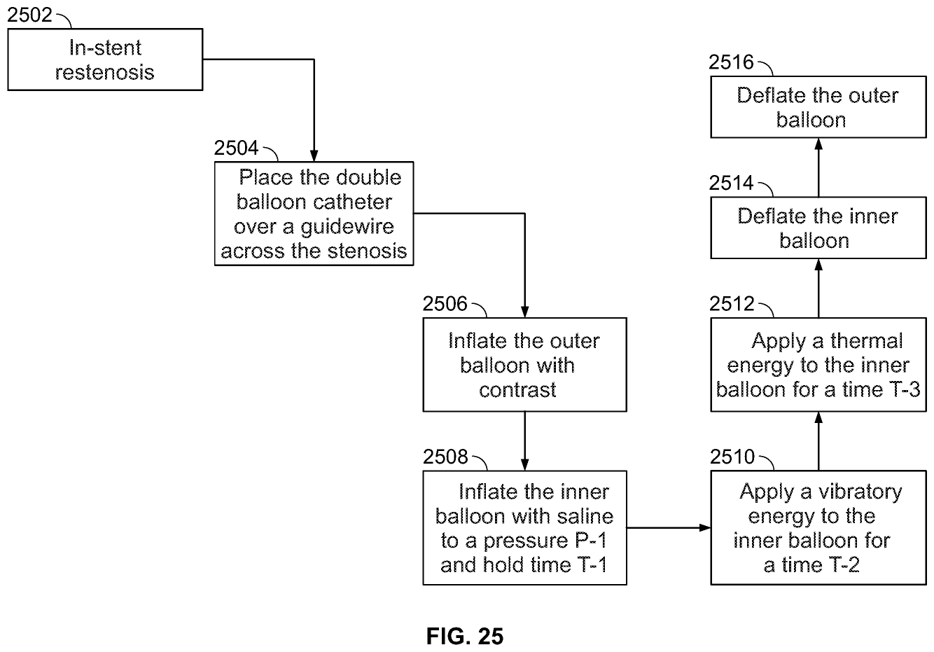

FIG. 25 is a flow chart illustrating another exemplary sequence of steps of thermal ablation to treat ISR, in accordance with an embodiment of the present specification. At step 2502, the phenomena and location of ISR is identified. At step 2504, a distal end of a catheter comprising inner and outer balloon configurations, are positioned over a guidewire at a location proximate to the ISR (target site). At step 2506, the outer balloon is inflated with contrast. At step 2508, the inner balloon is inflated with saline to a pressure P1 and for a time period of T1. At step 2510, a vibratory energy is applied through the inner balloon, for a time period T2, during the time T1 when the inner balloon is inflated with saline to pressure P1. At step 2512, thermal ablative energy is applied through the inner balloon to ablate the diseased portions at the target site, for a time period T3, also during the time T1 when the inner balloon is inflated with saline to pressure P1. At step 2514, the inner balloon is deflated. Ats step 2516, the outer balloon is deflated.

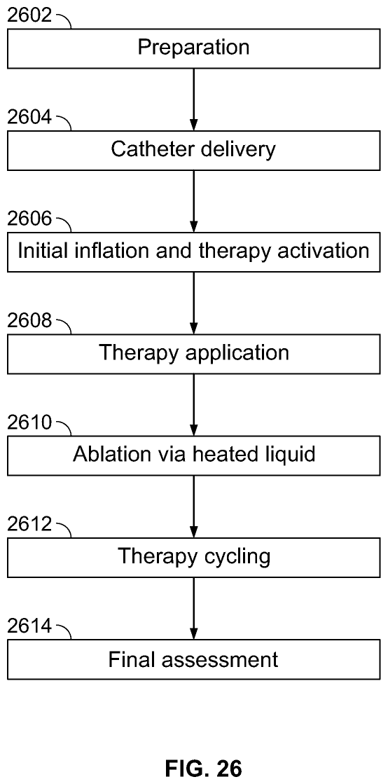

FIG. 26 is a flow chart that illustrates an exemplary process of an integrated TBA and IVL procedure using a dual mode catheter, in accordance with some embodiments of the present specification. At step 2602, a site for insertion of the dual-mode catheter is identified and prepared. The site can be an arterial or a vascular lumen with at least one target lesion that may comprise calcified plaque deposits. A guidewire is introduced through an access point to the vascular lumen and placed across the target lesion. A guide sheath is passed using the guidewire and which is positioned at a location proximate the target lumen. At step 2604, a catheter is selected of a size that is appropriate for the target lesion characteristics. The selected catheter is then advanced over the guidewire through the guide sheath to the target lesion site. The position of the catheter is confirmed using methods such as fluoroscopy and anatomical landmarks.

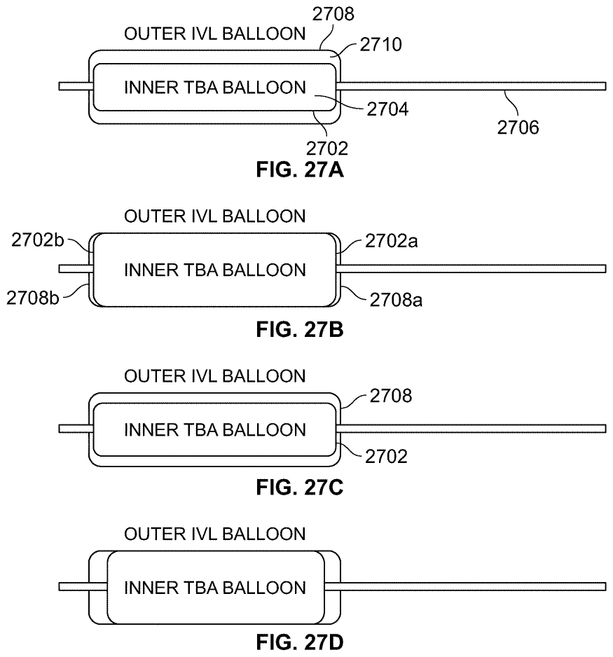

Referring again to FIG. 26, at step 2606, the inner balloon is inflated with saline to a volume that builds a pressure of 2 atm to 4 atm within the inner balloon. Additionally, the outer balloon is inflated with a 50/50 saline-contrast mix to 2.1 atm to 5 atm, ensuring a pressure of at least 0.1 atm higher than that of the inner balloon. FIG. 27A illustrates an inner balloon 2702 filled with saline 2704 positioned coaxially around a guide sheath 2706 within an outer balloon 2708 filled with 50/50 saline contrast mix 2710, in accordance with some embodiments of the present specification.

At step 2608, the integrated therapy application is initiated. Initially, RF electrodes are activated to initiate heating of the saline within the inner balloon to reach a temperature of 60° C. to 65° C. within a duration of 5 to 10 seconds. The delivery of thermal energy is maintained for 30-60 s. Precision feedback control mechanism in the catheter configuration ensure that the temperature is maintained. The IVL therapy is also activated to deliver pulses at a frequency of 1 Hz.

At step 2610, outer balloon 2708 is deflated to a level of 2-4 atm to allow thermal contact of the heated inner balloon 2702 with the target lesion through outer balloon 2708. The thermal therapy is maintained at a temperature range of 60-65° C. for 10-60 s while the IVL therapy is continued with pulses delivered at a frequency of 1 Hz. FIG. 27B illustrates heated inner balloon 2702 which, in its expanded (inflated) form, contact outer balloon 2708 to create a hot ablation zone, in accordance with some embodiments. The dimensions of inner balloon 2702 and outer balloon 2708 are designed such that when inner balloon 2702 is inflated, a proximal end 2702a and a distal end 2702b of inner balloon 2702 do not contact respective proximal end 2708a and distal end 2708b of outer balloon 2708, thereby creating a cooler non-ablative zone.

At step 2612, outer balloon 2708 is re-inflated by re-introducing the 50/50 saline-contrast mixture to form an insulating layer. Heated inner balloon 2702 does not contact outer balloon 2708 so that the entire surface of outer balloon 2708 returns to cooler non-ablative temperatures. FIG. 27C illustrates a gap arising due to non-contact between surfaces of inner balloon 2702 and outer balloon 2708, in accordance with some embodiments of the present specification. The acoustic pressure pulses are applied to the fluid comprising 50/50 saline-contrast mixture between inner balloon 2702 and outer balloon 2708. Additionally, thermal therapy is repeated by heating the RF electrodes that result in heating of the saline within inner balloon 2702. In this manner, thermal and IVL cycles are repeated until adequate dilation of the target lesion is achieved. FIG. 27D illustrates a hybrid mode achieved by alternatively cycling between the thermal ablation mode and the IVL mode or by applying IVL simultaneously with thermal energy, in accordance with some embodiments of the present specification. In the hybrid mode, inner 2702 and outer 2708 balloons are in contact along their elongated cylindrical surfaces, to create an ablation zone parallel to the axis of inner balloon 2702 and outer balloon 2708, while proximal 2702a, 2708a and distal 2702b, 2708b ends are not in contact thereby creating a non-ablation zone, while acoustic pressure pulses are being simultaneously delivered to residual fluid in outer balloon 2708.

At step 2614, inner balloon 2702 and outer balloon 2708 are deflated. The catheter is then withdrawn and removed from the target site. Additionally, angiographic imaging is conducted to assess the degree of modification in the plaque at the target site, and vessel patency.

Systems and methods of the present specification utilize relatively smaller fluid volumes in the outer balloon, as compared to existing IVL methods, which enhances energy transfer from the acoustic pressure pulses by reducing dispersion and optimizing focus of the pulses. Smaller fluid volume reduces acoustic wave attenuation, enhances wave focus on calcified plaques, and ensures more uniform energy distribution. Herein, the term waves refer to the emission of acoustic pulses at a specific frequency. Larger fluid volumes require additional transducers and result in inefficient energy transfer due to dispersion and refraction. Optimal fluid volume depends on balloon diameter and vessel characteristics, with smaller balloons benefiting from more concentrated energy delivery.

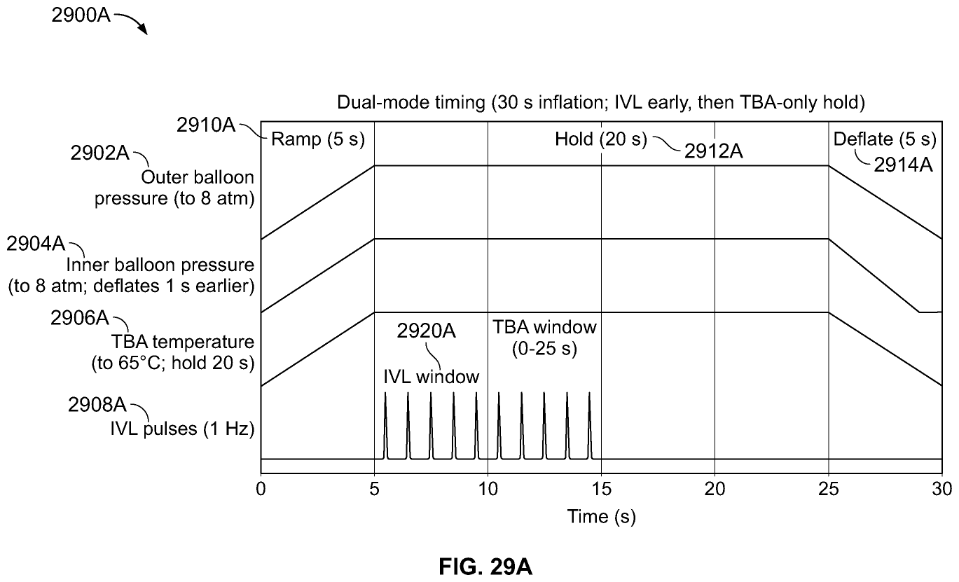

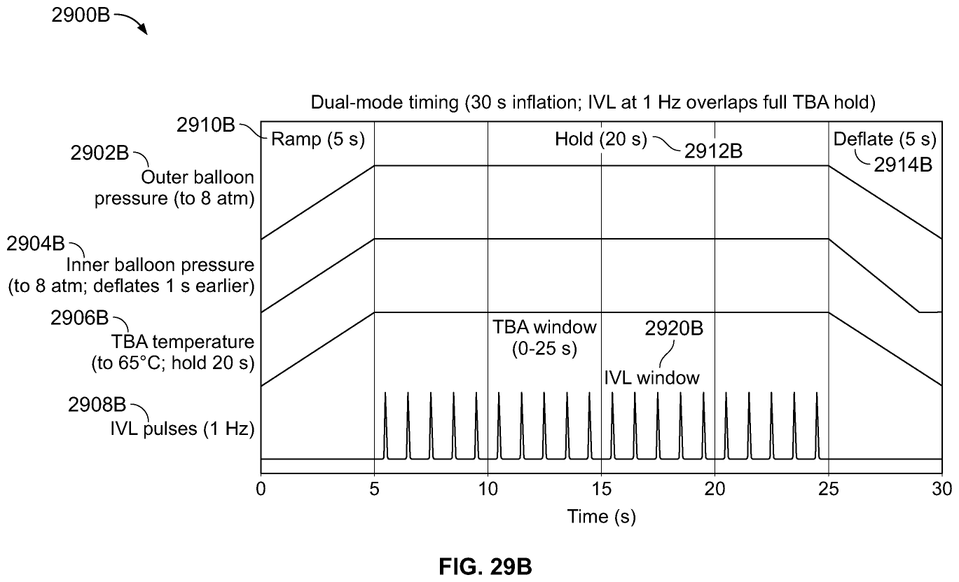

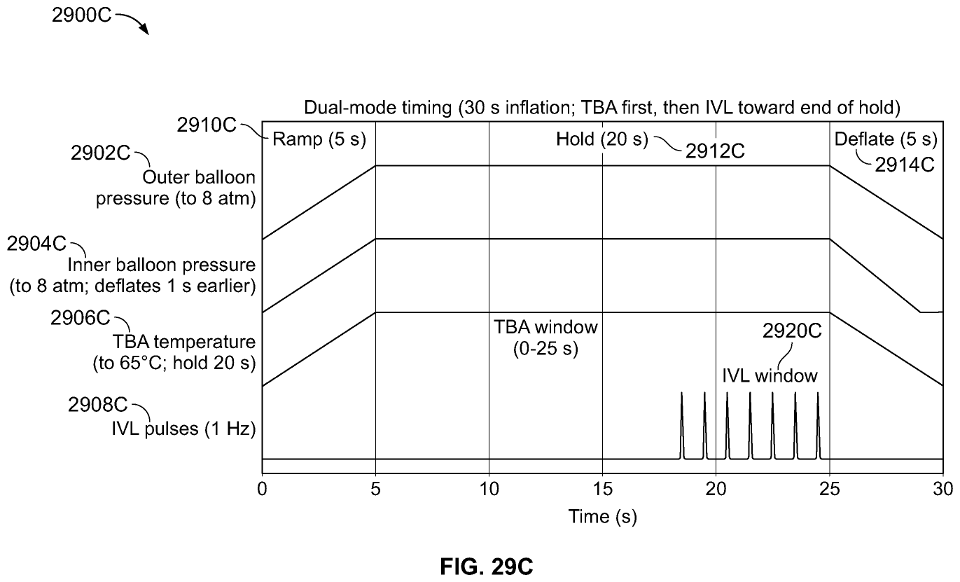

Referring to FIGS. 29A to 29C, another administration protocol for the concurrent delivery of thermal ablative energy and acoustic pressure waves is shown using coordinated control of an outer balloon and an inner balloon. In each illustrated variant, there is a first ramping up phase 2910A, 2910B, 2910C, a second therapy phase 2912A, 2912B, 2912C, and a third ramping down phase 2914A, 2914B, 2914C. In the first phase, the controller executes an inflation cycle that is preferably in a range of 10 seconds to 120 seconds. In one embodiment, the first phase is approximately 30 seconds and includes a first interval (in a range of 2 to 10 seconds, preferably 5 seconds) in which one or both balloons are inflated toward a maximum pressure (e.g., about 2 to 100 atm, preferably 6 to 10 atm, even more preferably 8 atm) while the thermal system ramps toward a maximum commanded temperature (e.g., about 55° C. to 75° C., preferably 60-70° C., more preferably 65° C.). The second phase is in a range of 10 to 25 seconds (preferably 20 seconds) in which inflation pressure and temperature are maintained relatively constant (e.g., about 8 atm and about 65° C.), and the third phase is in a range of 2 to 10 seconds (preferably 5 seconds) in which the balloons are deflated, with the inner balloon completing deflation earlier than the outer balloon (e.g., by about 0.2 to 5 seconds, preferably 1 second). The behavior of the outer balloon, inner balloon, and temperature through each phase is shown in 2902A, B, C, 2904A, B, C, 2906A, B, C, respectively.

In the depicted embodiments, IVL pulses, or acoustic waves, are delivered during the second phase. Referring to FIG. 29A, acoustic pulse delivery 2920A is performed during an initial portion of the second phase treatment window, after which the system continues thermal energy delivery without delivering IVL acoustic pulse waves for the remainder of the second phase. For example, IVL pulses may be delivered during the first 30-50% of the second phase time period while the thermal system is maintained at the commanded temperature. This sequencing allows early plaque modification by IVL, followed by continued thermal delivery during the remaining hold time.

Referring to FIG. 29B, acoustic pulse delivery 2920B is performed largely throughout the second phase treatment window. For example, IVL pulses may be delivered during 80-100% of the second phase time period while the thermal system is maintained at the commanded temperature. The controller adjusts heater power (including duty cycle) and balloon pressure/volume during overlap to maintain thermal and pressure safety limits while achieving a prescribed IVL pulse count and a prescribed thermal endpoint.

Referring to FIG. 29C, acoustic pulse delivery 2920C is performed during an end portion of the second phase treatment window, before which the system continues thermal energy delivery without delivering IVL acoustic pulse waves for the beginning of the second phase. For example, IVL pulses may be delivered during the last 30-50% of the second phase time period while the thermal system is maintained at the commanded temperature.

As discussed above, the controller may select timing boundaries, pulse counts, and transitions based on measured variables including inner and outer balloon pressures, measured temperatures (e.g., inner balloon fluid temperature, outer balloon fluid temperature, and/or a tissue-adjacent temperature), contact state and/or coupling-layer thickness, estimated thermal dose accumulation, and procedural constraints including allowable occlusion time. In some embodiments, the controller adaptively selects among the sequencing variants during a procedure to achieve acoustic and thermal objectives while reducing ischemic burden and maintaining safety limits.

In various embodiments, the controller is configured to select, adjust, and/or switch the outer balloon medium and/or one or more properties of the outer balloon medium, such as concentration, temperature, flow rate, or pressure, based on one or more procedural and/or anatomical parameters. For example, the controller may select a first outer balloon medium for coronary vasculature and a second outer balloon medium for peripheral vasculature, may select different media or compositions based on plaque composition (e.g., calcified versus non-calcified), may select different media or control settings when a stent is present, and/or may adjust the medium and/or treatment cycle timing based on an allowable occlusion time and corresponding ischemia risk.

In certain embodiments, the treatment system is configured to selectively control acoustic coupling and thermal delivery by coordinating (i) a volume and/or pressure state of an inner balloon and (ii) a volume and/or pressure state of an outer balloon. In such embodiments, the inner balloon and outer balloon together define a controllable multi-layer medium through which energy is transmitted to target tissue, and the controller is configured to adjust the respective inflation states to achieve desired acoustic, thermal, and procedural performance.

In embodiments configured to deliver intravascular lithotripsy energy, the outer balloon may contain an acoustic coupling medium or fluid (e.g., saline, water, contrast media, or mixtures thereof) and may contain or be coupled to one or more lithotripsy or shock wave emitters. The controller may adjust outer balloon pressure and/or volume to set a commanded balloon diameter and wall apposition while also controlling the thickness and/or distribution of fluid within the outer balloon. Additionally, the controller may adjust the inner balloon pressure and/or volume to change the geometry and/or residual volume within the overall balloon system (e.g., changing a thickness of a fluid layer between an emitter and a target region of tissue). By controlling one or more of: (i) the outer balloon fluid volume, (ii) the outer balloon pressure, (iii) the inner balloon inflation state, and/or (iv) the resulting thickness of fluid and/or gas layers within the balloon system, the system can control a coupling path length and acoustic attenuation between the emitter and the target tissue. For example, the controller may reduce a residual fluid volume or a coupling-layer thickness to reduce attenuation and increase acoustic coupling efficiency, or may increase such volume or thickness to reduce acoustic intensity or broaden the coupling region, depending on the procedure objective.

In certain intravascular procedures, prolonged balloon inflation may reduce or interrupt blood flow and may increase ischemic risk. In various embodiments, the controller is configured to manage a tradeoff between (i) achieving a commanded level of acoustic coupling and/or thermal dose and (ii) limiting an inflation duration and/or number of inflation cycles to reduce ischemic burden. For example, the controller may implement a treatment protocol comprising multiple cycles in which the balloons are inflated to a therapy state for a first interval, energy is delivered during all or a portion of that interval, and the balloons are then partially or fully deflated for a reperfusion interval. In some embodiments, the controller selects cycle timing, inflation pressure, inflation volume, and/or energy delivery timing based on one or more measured parameters such as pressure, temperature, delivered energy, estimated thermal dose, vessel diameter, lesion length, and/or patient-specific constraints. In some embodiments, the controller shortens a time-to-therapeutic effect by configuring the inner/outer balloon system to reduce coupling-path attenuation (for IVL) and/or to increase thermal contact area (for thermal therapy), thereby enabling a targeted therapy outcome with reduced inflation duration.

Accordingly, in various embodiments the inner balloon volume and the outer balloon pressure/volume are controlled as system variables that jointly determine (i) a lithotripsy coupling path and corresponding acoustic attenuation, (ii) a thermal contact-versus-insulation state and resulting heat flux, and (iii) a procedural cycle time that balances therapy effectiveness with ischemia risk. In this manner, the disclosed catheter system provides a coordinated, multi-variable control architecture for delivering acoustic and thermal therapies with improved efficiency, controllability, and safety.

In various embodiments, the outer balloon is configured to reduce ischemic burden during treatment by providing partial or intermittent perfusion rather than fully occluding the vessel for the entire treatment interval. In some embodiments, the catheter includes a perfusion pathway configured to permit blood flow past the inflated balloon segment while the outer balloon remains deployed against the vessel wall.

In some embodiments, the catheter includes a perfusion lumen extending through the catheter shaft and providing a proximal-to-distal shunt path, thereby allowing blood to bypass the treatment region during balloon inflation. In some embodiments, the outer balloon includes one or more side perfusion channels, grooves, or folds configured to maintain one or more flow paths along the balloon outer surface when the balloon is inflated. In some embodiments, the controller executes an intermittent perfusion protocol in which the outer balloon is periodically partially deflated and/or fully deflated to allow reperfusion between therapy intervals. In some embodiments, the outer balloon is configured for partial occlusion by providing controlled leakage through one or more microchannels, porous regions, or flow-restrictive features that permit a limited perfusion flow while maintaining balloon apposition and therapy delivery.

In various embodiments, perfusion-enabled operation is integrated with balloon cycling logic. For example, the controller may alternate between (i) an inflation state configured for thermal delivery and/or lithotripsy delivery and (ii) a reperfusion state configured to increase blood flow through or around the outer balloon, thereby reducing ischemia risk while achieving a prescribed thermal dose and/or lithotripsy pulse count. Such perfusion-enabled and cyclic operation may be particularly beneficial in coronary vasculature and other anatomies where allowable occlusion time is limited. In one embodiment, the balloons remain inflated for a period of time ranging from 30-120 seconds (or any time increment therein) and then are both deflated for a period of time ranging from 30-120 seconds (or any time increment therein) before being re-inflated again for a third period of time ranging from 30-120 seconds (or any time increment therein). This cycle would repeat in a range of 1 to 20 times. In an exemplary protocol, the balloons inflate for 30 seconds and then deflate for 30 seconds and then repeat this cycle as needed based on patient needs and clinician judgment.

In various embodiments, the inflation medium within the outer balloon is selected and/or controlled to tune both thermal and acoustic performance of the system. The outer balloon medium may be a gas, a liquid, or a mixture (including multi-phase mixtures), and the selected medium may be used to adjust (i) an effective thermal conductivity and thermal resistance between an inner balloon with an ablative liquid and blood and/or non-target tissue, and (ii) acoustic coupling and/or acoustic attenuation for embodiments configured to deliver intravascular lithotripsy (IVL) energy.

By way of example, in some embodiments a gas-filled outer balloon provides relatively higher thermal resistance, thereby increasing thermal insulation and reducing heat transfer to blood and/or non-target tissue, while also providing relatively poorer acoustic coupling for IVL energy transmission. In some embodiments a liquid-filled outer balloon provides relatively lower thermal resistance, thereby promoting more uniform heat transfer and/or enabling active cooling via circulation, while also providing improved acoustic coupling for IVL energy transmission. In some embodiments the outer balloon is filled with a mixture (e.g., a liquid with dissolved gas, a liquid/contrast mixture, a liquid with entrained microbubbles, or other mixtures) to provide intermediate and tunable thermal resistance and acoustic coupling characteristics.

In other embodiments, when lithotripsy pulses are delivered, the balloons are controlled to maintain a relatively thin coupling layer between the inner balloon and outer balloon, for example less than about 1 mm of fluid layer thickness, to reduce attenuation and improve lithotripsy coupling efficiency during pulse delivery.

With respect to certain therapeutic measures, after performing the presently disclosed therapies on a patient, 50-100% of the patient's intima in the lumen of the stent is removed or debulked and more than 10%, preferably more than 25%, of the patient's diseased intima, media, smooth muscle cells, and/or other tissue located beneath a stent is removed. After performing the presently disclosed therapies on a patient, more than 50% of the patient's tunica intima and/or more than 25% of the patient's tunica media in the target ablation zone is ablated without ablating more than 25% of the patient's tunica adventitia. After performing the presently disclosed therapies on a patient, certain biomarkers/pathways are modulated, including upregulating the expression of Bax (Bcl-2-associated X) protein by 50% relative to a pre-treatment state, down regulating the expression of Bcl-2 protein by 20% relative to a pre-treatment state, increasing the relative ratio of Bax to Bcl-2 by 20% relative to a pre-treatment state, upregulating the expression of Caspase-3 enzyme by 20% relative to a pre-treatment state, upregulating the expression of transforming growth factor beta-1 cytokine by 20% relative to a pre-treatment state, and upregulating the expression of SMAD2 signal-transducing transcription factor by 20% relative to a pre-treatment state. Additionally, after performing the presently disclosed therapies on a patient, the apoptosis rate (a quantitative measure of how many cells in a sample are undergoing programmed cell death, expressed as a percentage) is greater than 10% at 50° C., greater than 15% at 60° C., and greater than 20% at 70° C. while a control sample has an apoptosis rate of less than 5% at each of the aforementioned temperature levels, as determined by terminal deoxynucleotidyl transferase dUTP nick-end labeling assays that DNA strand breaks indicative of late-stage apoptosis.

The above examples are merely illustrative of the many applications of the system of present specification. Although only a few embodiments of the present invention have been described herein, it should be understood that the present invention might be embodied in many other specific forms without departing from the spirit or scope of the invention. Therefore, the present examples and embodiments are to be considered as illustrative and not restrictive, and the invention may be modified within the scope of the appended claims.

What is claimed is:

1. A system for treating in-stent restenosis in a lumen of a patient, comprising:
a catheter having a distal end, wherein the distal end comprises an inner inflatable member defining an inner chamber configured to receive a working liquid and an outer inflatable member enclosing the inner inflatable member and defining an outer chamber configured to contain an acoustic coupling fluid; and
a controller coupled to the catheter and configured to activate and control an acoustic wave source coupled to the distal end and configured to deliver lithotripsy pulses that propagate through the acoustic coupling fluid and through the outer inflatable member, activate and control a thermal energy source configured to heat the working liquid in the inner chamber;
and one or more pumps for delivering the working liquid and the acoustic coupling fluid to the distal end.

2. The system of claim 1, wherein the controller is further configured to control at least one of: (i) a parameter of the thermal energy source, (ii) a parameter of the acoustic wave source, or (iii) a pressure or a volume of the outer chamber.

3. The system of claim 2, wherein the controller is further configured to adjust the pressure or the volume of the outer chamber to control at least one of an amount of ablative energy passing from the inner chamber to the outer chamber or an acoustic coupling condition for the lithotripsy pulses comprising an acoustic path length or attenuation through the acoustic coupling fluid.

4. The system of claim 1, wherein the inner inflatable member is inflatable to a dilation pressure sufficient to dilate the lumen, and wherein the outer inflatable member is inflatable to a supra-dilation pressure greater than the dilation pressure.

5. The system of claim 1, wherein the acoustic wave source comprises at least one emitter disposed within the outer chamber or coupled to the distal end to transmit pulses into the acoustic coupling fluid.

6. The system of claim 1, wherein the controller is configured to perform a therapy sequence comprising causing the outer inflatable member to be inflated and the inner inflatable member to be inflated and causing the working liquid to be heated and delivered to the distal end during a first period, wherein, in the course of the first period, the working liquid's temperature is held substantially constant.

7. The system of claim 6, wherein the controller is configured to perform a therapy sequence comprising causing the acoustic wave source to deliver lithotripsy pulses that propagate through the acoustic coupling fluid during a second period.

8. The system of claim 7, wherein the second period initiates at a beginning of the first period and ends at an end of the first period.

9. The system of claim 7, wherein the second period initiates at a beginning of the first period and after 30% to 50% of the first period elapses.

10. The system of claim 7, wherein the second period initiates after 50% of the first period elapses and ends at an end of the first period.

11. A method for treating in-stent restenosis in a lumen of a patient, comprising:
providing a catheter having a distal end, wherein the distal end comprises an inner inflatable member defining an inner chamber configured to receive a working liquid and an outer inflatable member enclosing the inner inflatable member and defining an outer chamber configured to contain an acoustic coupling fluid;
using a controller, causing the acoustic coupling fluid to be delivered to the outer chamber;
using the controller, causing the working liquid to be delivered and heated in the inner chamber during a first period, wherein a temperature of the working liquid is substantially constant during the first period; and
using the controller, causing the acoustic wave source to deliver lithotripsy pulses that propagate through the acoustic coupling fluid during a second period.

12. The method of claim 11, wherein the second period initiates at a beginning of the first period and ends at an end of the first period.

13. The method of claim 11, wherein the second period initiates at a beginning of the first period and after 30% to 50% of the first period elapses.

14. The method of claim 11, wherein the second period initiates after 50% of the first period elapses and ends at an end of the first period.

15. The method of claim 11, wherein 50-100% of the patient's intima in the lumen is removed and more than 10% of the patient's intima, media, smooth muscle cells, and/or other tissue located beneath a stent is removed.

16. The method of claim 15, wherein more than 50% of the patient's tunica intima in the lumen and more than 25% of the patient's tunica media in the lumen is ablated without ablating more than 25% of the patient's tunica adventitia in the lumen.

17. The method of claim 11, further comprising controlling at least one of: (i) a parameter of the thermal energy source, (ii) a parameter of the acoustic wave source, or (iii) a pressure or a volume of the outer chamber.

18. The method of claim 17, further comprising adjusting the pressure or the volume of the outer chamber to control at least one of an amount of ablative energy passing from the inner chamber to the outer chamber or an acoustic coupling condition for the lithotripsy pulses comprising an acoustic path length or attenuation through the acoustic coupling fluid.

19. The method of claim 11, further comprising inflating the inner inflatable member to a dilation pressure sufficient to dilate the lumen, and inflating the outer inflatable member to a supra-dilation pressure greater than the dilation pressure.

20. The system of claim 11, wherein the acoustic wave source comprises at least one emitter disposed within the outer chamber or coupled to the distal end to transmit pulses into the acoustic coupling fluid.
